(19) United States
(12) Patent Application Publication
(10) Pub. No.: US 2024/0262924 A1
(43) Pub. Date: Aug. 8, 2024

(54) COMBINATION DOSAGE REGIME OF CD137 AND PD-L1 BINDING AGENTS

(71) Applicants: GENMAB A/S, Valby (DK); BioNTech SE, Mainz (DE)

(72) Inventors: Ugur SAHIN, Mainz (DE); Alexander MUIK, Mainz (DE); Ulf FORSSMANN, Valby (DK); Maria N. JURE-KUNKEL, Plainsboro, NJ (US); Gaurav BAJAJ, Plainsboro, NJ (US); Craig THALHAUSER, Bordentown, NJ (US); Nora PENCHEVA, Utrecht (NL)

(21) Appl. No.: 18/570,257

(22) PCT Filed: Jun. 20, 2022
(86) PCT No.: PCT/EP2022/066764

§ 371 Date: Dec. 14, 2023

Related U.S. Application Data
(60) Provisional application No. 63/257,879, filed on Oct. 20, 2021.
(60) Provisional application No. 63/212,781, filed on Jun. 21, 2021.

(51) Int. Cl. C07K 16/28; A61K 39/00; A61P 35/00

(52) U.S. Cl. CPC C07K 16/2878; A61P 35/00; C07K 16/2827; A61K 2039/505; A61K 2039/54; A61K 2039/545; C07K 2317/31

(57) ABSTRACT

The present invention relates to a method for reducing or preventing progression of a tumor or treating cancer in a subject, comprising administering to the subject a binding agent comprising a first antigen-binding region binding to human CD137, and a second antigen-binding region binding to human PD-L1. The invention further provides a binding agent for use in reducing or preventing progression of a tumor or for use in treatment of cancer.

### RELATED APPLICATIONS

This application is a 35 U.S.C. § 371 filing of International Patent Application No. PCT/EP2022/066764, filed Jun. 20, 2022, which claims priority to U.S. Provisional Patent Application Ser. Nos. 63/257,879, filed Oct. 20, 2021, and 63/212,781, filed Jun. 21, 2021, the entire disclosures of which are hereby incorporated herein by reference.

### SEQUENCE LISTING

The instant application contains a Sequence Listing which has been submitted electronically in ASCII format and is hereby incorporated by reference in its entirety. Said ASCII file, created on Dec. 13, 2023, is named 748497_GMB9-009US_ST25.txt and is 77,059 bytes in size.

### FIELD OF THE INVENTION

The present invention relates to a method for reducing or preventing progression of a tumor or treating cancer in a subject, comprising administering to said subject a binding agent comprising a first antigen-binding region binding to human CD137, and a second antigen-binding region binding to human PD-L1.

### BACKGROUND OF THE INVENTION

CD137 (4-1BB, TNFRSF9) is a member of the tumor necrosis factor (TNF) receptor (TNFR) family. CD137 is a co-stimulatory molecule on CD8⁺ and CD4⁺ T cells, regulatory T cells (Tregs), natural killer (NK) and NKT cells, B cells and neutrophils. On T cells, CD137 is not constitutively expressed, but induced upon T-cell receptor (TCR)-activation. Stimulation via its natural ligand 4-1BBL or agonist antibodies leads to signaling using TNFR-associated factor (TRAF)-2 and TRAF-1 as adaptors. Early signaling by CD137 involves K-63 poly-ubiquitination reactions that ultimately result in activation of the nuclear factor (NF)-κB and mitogen-activated protein (MAP)-kinase pathways. Signaling leads to increased T cell co-stimulation, proliferation, cytokine production, maturation and prolonged CD8⁺ T-cell survival. Agonistic antibodies against CD137 have been shown to promote anti-tumor control by T cells in various pre-clinical models (Murillo et al. 2008 Clin. Cancer Res. 14(21): 6895-6906). Antibodies stimulating CD137 can induce survival and proliferation of T cells, thereby enhancing the anti-tumor immune response. Antibodies stimulating CD137 have been disclosed in the prior art, and include urelumab, a human IgG4 antibody (WO2005035584) and utomilumab, a human IgG2 antibody (Fisher et al. 2012 Cancer Immunol. Immunother. 61: 1721-1733).

Programmed death ligand 1 (PD-L1, PDL1, CD274, B7H1) is a 33 kDa, single-pass type I membrane protein. Three isoforms of PD-L1 have been described, based on alternative splicing. PD-L1 belongs to the immunoglobulin (Ig) superfamily and contains one Ig-like C2-type domain and one Ig-like V-type domain. Freshly isolated T and B cells express negligible amounts of PD-L1 and a fraction (about 16%) of CD14⁺ monocytes constitutively express PD-L1. However, interferon-γ (IFNγ) is known to upregulate PD-L1 on tumor cells.

PD-L1 obstructs anti-tumor immunity by 1) tolerizing tumor-reactive T cells by binding to its receptor, programmed cell death protein 1 (PD-1) (CD279) on activated T cells; 2) rendering tumor cells resistant to CD8⁺ T cell and Fas ligand-mediated lysis by PD-1 signaling through tumor cell-expressed PD-L1; 3) tolerizing T cells by reverse signaling through T cell-expressed CD80 (B7.1); and 4) promoting the development and maintenance of induced T regulatory cells. PD-L1 is expressed in many human cancers, including melanoma, ovarian, lung and colon cancer (Latchman et al., 2004 Proc Natl Acad Sci USA 101, 10691-6).

WO2019/025545 provides multispecific antibodies that can bind both PD-L1 and CD137. These antibodies are designed to simultaneous bind to PD-L1-expressing antigen-presenting cells (APCs) or tumor cells and CD137-expressing T cells. By combining checkpoint blockade with 4-1BB-dependent T-cell activation, the multispecific antibodies enhance proliferation and cytokine production of activated T-cells, activate immune cells in the tumor-draining lymph nodes, and induce tumor regression in vivo.

However, despite the advanced mode of action and proven efficacy in vivo of such multispecific antibodies, it is still desirable to provide means for further improving their efficacy in vivo.

### SUMMARY OF THE INVENTION

A clinical trial (ClinicalTrials.gov Identifier: NCT03917381) including dose escalation and expansion was designed to determine e.g. the recommended phase 2 dose (RP2D) as well as the safety, tolerability, pharmacokinetics (PK), and anti-tumor activity of a multispecific antibody that binds PD-L1 and CD137. The present inventors also developed a Pharmacokinetic/Pharmacodynamic (PK/PD) model predicting trimer formation (crosslinking of the multispecific binding agent to PD-L1 and 4-1BB) and receptor occupancy (RO) for PD-L1 and 4-1BB in the tumor.

Using the PK/PD model, the inventors were able to show that trimer formation in the tumor peaks at a dose of 100 mg when the multispecific antibody is administered e.g. every third week. Dosing the multispecific antibody at 500 mg on a less frequent basis; e.g. every 6 weeks, is predicted to provide higher PD-L1 receptor occupancy with intermittent 4-1BB activation as trimers are engaged to a lesser extent compared with the 100 mg dose.

In the clinical trial, the multispecific antibody demonstrated a manageable safety profile and preliminary clinical activity in a population with advanced solid tumors. Clinical pharmacodynamic data showed higher magnitude and consistent modulation of peripheral pharmacodynamic endpoints at dose levels ≤200 mg and clinical data from expansion showed that the dose of 100 mg administered every third week resulted in responses within first 2 cycles. A dosing scheme which combines frequent dosing of the multispecific antibody at 100 mg with less frequent, higher maintenance doses, e.g. of 500 mg is expected to provide improved duration of response. In addition, the higher doses of the multispecific antibody are predicted to engage less trimers in liver compared to 100 mg Q3W and therefore expected to have a better safety profile.

Hence, in the first aspect, the invention provides for a method for reducing or preventing progression of a tumor or treating cancer in a subject, comprising administering to said subject a binding agent comprising a first antigen-binding region binding to human CD137, such as human CD137 consisting of the amino acid sequence set forth in SEQ ID NO: 24, and a second antigen-binding region binding to human PD-L1, such as human PD-L1 consisting of the amino acid sequence set forth in SEQ ID NO: 26, wherein
said binding agent is administered to the subject in a dosing schedule that comprises administration of Dose A in one or more treatment cycles and administration of dose B in one or more treatment cycles,
the amount of binding agent in Dose A being
a) about 0.3-2.5 mg/kg body weight, or about 25-200 mg in total; and/or
b) about 2.1×10−9-1.7×10−8 mol/kg body weight, or about 1.7×10−7-1.48×10−6 mol in total; and
the amount of binding agent in Dose B being
c) about 3.8-7.5 mg/kg body weight, or about 300-600 mg in total; and/or
d) about 2.6×10−8-5.1×10−8 mol/kg body weight, or about 2.0-4.1×10−6 mol in total.
   The amount of said binding agent in Dose A is preferably
a) about 1.25 mg/kg body weight, or about 100 mg in total; and/or
b) about 8.5×10⁻⁹ mol/kg body weight, or about 6.8×10⁻⁷ mol in total.
   The amount of binding agent in Dose B is preferably
a) about 6.25 mg/kg body weight, or about 500 mg in total; and/or
b) about 4.3×10⁻⁸ mol/kg body weight, or about 3.4×10⁻⁶ mol in total.

In a further aspect, the invention relates to a binding agent for use in reducing or preventing progression of a tumor or for use in treatment of cancer, wherein the binding agent comprises a first antigen-binding region binding to human CD137, such as human CD137 consisting of the amino acid sequence set forth in SEQ ID NO: 24, and a second antigen-binding region binding to human PD-L1, such as human PD-L1 consisting of the amino acid sequence set forth in SEQ ID NO: 26, and the binding agent is administered to the subject in a dosing schedule that comprises administration of Dose A in one or more treatment cycles and administration of dose B in one or more treatment cycles,
the amount of binding agent in Dose A being
a) about 0.3-2.5 mg/kg body weight, or about 25-200 mg in total; and/or
b) about 2.1×10−9-1.7×10−8 mol/kg body weight, or about 1.7×10−7-1.4×10−6 mol in total; and
the amount of binding agent in Dose B being
c) about 3.8-7.5 mg/kg body weight, or about 300-600 mg in total; and/or
d) about 2.6×10−8-5.1×10−8 mol/kg body weight, or about 2.0-4.1×10−6 mol in total.

These and other aspects and embodiments of the invention, including are described in further detail below.

### BRIEF DESCRIPTION OF THE DRAWINGS

FIG. 1: Simultaneous binding of GEN1046 to PD-L1- and CD137-expressing K562 cells induces doublet formation with a bell-shaped dose-response curve. Equal numbers of CellTrace™ Far Red labelled K562 cells transgenic for CD137 (K562_h4-1BB) were co-incubated with CellTrace™ Violet labelled K562 cells transgenic for PD-L1 (K562_hPD-L1) in the presence of 0.001-100 μg/mL i) GEN1046 or ii) a combination of control antibodies PD-L1-547-FEALxb12-FEAR and b12-FEALxCD137-009-HC7LC2-FEAR for 15 minutes. Samples were analyzed by flow cytometry and the percent CellTrace™ Far Red/CellTrace™ Violet double-positive doublets (A) plotted as a function of GEN1046 concentration (B). Data shown are mean +standard deviation of n=3 technical replicates (perhaps the symbol needs to be smaller to show the SD).

FIG. 2: Schematic representation of the anticipated mode of action of CD137xPD-L1 bispecific antibodies. (A) PD-L1 is expressed on antigen-presenting cells (APCs) as well as on tumor cells. PD-L1 binding to T cells expressing the negative regulatory molecule PD-1 effectively overrides T cell activation signals and eventually leads to T cell inhibition. (B) Upon addition of a CD137xPD-L1 bispecific antibody, the inhibitory PD-1: PD-L1 interaction is blocked via the PD-L1-specific arm and at the same time, the bispecific antibody, through the cell-cell interaction provides agonistic signaling to CD137 expressed on the T cells resulting in strong T cell costimulation.

FIG. 3: Relative luminescence units (RLU) as a function of antibody concentration in a luciferase-based CD137-activation reporter assay performed in the presence of PD-L1 expressing tumor cell lines. Endogenously PD-L1 expressing human ovarian cancer cell line ES-2 (A) and breast cancer cell line MDA-MB-231 (B) were co-cultivated with NFkB-Luc2P/4-1BB Jurkat reporter cells in the presence of 0.00128-100 μg/mL i) GEN1046 or ii) b12-FEAL control antibody for 6 hours. Luciferase expression induction was determined by incubation with a luciferase substrate and measurement of relative luminescence units. Data shown are mean±standard deviation of n=3 technical replicates.

FIG. 4: Comparison of GEN1046 with control antibodies PD-L1-547-FEALxb12-FEAL or IgG1-b12-FEAL in a polyclonal T-cell proliferation assay. CFSE-labeled PBMCs were incubated with sub-optimal concentration of anti-CD3 antibody (0.03 μg/mL), and cultured in the presence of 0.0032-10 μg/mL i) GEN1046 ii) PD-L1-547-FEALxb12-FEAR or iii) b12-FEAL control antibody for four days. T-cell proliferation of total T cells (A) and CCR7+CD45RO+ central memory and CCR7−CD45RO+ effector memory T-cell subsets in total T cells (B) was measured by flow cytometry. Data are shown from one representative donor as the mean expansion index of two replicates, as calculated using FlowJo v10.4 software. Error bars (SD) indicate the variation within the experiment (two replicates, using cells from one donor).

FIG. 5: Release of the PD-1/PD-L1-mediated T-cell inhibition and additional co-stimulation of CD8⁺ T-cell proliferation by GEN1046 in an antigen-specific T-cell assay with active PD-1/PD-L1 axis. CD8⁺ T cells were electroporated with RNA encoding the alpha and beta chains of the CLDN6-specific TCR (10 μg each) either with RNA encoding PD-1 (0.4-10 μg) or without (w/o PD-1), labeled with CFSE and co-cultured with immature DC that were electroporated with 0.3 μg (A) or 1 μg (B) RNA encoding CLDN6. Electroporated CD8⁺ T cells and iDC were co-cultured in the presence of GEN1046 (0.00015-1 μg/mL) or b12-FEAL (1 μg/mL) for 4 days. T-cell proliferation was assessed by analyzing CFSE dilution in CD8⁺ T cells using flow cytometry and the T-cell expansion index (e.g. how much the total T cell population has expanded by proliferation) was automatically calculated by FlowJo (version 10.3). Data shown are mean expansion index±SD of triplicate wells from one donor out of four donors included in two experiments.

FIG. 6: Effect of GEN1046 on secretion of pro-inflammatory cytokines (IFNγ, TNFα, IL-13 and IL-8) in an antigen-specific T-cell assay with or without PD-1 electroporation into T cells. CD8⁺ T cells were electroporated with RNA encoding the alpha and beta chains of the CLDN6-specific TCR (10 μg each) either with RNA encoding PD-1 (2 μg) or without (w/o PD-1), labeled with CFSE and co-cultured with immature DC that were electroporated with 1 μg RNA encoding CLDN6. Electroporated CD8⁺ T cells and iDC were co-cultured in the presence of GEN1046 (0.00015-1 μg/mL) or b12-FEAL (1 μg/mL). Cytokine levels of supernatants were determined 48 hours after antibody addition by multiplex sandwich immunoassay using the MSD V-Plex Human Proinflammatory panel 1 (10-Plex) kit. Data shown are mean concentration±SD of sextuplicate wells from one representative donor of two donors included in the experiment.

FIG. 7: Ex vivo expansion of tumor infiltrating lymphocytes (TIL) from a human non-small-cell lung cancer tissue resection by CD137-009-FEALxPD-L1-547-FEAR. Tumor pieces from the resected tissue were cultured with 10 U/ml IL-2 and the indicated concentration of CD137-009-FEALxPD-L1-547-FEAR. After 10 days of culture, cells were harvested and analyzed by flow cytometry. (A) TIL count per 1,000 beads, (B) CD3+CD8+ T cell count per 1,000 beads, (C) CD3+CD4+ T cell count per 1,000 beads, (D) CD3-CD56+ NK cell count per 1,000 beads. Data shown are mean cell counts±SD of five individual wells, with two tumor pieces per well as starting material. *p<0.05 using ordinary one-way ANOVA with Dunnett's multiple comparisons test.

FIG. 8: Pharmacodynamic assessments, including changes in circulating levels of interferon-gamma (IFN-γ) and interferon-gamma-inducible protein 10 IP-10 (A-B), proliferating effector memory CD8 T cells and total CD8 T cells (C-D), were conducted using blood samples from patients with advanced solid tumors enrolled in the dose escalation phase of an open-label, multi-center safety trial of GEN1046 (NCT03917381; Data cut off: Jan. 19 2021).
A-B. Circulating levels of IFN-γ and IP-10 were measured in serum samples at baseline, and at multiple timepoints post administration of GEN1046 in cycle 1 and cycle 2 (days 1 [2 h and between 4-6 h post-administration], 2, 3, 8, and 15). IFN-γ and IP-10 levels in serum samples were determined by Meso Scale Discovery (MSD) multiplex immune assay. Data shown are the maximal fold-change from baseline measured during cycle 1. Statistical analysis was performed using the Wilcoxon-Mann-Whitney test.
C-D. Immunophenotyping of peripheral blood was conducted in whole blood collected at baseline and at multiple timepoints post administration of GEN1046 in cycle 1 and cycle 2 (days 2, 3, 8 and 15). The frequency of proliferating (Ki67⁺) total CD8 T cells and effector memory CD8 T cells (CD8⁺CD45RA⁻CCR7⁻ T cells) were assessed in whole blood samples by flow cytometry. Data shown are the maximal fold-change from baseline measured during cycle 1. Statistical analysis was performed using the Wilcoxon-Mann-Whitney test.

FIG. 9: Schematic outline of clinical trial design.

FIG. 10: Dose escalation; best percent change from baseline in tumor size, all patients. Data cut-off: Sep. 29, 2020. Post-baseline scans were not conducted for five patients. ^{a}Minimum duration of response (5 weeks) per RECIST v1.1 not reached. ^{b}PR was not confirmed on a subsequent scan. NE, non-evaluable; NSCLC, non-small cell lung cancer; PD, progressive disease; PD-(L)1, programmed death (ligand) 1; PR, partial response; SD, stable disease; SoD, sum of diameters; uPR, unconfirmed partial response.

FIG. 11: Dose escalation; Best change from baseline in tumor size, patients with NSCLC. Data cut-off: Sep 29, 2020.
^{a}PR was not confirmed by a subsequent scan.
bPD-L1 expression was assessed in archival tumor specimens.
BOR, best overall response; CR, complete response; ICI, immune checkpoint inhibitor; NA, not available; PD, progressive disease; PD-(L)1, programmed death (ligand) 1; PR, partial response; RECIST, Response Evaluation Criteria in Solid Tumors; SD, stable disease; SoD, sum of diameters; TPS, tumor proportion score; uPR, unconfirmed partial response.

FIG. 12: Model Predicted Maximal Trimer Formation and Receptor Occupancy for PD-L1 at 100 mg dose administered once every third week (Q3W) and at 500 mg dose administered once every 6 weeks (Q6W).

FIG. 13: Expansion cohort 1, Data cut-off: Jan. 29, 2021:
A) Best change from baseline in tumor size: NE, non-evaluable; PD, progressive disease; SD, stable disease; uPR, unconfirmed partial response, ⋄=Prior PD-(L)1; PD-(L)1, programmed death (ligand) 1; RECIST, Response Evaluation Criteria in Solid Tumors ;
B) Target lesion Sum of Diameters (SoD) change from baseline. Black lines represent subject mounting progressive disease. Gray lines represent subjects mounting a best overall response of stable disease (SD) or, where indicated, partial response (PR). Black triangles, circles and squares indicate time point responses of progressive disease, stable disease and partial response, respectively. Open circles represent “not evaluable”; i.e. patient has progressed but is treated and followed up by scans after RECIST based progression; NL indicates “new lesion”.

FIG. 14: Expansion cohort 2, Data cut-off: Jan. 29, 2021:
A) Best change from baseline in tumor size: PD, progressive disease; SD, stable disease; RECIST, Response Evaluation Criteria in Solid Tumors; SD, stable disease; uPR, unconfirmed partial response,; RECIST, Response Evaluation Criteria in Solid Tumors
B) Target lesion Sum of Diameters (SoD) change from baseline. Black lines represent subject mounting progressive disease. Gray lines represent subjects mounting a best overall response of stable disease. Black triangles and circles indicate time point responses of progressive disease and stable disease, respectively.

FIG. 15: Expansion cohort 3, Data cut-off: Jan. 29, 2021:
A) Best change from baseline in tumor size: NE, non-evaluable; PD, progressive disease; SD, stable disease; ⋄=Prior PD-(L)1; PD-(L)1, programmed death (ligand) 1; RECIST, Response Evaluation Criteria in Solid Tumors;
B) Target lesion Sum of Diameters (SoD) change from baseline. Black lines represent subject mounting progressive disease. Gray lines represent subjects mounting a best overall response of stable disease. Black triangles and circles indicate time point responses of progressive disease and stable disease, respectively.

FIG. 16: Expansion cohort 4, Data cut-off: Jan. 29, 2021:
A) Best change from baseline in tumor size: PD, progressive disease; SD, stable disease; PR, partial response; RECIST, Response Evaluation Criteria in Solid Tumors;
B) Target lesion Sum of Diameters (SoD) change from baseline. Black lines represent subject mounting progressive disease. Gray lines represent subjects mounting a best overall response of stable disease or, where indicated, partial response (PR). Black triangles, circles and squares indicate time point responses of progressive disease, stable disease and partial response, respectively. Open circles represent “not evaluable”; i.e. patient has progressed but is treated and followed up by scans after RECIST based progression.

FIG. 17: Expansion cohort 5, Data cut-off: Jan. 29, 2021:
A) Best change from baseline in tumor size: NE, non-evaluable; PD, progressive disease; SD, stable disease; uPR, unconfirmed partial response, ⋄=Prior PD-(L)1; PD-(L)1, programmed death (ligand) 1; RECIST, Response Evaluation Criteria in Solid Tumors;
B) Target lesion Sum of Diameters (SoD) change from baseline. Black lines represent subject mounting progressive disease. Gray lines represent subjects mounting a best overall response of stable disease (SD) or, where indicated, partial response (PR). Black triangles, circles and squares indicate time point responses of progressive disease, stable disease and partial response, respectively.

FIG. 18: Expansion cohort 6, Data cut-off: Jan. 29, 2021:
A) Best change from baseline in tumor size; NE, non-evaluable; PD, progressive disease; SD, stable disease; uPR, unconfirmed partial response, ⋄=Prior PD-(L)1; PD-(L)1, programmed death (ligand) 1; RECIST, Response Evaluation Criteria in Solid Tumors;
B) Target lesion Sum of Diameters (SoD) change from baseline. Black lines represent subject mounting progressive disease. Gray lines represent subjects mounting a best overall response of stable disease (SD) or, where indicated, partial response (PR). Black triangles, circles and squares indicate time point responses of progressive disease, stable disease and partial response, respectively. Open circles represent “not evaluable”; i.e. patient has progressed but is treated and followed up by scans after RECIST based progression.

FIG. 19: Expansion cohort 7, Data cut-off: Jan. 29, 2021:
A) Best change from baseline in tumor size: PD, progressive disease, PR, partial response; SD, stable disease; RECIST, Response Evaluation Criteria in Solid Tumors;
B) Target lesion Sum of Diameters (SoD) change from baseline. Black lines represent subject mounting progressive disease. Gray lines represent subjects mounting a best overall response of stable disease or, where indicated, partial response (PR); Black triangles, circles and squares indicate time point responses of progressive disease, stable disease and partial response, respectively. Open circles represent “not evaluable”; i.e. patient has progressed but is treated and followed up by scans after RECIST based progression.

FIG. 20: Waterfall plot showing progression-free survival in subjects having received prior therapy with a checkpoint inhibitor (gray line) and checkpoint inhibitor naiive patients (black line).

FIG. 21: Comparison of time since last prior anti-PD-(L)1 in subjects across CPI-experienced expansion cohorts (GEN1046 monotherapy) with clinical response (PR), compared to those with stable disease (SD) or progressive disease (PD). Response groups were compared using a Wilcoxon test. PR vs. PD: p=0.0017; PR vs. SD: p=0.034.

### DETAILED DESCRIPTION OF THE INVENTION

### Definitions

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (abbreviated herein as C_{H} or CH). The heavy chain constant region typically is comprised of three domains, CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as V_{L} or VL) and a light chain constant region (abbreviated herein as C_{L} or CL). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, CDR sequences herein are identified according to IMGT rules using DomainGapAlign (Lefranc M P., Nucleic Acids Research 1999;27:209-212 and Ehrenmann F., Kaas Q. and Lefranc M.-P. Nucleic Acids Res., 38, D301-307 (2010); see also internet http address www.imgt.org/). Unless otherwise stated or contradicted by context, reference to amino acid positions in the constant regions in the present invention is according to the EU-numbering (Edelman et al., Proc Natl Acad Sci U S A. 1969 May; 63(1): 78-85; Kabat et al., Sequences of Proteins of Immunological Interest, Fifth Edition. 1991 NIH Publication No. 91-3242).

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present invention.

The term “binding agent” in the context of the present invention refers to any agent capable of binding to desired antigens. In certain embodiments of the invention, the binding agent is an antibody, antibody fragment, or construct thereof. The binding agent may also comprise synthetic, modified or non-naturally occurring moieties, in particular non-peptide moieties. Such moieties may, for example, link desired antigen-binding functionalities or regions such as antibodies or antibody fragments. In one embodiment, the binding agent is a synthetic construct comprising antigen-binding CDRs or variable regions.

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The term “antigen-binding region”, wherein used herein, refers to the region which interacts with the antigen and comprises both a VH region and a VL region. The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody herein, unless otherwise stated or clearly contradicted by context, includes fragments of an antibody that are antigen-binding fragments, i.e., retain the ability to specifically bind to the antigen. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al; Trends Biotechnol. 2003 Nov;21(11):484-90); (vi) camelid or Nanobody molecules (Revets et al; Expert Opin Biol Ther. 2005 Jan;5(1) : 111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques. An antibody as generated can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody of the invention may be a sequence variant of a naturally-occurring IgG1 antibody, including variations in the constant regions.

The term “monoclonal antibody” as used herein refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

The term “bispecific antibody” or “bs” in the context of the present invention refers to an antibody having two different antigen-binding regions defined by different antibody sequences. In some embodiments, said different antigen-binding regions bind different epitopes on the same antigen. However, in preferred embodiments, said different antigen-binding regions bind different target antigens. A bispecific antibody can be of any format, including any of the bispecific antibody formats described herein below.

When used herein, unless contradicted by context, the term “Fab-arm” or “arm” includes one heavy chain-light chain pair and is used interchangeably with “half-molecule” herein.

When a bispecific antibody is described to comprise a half-molecule antibody “derived from” a first antibody, and a half-molecule antibody “derived from” a second antibody, the term “derived from” indicates that the bispecific antibody was generated by recombining, by any known method, said half-molecules from each of said first and second antibodies into the resulting bispecific antibody. In this context, “recombining” is not intended to be limited by any particular method of recombining and thus includes all of the methods for producing bispecific antibodies described herein below, including for example recombining by half-molecule exchange, as well as recombining at nucleic acid level and/or through co-expression of two half-molecules in the same cells.

The term “monovalent antibody” means in the context of the present invention that an antibody molecule is capable of binding a single molecule of an antigen, and thus is not capable of crosslinking antigens or cells.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

When used herein, unless contradicted by context, the term “Fc region” refers to an antibody region consisting of the two Fc sequences of the heavy chains of an immunoglobulin, wherein said Fc sequences comprise at least a hinge region, a CH2 domain, and a CH3 domain.

When used herein, the term “heterodimeric interaction between the first and second CH3 regions” refers to the interaction between the first CH3 region and the second CH3 region in a first-CH3/second-CH3 heterodimeric protein.

When used herein, the term “homodimeric interactions of the first and second CH3 regions” refers to the interaction between a first CH3 region and another first CH3 region in a first-CH3/first-CH3 homodimeric protein and the interaction between a second CH3 region and another second CH3 region in a second-CH3/second-CH3 homodimeric protein.

As used herein, the terms “binding” or “capable of binding” in the context of the binding of an antibody to a predetermined antigen or epitope typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when determined using Bio-Layer Interferometry (BLI) or, for instance, when determined using surface plasmon resonance (SPR) technology in a BIAcore 3000 instrument using the antigen as the ligand and the antibody as the analyte. The antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein) other than the predetermined antigen or a closely-related antigen. The amount with which the affinity is higher is dependent on the K_{D} of the antibody, so that when the K_{D} of the antibody is very low (that is, the antibody is highly specific), then the degree to which the affinity for the antigen is lower than the affinity for a non-specific antigen may be at least 10,000-fold.

The term “k_{d}” (sec-1), as used herein, refers to the dissociation rate constant of a particular antibody-antigen interaction. Said value is also referred to as the k_{off} value.

The term “K_{D}” (M), as used herein, refers to the dissociation equilibrium constant of a particular antibody-antigen interaction.

The antibody used according to the invention may be an isolated antibody. An “isolated antibody” as used herein, is intended to refer to an antibody which is substantially free of other antibodies having different antigenic specificities. In a preferred embodiment, an isolated bispecific antibody that specifically binds to PD-L1 and CD137 is substantially free of monospecific antibodies that specifically bind to PD-L1 or CD137. In another preferred embodiment, the antibody, or a pharmaceutical composition comprising the antibody, is substantially free of naturally-arising antibodies that are not capable of binding to PD-L1. In a further preferred embodiment, the antibody of the invention possesses a structural change in its amino acid sequence, relative to the structure of a naturally-occurring anti-PD-L1 antibody, wherein said structural change causes said antibody to exhibit an altered functionality relative to the functionality exhibited by said naturally-occurring anti-PD-L1 antibody, said functionality being selected from the group consisting of: (i) PD-L1 binding affinity, (ii) ability to inhibit binding of PD-L1 to PD-1, (iii) ability to induce Fc-mediated effector functions and (iv) ability to not induce Fc-mediated effector functions.

The term “PD-L1” when used herein, refers to the Programmed Death-Ligand 1 protein. PD-L1 is found in humans and other species, and thus, the term “PD-L1” is not limited to human PD-L1 unless contradicted by context. Human, macaque (cynomolgus monkey), African elephant, wild boar and mouse PD-L1 sequences can be found through Genbank accession no. NP_054862.1, XP_005581836, XP_003413533, XP_005665023 and NP_068693, respectively. The sequence of human PD-L1 is also shown in SEQ ID NO: 25, wherein amino acids 1-18 are predicted to be a signal peptide. The mature sequence of human PD-L1 is provided in SEQ ID NO: 26.

The term “PD-1” when used herein, refers to the human Programmed Death-1 protein, also known as CD279.

The term “CD137” as used herein, refers to the human Cluster of Differentiation 137 protein. CD137 (4-1BB), also referred to as TNFRSF9, is the receptor for the ligand TNFSF9/4-1BBL. CD137 is believed to be involved in T cell activation. In one embodiment, CD137 is human CD137, having UniProt accession number Q07011. The sequence of human CD137 is also shown in SEQ ID NO: 23, wherein amino acids 1-23 are predicted to be a signal peptide. The mature sequence of human CD137 is provided in SEQ ID NO: 24.

The percent identity between two sequences is a function of the number of identical positions shared by the sequences (i.e., % homology = # of identical positions/total # of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

In the context of the present invention, conservative substitutions may be defined by substitutions within the classes of amino acids reflected in the following table:

TABLE 1
Amino acid residue classes for conservative substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), and |
| | Ile (I) |
| Non-polar Uncharged Residues | Cys (C), Met (M), and Pro (P) |
| Aromatic Residues | Phe (F), Tyr (Y), and Trp (W) |

In the context of the present invention, the following notations are, unless otherwise indicated, used to describe a mutation: i) substitution of an amino acid in a given position is written as e.g. K409R which means a substitution of a lysine in position 409 of the protein with an arginine; and ii) for specific variants the specific three or one letter codes are used, including the codes Xaa and X to indicate any amino acid residue. Thus, the substitution of lysine with arginine in position 409 is designated as: K409R, and the substitution of lysine with any amino acid residue in position 409 is designated as K409X. In case of deletion of lysine in position 409 it is indicated by K409 *.

In the context of the present invention, “inhibition of PD-L1 binding to PD-1” refers to any detectably significant reduction in the binding of PD-L1 to PD-1 in the presence of an antibody capable of binding PD-L1. Typically, inhibition means an at least about 10% reduction, such as an at least about 15%, e.g. an at least about 20%, such as an at least 40% reduction in binding between PD-L1 and PD-1, caused by the presence of an anti-PD-L1 antibody. Inhibition of PD-L1 binding to PD-1 may be determined by any suitable technique. In one embodiment, inhibition is determined as described in Example 6 in WO 2019/025545.

The term “specificity” as used herein is intended to have the following meaning unless contradicted by context. Two antibodies have the “same specificity” if they bind to the same antigen and the same epitope.

The term “epitope” means a protein determinant capable of specific binding to an antibody. Epitopes usually consist of surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked or covered by the specifically antigen binding peptide (in other words, the amino acid residue is within the footprint of the specifically antigen binding peptide).

The term “chimeric antibody” as used herein, refers to an antibody wherein the variable region is derived from a non-human species (e.g. derived from rodents) and the constant region is derived from a different species, such as human. Chimeric monoclonal antibodies for therapeutic applications are developed to reduce antibody immunogenicity. The terms “variable region” or “variable domain” as used in the context of chimeric antibodies, refer to a region which comprises the CDRs and framework regions of both the heavy and light chains of the immunoglobulin. Chimeric antibodies may be generated by using standard DNA techniques as described in Sambrook et al., 1989, Molecular Cloning: A laboratory Manual, New York: Cold Spring Harbor Laboratory Press, Ch. 15. The chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “human antibody” as used herein, refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse or rat, have been grafted onto human framework sequences. Human monoclonal antibodies can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known. Human monoclonal antibodies can thus e.g. be generated using transgenic or transchromosomal mice or rats carrying parts of the human immune system rather than the mouse or rat system. Accordingly, in one embodiment, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogenous animal antibody machinery, see e.g. Mendez et al. 1997 Nat Genet. 15(2): 146-56. The term “reducing conditions” or “reducing environment” refers to a condition or an environment in which a substrate, here a cysteine residue in the hinge region of an antibody, is more likely to become reduced than oxidized.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease.

The term “first-line treatment” refers to the initial, or first treatment recommended for a disease or illness. This may also be referred to as first-line therapy, primary treatment, initial treatment, or induction therapy.

The term “second-line treatment” refers to treatment for a disease or condition after the initial treatment of the subject (first-line treatment) has failed, the subject has relapsed or the disease has progressed, or the subject has experienced unacceptable adverse or side effects.

A “subject” includes any human or non-human animal. The term “non-human animal” includes, but is not limited to, vertebrates such as non-human primates, sheep, dogs, and rodents such as mice, rats, and guinea pigs. The terms “subject” and “patient” and “individual” are used interchangeably herein.

The response to treatment with the binding agent of the invention may be determined according to the Response Evaluation Criteria In Solid Tumors; version 1.1 (RECIST Criteria v1.1). The RECIST Criteria are set forth in table 2 below.

TABLE 2
Definition of Response (RECIST Criteria v1.1)

| | Category | Criteria |
|---|---|---|
| Based | Complete | Disappearance of all target lesions. Any |
| on | Response | pathological lymph nodes must have reduction |
| target | (CR) | in short axis to <10 mm. |
| lesions | Partial | ≥30% decrease in the sum of the LD of target |
| | Response | lesions, taking as reference the baseline sum |
| | (PR) | LD. |
| | Stable | Neither sufficient shrinkage to qualify for PR |
| | Disease | nor sufficient increase to qualify for PD, taking |
| | (SD) | as reference the smallest sum of LDs since the |
| | | treatment started. |
| | Progressive | ≥20% increase in the sum of the LDs of target |
| | Disease (PD) | lesions, taking as reference the smallest sum of |
| | | the LDs recorded since the treatment started or |
| | | the appearance of one or more new lesions. |
| Based | CR | Disappearance of all non-target lesions and |
| on non- | | normalization of tumor marker level. All lymph |
| target | | nodes must be non-pathological in size (<10 |
| lesions | | mm short axis). |
| | SD | Persistence of one or more non-target lesion(s) |
| | | or/and maintenance of tumor marker level above |
| | | the normal limits. |
| | PD | Appearance of one or more new lesions and/or |
| | | unequivocal progression of existing non-target |
| | | lesions. |

The “best overall response” is the best response recorded from the start of the treatment until disease progression/recurrence (the smallest measurements recorded since the treatment started will be used as the reference for PD). Subjects with CR or PR are considered to be objective response. Subjects with CR, PR or SD are considered to be in disease control. Subjects with NE are counted as non-responders. The best overall response is the best response recorded from the start of the treatment until disease progression/recurrence (the smallest measurements recorded since the treatment started will be used as the reference for PD).

“Duration of response (DOR)” only applies to subjects whose confirmed best overall response is CR or PR and is defined as the time from the first documentation of objective tumor response (CR or PR) to the date of first PD or death due to underlying cancer.

“Progression-free survival (PFS)” is defined as the number of days from Day 1 in Cycle 1 to the first documented progression or death due to any cause.

“Overall survival (OS)” is defined as the number of days from Day 1 in Cycle 1 to death due to any cause. If a subject is not known to have died, then OS will be censored at the latest date the subject was known to be alive (on or before the cut-off date).

The term “pharmaceutically acceptable” indicates that the substance or composition must be compatible chemically and/or toxicologically, with the other ingredients comprising a formulation, and/or the mammal being treated therewith.

As described above, in a first aspect, the invention relates to a method for reducing or preventing progression of a tumor or treating cancer in a subject, comprising administering to said subject a binding agent comprising a first antigen-binding region binding to human CD137, such as human CD137 consisting of the amino acid sequence set forth in SEQ ID NO: 24, and a second antigen-binding region binding to human PD-L1, such as human PD-L1 consisting of the amino acid sequence set forth in SEQ ID NO: 26, wherein
said binding agent is administered to the subject in a dosing schedule that comprises administration of Dose A in one or more treatment cycles and administration of dose B in one or more treatment cycles,
the amount of binding agent in Dose A being
   a) about 0.3-2.5 mg/kg body weight, or about 25-200 mg in total; and/or
   b) about 2.1×10⁻⁹-1.7×10⁻⁸ mol/kg body weight, or about 1.7×10⁻⁷-1.4×10⁻⁶ mol in total; and
the amount of binding agent in Dose B being
   c) about 3.8-7.5 mg/kg body weight, or about 300-600 mg in total; and/or
   d) about 2.6×10⁻⁸-5.1×10⁻⁸ mol/kg body weight, or about 2.0-4.1×10⁻⁶ mol in total.
More specifically, the amount of binding agent in Dose A may be
   a) 0.3-2.5 mg/kg body weight, or 25-200 mg in total; and/or
   b) 2.1×10⁻⁹-1.7×10⁻⁸ mol/kg body weight, or 1.7×10⁻⁷-1.4×10⁻⁶ mol in total; and
the amount of binding agent in Dose B may be
   c) 3.8-7.5 mg/kg body weight, or 300-600 mg in total; and/or
   d) 2.6×10⁻⁸-5.1×10⁻⁸ mol/kg body weight, or 2.0-4.1×10⁻⁶ mol in total.

The amount of said binding agent in Dose A may in particular be about 0.4-2.3 mg/kg body weight or about 30 to about 180 mg in total, and/or about 2.56×10⁻⁹ to about 1.53×10⁻⁸ mol/kg body weight or about 2.04×10⁻⁷ to about 1.23×10⁻⁶ mol in total.

The amount of said binding agent in Dose A may in particular be about 0.5 to about 2.0 mg/kg body weight or about 40 to about 160 mg in total, and/or about 3.41×10⁻⁹ to about 1.36×10⁻⁸ mol/kg body weight or about 2.73×10⁻⁷ to about 1.09×10⁻⁶ mol in total.

The amount of said binding agent in Dose A may in particular be about 0.6 to about 1.9 mg/kg body weight or about 50 to about 150 mg in total, and/or about 4.26×10⁻⁹ to about 1.28×10⁻⁸ mol/kg body weight or about 3.41×10⁻⁷ to about 1.02×10⁻⁶ mol in total.

The amount of said binding agent in Dose A may in particular be about 0.8 to about 1.8 mg/kg body weight or about 60 to about 140 mg in total, and/or about 5.11×10⁻⁹ to about 1.19×10⁻⁸ mol/kg body weight or about 4.09×10⁻⁷ to about 9.54×10⁻⁷ mol in total.

The amount of said binding agent in Dose A may in particular be about 0.9 to about 1.6 mg/kg body weight or about 70 to about 130 mg in total, and/or about 5.96×10⁻⁹ to about 1.11×10⁻⁸ mol/kg body weight or about 4.77×10⁻⁷ to about 8.86×10⁻⁷ mol in total.

The amount of said binding agent in Dose A may in particular be about 1 to about 1.5 mg/kg body weight or about 80 to about 120 mg in total, and/or about 6.81×10⁻⁹ to about 1.02×10⁻⁸ mol/kg body weight or about 5.45×10⁻⁷ to about 8.18×10⁻⁷ mol in total.

The amount of said binding agent in Dose A may in particular be about 1.1 to about 1.4 mg/kg body weight or about 90 to about 110 mg in total, and/or about 7.67×10⁻⁹ to about 9.37×10⁻⁹ mol/kg body weight or about 6.13×10⁻⁷ to about 7.49×10⁻⁷ mol in total.

The amount of said binding agent in Dose A may in particular be about 1.2 to about 1.3 mg/kg body weight or about 95 to about 105 mg in total, and/or about 8.09×10⁻⁹ to about 8.94×10⁻⁹ mol/kg body weight or about 6.47×10⁻⁷ to about 7.16×10⁻⁷ mol in total.

The amount of said binding agent in Dose A may in particular be 0.4-2.3 mg/kg body weight or 30-180 mg in total, and/or 2.56×10⁻⁹-1.53×10⁻⁸ mol/kg body weight or 2.04×10⁻⁷-1.23×10⁻⁶ mol in total.

The amount of said binding agent in Dose A may in particular be 0.5-2.0 mg/kg body weight or 40-160 mg in total, and/or 3.41×10⁻⁹-1.36×10⁻⁸ mol/kg body weight or 2.73×10⁻⁷-1.09×10⁻⁶ mol in total.

The amount of said binding agent in Dose A may in particular be 0.6-1.9 mg/kg body weight or 50-150 mg in total, and/or 4.26×10⁻⁹-1.28×10⁻⁸ mol/kg body weight or 3.41×10⁻⁷-1.02×10⁻⁶ mol in total.

The amount of said binding agent in Dose A may in particular be 0.8-1.8 mg/kg body weight or 60-140 mg in total, and/or 5.11×10⁻⁹-1.19×10⁻⁸ mol/kg body weight or 4.09×10⁻⁷-9.54×10⁻⁷ mol in total.

The amount of said binding agent in Dose A may in particular be 0.9-1.6 mg/kg body weight or 70-130 mg in total, and/or 5.96×10⁻⁹-1.11×10⁻⁸ mol/kg body weight or 4.77×10⁻⁷-8.86×10⁻⁷ mol in total.

The amount of said binding agent in Dose A may in particular be 1-1.5 mg/kg body weight or 80-120 mg in total, and/or 6.81×10⁻⁹-1.02×10⁻⁸ mol/kg body weight or 5.45×10⁻⁷-8.18×10⁻⁷ mol in total.

The amount of said binding agent in Dose A may in particular be 1.1-1.4 mg/kg body weight or 90-110 mg in total, and/or 7.67×10⁻⁹-9.37×10⁻⁹ mol/kg body weight or 6.13×10⁻⁷-7.49×10⁻⁷ mol in total.

The amount of said binding agent in Dose A may in particular be 1.2-1.3 mg/kg body weight or 95-105 mg in total, and/or 8.09×10⁻⁹-8.94×10⁻⁹ mol/kg body weight or 6.47×10⁻⁷-7.16×10⁻⁷ mol in total.

It is currently preferred that the amount of the binding agent in Dose A is
a) about 1.25 mg/kg body weight, or about 100 mg in total; and/or
b) about 8.5×10⁻⁹ mol/kg body weight, or about 6.8×10⁻⁷ mol in total.

It is further preferred that the amount of the binding agent in Dose A is
a) 1.25 mg/kg body weight, or 100 mg in total; and/or
b) 8.5×10⁻⁹ mol/kg body weight, or 6.8×10⁻⁷ mol in total.

The amount of binding agent in Dose B may in particular be about 4.4 to about 7.4 mg/kg body weight or 350 to about 590 mg in total, and/or about 2.98×10⁻⁸ to about 5.03×10⁻⁸ mol/kg body weight or about 2.39×10⁻⁶ to about 4.02×10⁻⁶ mol in total.

The amount of binding agent in Dose B may in particular be about 5.0 to about 7.25 mg/kg body weight or about 400 to about 580 mg in total, and/or about 3.41×10⁻⁸ to about 4.94×10⁻⁸ mol/kg body weight or about 2.73×10⁻⁶ to about 3.95×10⁻⁶ mol in total.

The amount of binding agent in Dose B may in particular be about 5.3 to about 7.1 mg/kg body weight or about 420 about 570 mg in total, and/or about 3.58×10⁻⁸ to about 4.86×10⁻⁸ mol/kg body weight or about 2.86×10⁻⁶ to about 3.88×10⁻⁶ mol in total. The amount of binding agent in Dose B may in particular be about 5.4 to about 7.0 mg/kg body weight or about 430 to about 560 mg in total, and/or about 3.66×10⁻⁸ to about 4.77×10⁻⁸ mol/kg body weight or about 2.93×10⁻⁶ to about 3.82×10⁻⁶ mol in total.

The amount of binding agent in Dose B may in particular be about 5.5 to about 6.9 mg/kg body weight or about 440 to about 550 mg in total, and/or about 3.75×10⁻⁸ to about 4.69×10⁻⁸ mol/kg body weight or about 3.00×10⁻⁶ to about 3.75×10⁻⁶ mol in total.

The amount of binding agent in Dose B may in particular be about 5.6 to about 6.8 mg/kg body weight or about 450 to about 540 mg in total, and/or about 3.83×10⁻⁸ to about 4.60×10⁻⁸ mol/kg body weight or about 3.07×10⁻⁶ to about 3.68×10⁻⁶ mol in total;

The amount of binding agent in Dose B may in particular be about 5.8 to about 6.6 mg/kg body weight or about 460 to about 530 mg in total, and/or about 3.92×10⁻⁸ to about 4.51×10⁻⁸ mol/kg body weight or about 3.13×10⁻⁶ to about 3.61×10⁻⁶ mol in total.

The amount of binding agent in Dose B may in particular be about 5.9 to about 6.5 mg/kg body weight or about 470 to about 520 mg in total, and/or about 4.00×10⁻⁸ to about 4.43×10⁻⁸ mol/kg body weight or about 3.20×10⁻⁶ to about 3.54×10⁻⁶ mol in total.

The amount of binding agent in Dose B may in particular be about 6.0 to about 6.4 mg/kg body weight or about 480 to about 515 mg in total, and/or about 4.09×10⁻⁸ to about 4.39×10⁻⁸ mol/kg body weight or about 3.27×10⁻⁶ to about 3.51×10⁻⁶ mol in total.

The amount of binding agent in Dose B may in particular be about 6.1 to about 6.4 mg/kg body weight or about 490 to about 510 mg in total, and/or about 4.17×10⁻⁸ to about 4.34×10⁻⁸ mol/kg body weight or about 3.34×10⁻⁶ to about 3.48×10⁻⁶ mol in total.

The amount of binding agent in Dose B may in particular be about 6.2 to about 6.3 mg/kg body weight or about 495 to about 505 mg in total, and/or about 4.22×10⁻⁸ to about 4.30×10⁻⁸ mol/kg body weight or about 3.37×10⁻⁶ to about 3.44×10⁻⁶ mol in total.

The amount of binding agent in Dose B may in particular be 4.4-7.4 mg/kg body weight or 350-590 mg in total, and/or 2.98×10⁻⁸-5.03×10⁻⁸ mol/kg body weight or 2.39×10⁻⁶-4.02×10⁻⁶ mol in total.

The amount of binding agent in Dose B may in particular be 5.0-7.25 mg/kg body weight or 400-580 mg in total, and/or 3.41×10⁻⁸-4.94×10⁻⁸ mol/kg body weight or 2.73×10⁻⁶-3.95×10⁻⁶ mol in total.

The amount of binding agent in Dose B may in particular be 5.3-7.1 mg/kg body weight or 420-570 mg in total, and/or 3.58×10⁻⁸-4.86×10⁻⁸ mol/kg body weight or 2.86×10⁻⁶-3.88×10⁻⁶ mol in total.

The amount of binding agent in Dose B may in particular be 5.4-7.0 mg/kg body weight or 430-560 mg in total, and/or 3.66×10⁻⁸-4.77×10⁻⁸ mol/kg body weight or 2.93×10⁻⁶-3.82×10⁻⁶ mol in total.

The amount of binding agent in Dose B may in particular be 5.5-6.9 mg/kg body weight or 440-550 mg in total, and/or 3.75×10⁻⁸-4.69×10⁻⁸ mol/kg body weight or 3.00×10⁻⁶-3.75×10⁻⁶ mol in total.

The amount of binding agent in Dose B may in particular be 5.6-6.8 mg/kg body weight or 450-540 mg in total, and/or 3.83×10⁻⁸-4.60 ×10⁻⁸ mol/kg body weight or 3.07×10⁻⁶-3.68×10⁻⁶ mol in total;

The amount of binding agent in Dose B may in particular be 5.8-6.6 mg/kg body weight or 460-530 mg in total, and/or 3.92×10⁻⁸-4.51×10⁻⁸ mol/kg body weight or 3.13×10⁻⁶-3.61×10⁻⁶ mol in total.

The amount of binding agent in Dose B may in particular be 5.9-6.5 mg/kg body weight or 470-520 mg in total, and/or 4.00×10⁻⁸-4.43×10⁻⁸ mol/kg body weight or 3.20×10⁻⁶-3.54×10⁻⁶ mol in total.

The amount of binding agent in Dose B may in particular be 6.0-6.4 mg/kg body weight or 480-515 mg in total, and/or 4.09×10⁻⁸-4.39×10⁻⁸ mol/kg body weight or 3.27×10⁻⁶-3.51×10⁻⁶ mol in total.

The amount of binding agent in Dose B may in particular be 6.1-6.4 mg/kg body weight or 490-510 mg in total, and/or 4.17×10⁻⁸-4.34×10⁻⁸ mol/kg body weight or 3.34×10⁻⁶-3.48×10⁻⁶ mol in total.

The amount of binding agent in Dose B may in particular be 6.2-6.3 mg/kg body weight or 495-505 mg in total, and/or 4.22×10⁻⁸-4.30×10⁻⁸ mol/kg body weight or 3.37×10⁻⁶-3.44×10⁻⁶ mol in total.

It is currently preferred that the amount of binding agent in Dose B is
a) about 6.25 mg/kg body weight, or about 500 mg in total; and/or
b) about 4.3×10⁻⁸ mol/kg body weight, or about 3.4×10⁻⁶ mol in total.

It is further preferred that the amount of binding agent in Dose B is
a) 6.25 mg/kg body weight, or 500 mg in total; and/or
b) 4.3×10⁻⁸ mol/kg body weight, or 3.4×10⁻⁶ mol in total.

It is further preferred that the dosing schedule comprises administration of Dose A in one or more treatment cycles, followed by administration of dose B in one or more treatment cycles.

Dose A may be administered once in each treatment cycles, such as on day 1 in each treatment cycle.

Also, Dose B may be administered once in each treatment cycles, such as on day 1 in each treatment cycle.

Dose A may be administered in one or more treatment cycles, each treatment cycle having a duration of three weeks/21 days, such as in 2, 3, 4 or 5 treatment cycles, each treatment cycle having a duration of three weeks/21 days. Preferably, Dose A is administered in two (2) treatment cycles, each treatment cycle having a duration of three weeks/21 days

Dose A is preferably administered once in each of said three-week/21-day treatment cycle (Q3W).

Dose A may in particular be administered on day 1 in each of said one or more three-week/21-day treatment cycles.

Dose B may be administered in one or more treatment cycles, each treatment cycle having a duration of 6-weeks/42-days. In particular, Dose B may be administered 2-5 treatment cycles, each treatment cycle having a duration of 6-weeks/42-days, such as in 2-10 treatment cycles, each treatment cycle having a duration of 6-weeks/42-days, such as in 2-20 treatment cycles, each treatment cycle having a duration of 6-weeks/42-days or such as in 2-50 treatment cycles, each treatment cycle having a duration of 6-weeks/42-days.

Dose B is preferably administered once in each of said one or more 6-week/42-day treatment cycles (Q6W).

In the method disclosed herein, Dose B may in particular be administered on day 1 in each of said one or more 6-week/42-day treatment cycles.

In further embodiments of the invention, the dosing schedule comprises administration of Dose A in two (2) treatment cycles, followed by administration of dose B in one or more treatment cycles.

In the context of the invention, Dose B may be considered “maintenance therapy” and may thus be continued until complete tumor regression or until disease progression. Hence, in the method according to the invention the said dosing schedule comprises administration of Dose A, followed by administration of dose B until complete tumor regression or disease progression.

Further to dosing the binding agent the method disclosed herein may comprise collecting whole blood samples and assessing PD-L1 receptor occupancy by the binding agent.

The binding agent is preferably administered by systemic administration, in particular administration by intravenous injection or infusion.

Each dose may be infused over a minimum of 30 minutes, such as over a minimum of 60 minutes, a minimum of 90 minutes, a minimum of 120 minutes or a minimum of 240 minutes.

The binding agent used in the presently disclosed method may be a binding agent, wherein
a) the first antigen-binding region may comprise a heavy chain variable region (VH) comprising the complementarity determining region 1 (CDR1), complementarity determining region 2 (CDR2), and complementarity determining region 3 (CDR3) sequences of SEQ ID NO: 1, and a light chain variable region (VL) comprising the complementarity determining region1 (CDR1), complementarity determining region 2 (CDR2), and complementarity determining region 3 (CDR3) sequences of SEQ ID NO: 5;
and
b) the second antigen-binding region may comprise a heavy chain variable region (VH) comprising the complementarity determining region 1 (CDR1), complementarity determining region 2 (CDR2), and complementarity determining region 3 (CDR3) sequences of SEQ ID NO: 8, and a light chain variable region (VL) comprising the complementarity determining region1 (CDR1), complementarity determining region 2 (CDR2), and complementarity determining region 3 (CDR3) sequences of SEQ ID NO: 12.

In particular, the binding agent may be a binding agent, wherein
a) the first antigen-binding region comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, GAS, 7, respectively;
and
b) the second antigen-binding region comprises a heavy chain variable region (VH) comprising the CDR1, CDR2, and CDR3 sequences set forth in: SEQ ID NO: 9, 10, 11 respectively, and a light chain variable region (VL) comprising the CDR1, CDR2, and CDR3 sequences set forth in: SEQ ID NO: 13, DDN, 14, respectively.

Each variable region may comprise three complementarity determining regions (CDR1, CDR2, and CDR3) and four framework regions (FR1, FR2, FR3, and FR4).

The complementarity determining regions and the framework regions are preferably arranged from amino-terminus to carboxy-terminus in the following order: FR1, CDR1, FR2, CDR2, FR3, CDR3, FR4.

The binding agent used according to the present disclosure may comprise a first and a second antigen-binding region, wherein
a) the first antigen-binding region comprises a heavy chain variable region (VH) comprising an amino acid sequence having at least 90%, at least 95%, at least 97%, at least 99%, or 100% sequence identity to SEQ ID NO: 1 and a light chain variable region (VL) region comprising an amino acid sequence having at least 90%, at least 95%, at least 97%, at least 99%, or 100% sequence identity to SEQ ID NO: 5;
and
b) the second antigen-binding region comprises a heavy chain variable region (VH) comprising an amino acid sequence having at least 90%, at least 95%, at least 97%, at least 99%, or 100% sequence identity to SEQ ID NO: 8 and a light chain variable region (VL) region comprising an amino acid sequence having at least 90%, at least 95%, at least 97%, at least 99%, or 100% sequence identity to SEQ ID NO: 12.

In particular, the binding agent used according to the present disclosure may comprise a first and a second antigen-binding region, wherein
a) the first antigen-binding region comprises a heavy chain variable region (VH) comprising the amino acid sequence set forth in SEQ ID NO: 1 and a light chain variable region (VL) region comprising the amino acid sequence set forth in SEQ ID NO: 5;
and
b) the second antigen-binding region comprises a heavy chain variable region (VH) comprising the amino acid sequence set forth in SEQ ID NO: 8 and a light chain variable region (VL) region comprising the amino acid sequence set forth in SEQ ID NO: 12.

The binding agent may in particular be an antibody. Examples of different classes of binding agents according to the present invention include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc regions or parts thereof; (v) Fab fusion molecules, wherein different Fab-fragments are fused together, fused to heavy-chain constant-domains, Fc regions or parts thereof; and (vi) ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule fused to heavy-chain constant-domains, Fc regions or parts thereof.

Examples of IgG-like molecules with complementary CH3 domain molecules include but are not limited to the Triomab/Quadroma molecules (Trion Pharma/Fresenius Biotech; Roche, WO2011069104), the so-called Knobs-into-Holes molecules (Genentech, WO9850431), CrossMAbs (Roche, WO2011117329) and the electrostatically-matched molecules (Amgen, EP1870459 and WO2009089004; Chugai, US201000155133; Oncomed, WO2010129304), the LUZ-Y molecules (Genentech, Wranik et al. J. Biol. Chem. 2012, 287(52): 43331-9, doi: 10.1074/jbc.M112.397869. Epub 2012 Nov 1), DIG-body and PIG-body molecules (Pharmabcine, WO2010134666, WO2014081202), the Strand Exchange Engineered Domain body (SEEDbody) molecules (EMD Serono, WO2007110205), the Biclonics molecules (Merus, WO2013157953), FcΔAdp molecules (Regeneron, WO201015792), bispecific IgG1 and IgG2 molecules (Pfizer/Rinat, WO11143545), Azymetric scaffold molecules (Zymeworks/Merck, WO2012058768), mAb-Fv molecules (Xencor, WO2011028952), bivalent bispecific antibodies (WO2009080254) and the DuoBody® molecules (Genmab, WO2011131746).

Examples of recombinant IgG-like dual targeting molecules include but are not limited to Dual Targeting (DT)-Ig molecules (WO2009058383), Two-in-one Antibody (Genentech; Bostrom, et al 2009. Science 323, 1610-1614.), Cross-linked Mabs (Karmanos Cancer Center), mAb2 (F-Star, WO2008003116), Zybody molecules (Zyngenia; LaFleur et al. MAbs. 2013 Mar-Apr;5(2):208-18), approaches with common light chain (Crucell/Merus, U.S. Pat. No. 7,262,028), κλBodies (NovImmune, WO2012023053) and CovX-body (CovX/Pfizer; Doppalapudi, V. R., et al 2007. Bioorg. Med. Chem. Lett. 17,501-506.).

Examples of IgG fusion molecules include but are not limited to Dual Variable Domain (DVD)-Ig molecules (Abbott, U.S. Pat. No. 7,612,181), Dual domain double head antibodies (Unilever; Sanofi Aventis, WO20100226923), IgG-like Bispecific molecules (ImClone/Eli Lilly, Lewis et al. Nat Biotechnol. 2014 Feb; 32(2): 191-8), Ts2Ab (MedImmune/AZ; Dimasi et al. J Mol Biol. 2009 Oct 30;393(3):672-92) and BsAb molecules (Zymogenetics, WO2010111625), HERCULES molecules (Biogen Idec, U.S. Pat. No. 007,951,918), scFv fusion molecules (Novartis), scFv fusion molecules (Changzhou Adam Biotech Inc, CN 102250246) and TvAb molecules (Roche, WO2012025525, WO2012025530).

Examples of Fc fusion molecules include but are not limited to ScFv/Fc Fusions (Pearce et al., Biochem Mol Biol Int. 1997 Sep;42(6):1179-88), SCORPION molecules (Emergent BioSolutions/Trubion, Blankenship J W, et al. AACR 100th Annual meeting 2009 (Abstract #5465); Zymogenetics/BMS, WO2010111625), Dual Affinity Retargeting Technology (Fc-DART) molecules (MacroGenics, WO2008157379, WO2010080538) and Dual(ScFv)2-Fab molecules (National Research Center for Antibody Medicine—China).

Examples of Fab fusion bispecific antibodies include but are not limited to F(ab)2 molecules (Medarex/AMGEN; Deo et al J Immunol. 1998 Feb 15;160(4): 1677-86.), Dual-Action or Bis-Fab molecules (Genentech, Bostrom, et al 2009. Science 323, 1610-1614.), Dock-and-Lock (DNL) molecules (ImmunoMedics, WO2003074569, WO2005004809), Bivalent Bispecific molecules (Biotecnolschoonjans, J Immunol. 2000 Dec 15;165(12):7050-7.) and Fab-Fv molecules (UCB-Celltech, WO 2009040562 A1).

Examples of ScFv-, diabody-based and domain antibodies include but are not limited to Bispecific T Cell Engager (BITE) molecules (Micromet, WO2005061547), Tandem Diabody molecules (TandAb) (Affimed) Le Gall et al., Protein Eng Des Sel. 2004 Apr; 17(4): 357-66.), Dual Affinity Retargeting Technology (DART) molecules (MacroGenics, WO2008157379, WO2010080538), Single-chain Diabody molecules (Lawrence, FEBS Lett. 1998 Apr 3;425(3):479-84), TCR-like Antibodies (AIT, ReceptorLogics), Human Serum Albumin ScFv Fusion (Merrimack, WO2010059315) and COMBODY molecules (Epigen Biotech, Zhu et al. Immunol Cell Biol. 2010 Aug;88(6): 667-75.), dual targeting nanobodies (Ablynx, Hmila et al., FASEB J. 2010) and dual targeting heavy chain only domain antibodies.

In currently preferred embodiments, the binding agent is a multispecific antibody, such as a bispecific antibody. In particular, the binding agent used according to the invention may have no more than two binding regions.

Many different formats and uses of bispecific antibodies are known in the art, and were reviewed by Kontermann; Drug Discov Today, 2015 Jul;20(7): 838-47 and; MAbs, 2012 Mar-Apr;4(2):182-97.

A bispecific antibody used according to the present invention is not limited to any particular bispecific format or method of producing it. Examples of bispecific antibody molecules which may be used in the present invention comprise (i) a single antibody that has two arms comprising different antigen-binding regions; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

In one embodiment, the binding agent used in the present invention is a diabody or a cross-body. In one embodiment, the binding agent of the invention is a bispecific antibody obtained via a controlled Fab-arm exchange (such as described in WO2011131746 (Genmab)).

Binding agents used according to the invention are preferably human, humanized or chimeric antibodies. In embodiments, wherein the antibody is a bispecific antibody, both half-molecules can be human, humanized or chimeric, or the half-molecules can differ in character with respect to sequence origin.

For example, in one embodiment, the binding agent, e.g. a bispecific antibody, comprises two half-molecules each comprising an antigen-binding region, wherein
(i) the half-molecule(s) comprising the antigen-binding region capable of binding to human PD-L1 is/are chimeric, and/or
(ii) the half-molecule comprising the antigen-binding region capable of binding to human CD137, if present, is chimeric.

For example, in another embodiment, the bispecific antibody comprises two half-molecules each comprising an antigen-binding region, wherein
(i) the half-molecule(s) comprising the antigen-binding region capable of binding to human PD-L1 is/are humanized, and/or
(ii) the half-molecule comprising the antigen-binding region capable of binding to human CD137, if present, is humanized.

For example, the bispecific antibody comprises two half-molecules may each comprises an antigen-binding region, wherein
(i) the half-molecule(s) comprising the antigen-binding region capable of binding to human PD-L1 is/are human, and/or
(ii) the half-molecule comprising the antigen-binding region capable of binding to human CD137, is human.

Thus, for example, the antigen-binding region(s) capable of binding to human PD-L1 may be humanized, and the antigen-binding region capable of binding to human CD137, may be humanized.

Alternatively, the antigen-binding region(s) capable of binding to human PD-L1 may be human, and the antigen-binding region capable of binding to human CD137, may be human.

The binding agent may be a bispecific antibody comprising an antigen-binding region capable of binding to human PD-L1 and an antigen-binding region capable of binding to human CD137, wherein the half-molecule comprising the antigen-binding region capable of binding to human PD-L1 is human, humanized or chimeric, and the half-molecule comprising the antigen-binding region capable of binding to human CD137 is humanized.

Preferably, the half-molecule comprising the antigen-binding region capable of binding to human PD-L1 is human and the half-molecule comprising the antigen-binding region capable of binding to human CD137 is humanized.

In the method disclosed herein, the binding agent may be in the format of a full-length antibody or an antibody fragment.

In the method according to the present disclosure, the binding agent may comprise
i) a polypeptide comprising, consisting of or consisting essentially of, said first heavy chain variable region (VH) and a first heavy chain constant region (CH), and
ii) a polypeptide comprising, consisting of or consisting essentially of, said second heavy chain variable region (VH) and a second heavy chain constant region (CH).

In the method according to the present disclosure, the binding agent may further comprise
i) a polypeptide comprising said first light chain variable region (VL) and further comprising a first light chain constant region (CL), and
ii) a polypeptide comprising said second light chain variable region (VL) and further comprising a second light chain constant region (CL).

The binding agent may be an antibody comprising a first binding arm and a second binding arm, wherein the first binding arm comprises
i) a polypeptide comprising said first heavy chain variable region (VH) and said first heavy chain constant region (CH), and
ii) a polypeptide comprising said first light chain variable region (VL) and said first light chain constant region (CL);
   and the second binding arm comprises
iii) a polypeptide comprising said second heavy chain variable region (VH) and said second heavy chain constant region (CH), and
iv) a polypeptide comprising said second light chain variable region (VL) and said second light chain constant region (CL).

Each of the first and second heavy chain constant regions (CH) may comprise one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

Each of the first and second heavy chain constant regions (CHs) may comprise a CH3 region and each of the CH3 regions, or both CH3 regions, comprise asymmetrical mutations.

The bispecific antibody used according to the invention may comprise a first Fc sequence comprising a first CH3 region, and a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions. More details on these interactions and how they can be achieved are provided in WO2011131746 and WO2013060867 (Genmab), which are hereby incorporated by reference.

A stable bispecific PD-L1xCD137 antibody can be obtained at high yield using a particular method on the basis of one homodimeric starting PD-L1 antibody and one homodimeric starting CD137 antibody containing only a few, conservative, asymmetrical mutations in the CH3 regions. Asymmetrical mutations mean that the sequences of said first and second CH3 regions contain amino acid substitutions at non-identical positions.

In the method according to the present disclosure, the binding agent may be a binding agent which comprises a first and second constant region (CH), wherein in the first CH at least one of the amino acids in a position corresponding to a position selected from the group consisting of T366, L368, K370, D399, F405, Y407, and K409 in a human IgG1 heavy chain according to EU numbering has been substituted, and wherein in said second CH at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.

In the method according to the present disclosure, the binding agent may be a binding agent, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

In the method according to the present invention the binding agent preferably induces Fc-mediated effector function to a lesser extent compared to another antibody comprising the same first and second antigen binding regions and two heavy chain constant regions (CHs) comprising human IgG1 hinge, CH2 and CH3 regions.

The first and second heavy chain constant regions (CHs) may be modified so that the antibody induces Fc-mediated effector function to a lesser extent compared to an antibody which is identical except for comprising non-modified first and second heavy chain constant regions (CHs).

Each of said non-modified first and second heavy chain constant regions (CHs) may comprise the amino acid sequence set forth in SEQ ID NO: 15 or SEQ ID NO: 30.

Said Fc-mediated effector function may be measured by binding to Fcγ receptors, binding to C1q, or induction of Fc-mediated cross-linking of Fcγ receptors.

The Fc-mediated effector function may be measured by determining binding to C1q.

The first and second heavy chain constant regions may have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

In the method according to the invention, it is preferred that at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

Preferably, the amino acids residues at the positions corresponding to positions L234 and L235 in a human IgG1 heavy chain according to EU numbering are F and E, respectively, in said first and second heavy chains.

It is further preferred that the amino acids residues at the positions corresponding to positions L234, L235, and D265 in a human IgG1 heavy chain according to EU numbering are F, E, and A, respectively, in said first and second heavy chain constant regions (HCs).

The method according to the present disclosure may use a binding agent wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

In the binding agent used in the method provided herein is a binding agent wherein the amino acid residues at the positions corresponding to positions L234, L235, and D265 in a human IgG1 heavy chain according to EU numbering of both the first and second heavy chain constant regions are F, E, and A, respectively, and wherein (i) the amino acid at the position corresponding to F405 in a human IgG1 heavy chain according to EU numbering of the first heavy chain constant region is L, and the amino acid residue at the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

Binding agents having the combination of three amino acid substitutions L234F, L235E and D265A and in addition the K409R or the F405L mutation are referred to herein with the suffix “FEAR” or “FEAL”, respectively.

In the binding agent, the constant region of said first and/or second heavy chain may comprise, may consist essentially of or may consist of an amino acid sequence selected from the group consisting of
a) the sequence set forth in SEQ ID NO: 15 or SEQ ID NO: 30 [IgG1-FC],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4, at the most 3, at the most 2 or at the most 1 substitution compared to the amino acid sequence defined in a) or b).

In the binding agent used according to the invention, the constant region of said first or second heavy chain, such as the first heavy chain, may comprise, may consist essentially of or may consist of an amino acid sequence selected from the group consisting of
a) the sequence set forth in SEQ ID NO: 16 or SEQ ID NO: 31 [IgG1-F405L],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 9 substitutions, such as at the most 8, at the most 7, at the most 6, at the most 5, at the most 4, at the most 3, at the most 2 or at the most 1 substitution compared to the amino acid sequence defined in a) or b).

The constant region of said first or second heavy chain, such as the constant region of the second heavy chain, may comprise, may consist essentially of or may consist of an amino acid sequence selected from the group consisting of
a) the sequence set forth in SEQ ID NO: 17 or SEQ ID NO: 32 [IgG1-F409R]
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution compared to the amino acid sequence defined in a) or b).

Further, the constant region of said first and/or second heavy chain, may comprise or consist essentially of, or may consist of, an amino acid sequence selected from the group consisting of
a) the sequence set forth in SEQ ID NO: 18 or SEQ ID NO: 33 [IgG1-Fc_FEA],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 7 substitutions, such as at the most 6 substitutions, at the most 5, at the most 4, at the most 3, at the most 2 or at the most 1 substitution compared to the amino acid sequence defined in a) or b).

The constant region of said first and/or second heavy chain, such as of the first heavy chain, may comprise or consist essentially of or may consist of an amino acid sequence selected from the group consisting of
a) the sequence set forth in SEQ ID NO: 19 or SEQ ID NO: 34 [IgG1-Fc_FEAL],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4 substitutions, at the most 3, at the most 2 or at the most 1 substitution compared to the amino acid sequence defined in a) or b).

The constant region of said first and/or second heavy chain, such as the constant region of the second heavy chain, may comprise or may consist essentially of or may consist of an amino acid sequence selected from the group consisting of
a) the sequence set forth in SEQ ID NO: 20 or SEQ ID NO: 35 [IgG1-Fc_FEAR]
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4, at the most 3, at the most 2 or at the most 1 substitution compared to the amino acid sequence defined in a) or b).

The constant region sequences listed in SEQ ID NOs: 15-20 list a terminal lysine (K), whereas the C-terminal lysine is omitted from the sequences set forth in SEQ ID NOS: 30-36, 38. The origin of this lysine is a naturally occurring sequence found in humans from which these Fc regions are derived. During cell culture production of recombinant antibodies, this terminal lysine can be cleaved off by proteolysis by endogenous carboxypeptidase(s), resulting in a constant region having the same sequence but lacking the C-terminal lysine. For manufacturing purposes of antibodies, the DNA encoding this terminal lysine can be omitted from the sequence such that antibodies are produced without the lysine. Antibodies produced from nucleic acid sequences that either do, or do not encode a terminal lysine are substantially identical in sequence and in function since the degree of processing of the terminal lysine is typically high when e.g. using antibodies produced in CHO-based production systems (Dick, L. W. et al. Biotechnol. Bioeng. 2008; 100: 1132-1143). Hence, it is understood that antibodies in accordance with the invention can be generated without encoding or having a terminal lysine such as listed herein. For manufacturing purposes, antibodies can thus be generated without having a terminal lysine.

Either of the human light chain constant regions, kappa (κ) or lambda (λ), may be used. Hence, in certain embodiments the binding agent disclosed herein may comprise a kappa (κ) light chain constant region.

Alternatively or additionally, the binding agent disclosed herein may comprise a lambda (λ) light chain constant region.

In the binding agent used according to the invention the first light chain constant region may be a kappa (κ) light chain constant region.

In the binding agent used according to the invention the second light chain constant region may be a lambda (λ) light chain constant region.

Alternatively, the binding agent used according to the invention may comprise a first light chain constant region, which is a lambda (λ) light chain constant region.

In the binding agent used according to the invention said second light chain constant region may be a kappa (κ) light chain constant region.

In certain embodiments the kappa (κ) light chain comprises an amino acid sequence selected from the group consisting of
a) the sequence set forth in SEQ ID NO: 21,
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

The lambda (λ) light chain may comprise an amino acid sequence selected from the group consisting of
a) the sequence set forth in SEQ ID NO: 22,
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

The binding agent used according in the method of the present disclosure may be of an isotype selected from the group consisting of IgG1, IgG2, IgG3, and IgG4.

The choice of isotype typically will be guided by the desired Fc-mediated effector functions, such as ADCC induction, or the requirement for an antibody devoid of Fc-mediated effector function (“inert” antibody). Exemplary isotypes are IgG1, IgG2, IgG3, and IgG4. The effector function of the antibodies of the present invention may be changed by isotype switching to, e.g., an IgG1, IgG2, IgG3, IgG4, IgD, IgA, IgE, or IgM antibody for various therapeutic uses.

In embodiments which are currently preferred, the binding agent is a full-length IgG1 antibody.

The binding agent used in the method according to the present disclosure may be an antibody, which is of the IgG1m(f) allotype. Alternatively, the antibody may be of the IgG1m(za) allotype.

The binding agent used according to the present disclosure may comprise
(i) a first binding arm comprising a first heavy chain variable region (VH) and a first light chain variable region (VL),
   wherein the first VH comprises a first HCDR1 sequence, a first HCDR2 sequence, and a first HCDR3 sequence, wherein the first HCDR1 sequence is set forth in SEQ ID NO: 2, wherein the first HCDR2 sequence is set forth in SEQ ID NO: 3, and wherein the first HCDR3 sequence comprises is set forth in SEQ ID NO: 4; and wherein the first VL comprises a first LCDR1 sequence, a first LCDR2 sequence, and a first LCDR3 sequence, wherein the first LCDR1 sequence is set forth in SEQ ID NO: 6, wherein the first LCDR2 sequence is GAS, and wherein the first LCDR3 sequence is set forth in SEQ ID NO: 7; and
(ii) a second binding arm comprising a second heavy chain variable region (VH) and a second light chain variable region (VL),
   wherein the second VH comprises a second HCDR1 sequence, a second HCDR2 sequence, and a second HCDR3 sequence, wherein the second HCDR1 sequence is set forth in SEQ ID NO: 9, wherein the second HCDR2 sequence is set forth in SEQ ID NO: 10, and wherein the second HCDR3 sequence is set forth in SEQ ID NO: 11; and wherein the second VL comprises a second LCDR1 sequence, a second LCDR2 sequence, and a second LCDR3 sequence, wherein the second LCDR1 sequence is set forth in SEQ ID NO: 13, wherein the second LCDR2 sequence is DDN, and wherein the second LCDR3 sequence is set forth in SEQ ID NO: 14;
      wherein the first binding arm comprises a first heavy chain constant region (CH) and the second binding arm comprises a second CH, wherein positions L234, L235, and D265 in a human IgGI heavy chain according to EU numbering are F, E, and A, respectively, in the first CH and the second CH; and
      wherein the amino acid in the position corresponding to F405 in a human IgGI heavy chain according to EU numbering is L in the first CH and the amino acid in the position corresponding to K409 in a human IgGI heavy chain according to EU numbering is R in the second CH.

The binding agent used according to the present disclosure may comprise
(i) a first binding arm comprising a first heavy chain variable region (VH) and a first light chain variable region (VL),
   wherein the first VH comprises a first HCDR1 sequence, a first HCDR2 sequence, and a first HCDR3 sequence, wherein the first HCDR1 sequence is set forth in SEQ ID NO: 2, wherein the first HCDR2 sequence is set forth in SEQ ID NO: 3, and wherein the first HCDR3 sequence is set forth in SEQ ID NO: 4, and wherein the first VL comprises a first LCDR1 sequence, a first LCDR2 sequence, and a first LCDR3 sequence, wherein the first LCDR1 sequence is set forth in SEQ ID NO: 6, wherein the first LCDR2 sequence is GAS, and wherein the first LCDR3 sequence is set forth in SEQ ID NO: 7; and
(ii) a second binding arm comprising a second heavy chain variable region (VH) and a second light chain variable region (VL), wherein the second VH comprises a second HCDR1 sequence, a second HCDR2 sequence, and a second HCDR3 sequence, wherein the second HCDR1 sequence is set forth in SEQ ID NO: 9, wherein the second HCDR2 sequence is set forth in SEQ ID NO: 10, and wherein the second HCDR3 sequence is set forth in SEQ ID NO: 11, and wherein the second VL comprises a second LCDR1 sequence, a second LCDR2 sequence, and a second LCDR3 sequence, wherein the second LCDR1 sequence is set forth in SEQ ID NO: 13, wherein the second LCDR2 sequence is DDN, and wherein the second LCDR3 sequence is set forth in SEQ ID NO: 14;
   wherein the first binding arm comprises a first heavy chain constant region (CH) and the second binding arm comprises a second CH, wherein positions L234, L235, and D265 in a human IgGI heavy chain according to EU numbering are F, E, and A, respectively, in the first CH and the second CH; and
   wherein the amino acid in the position corresponding to K409 in a human IgGI heavy chain according to EU numbering is R in the first CH and the amino acid in the position corresponding to F405 in a human IgGI heavy chain according to EU numbering is L in the second CH.

The binding agent used according to the present disclosure may comprise
(i) a first binding arm comprising a first heavy chain variable region (VH) and a first light chain variable region (VL),
   wherein the first VH comprises a first HCDR1 sequence, a first HCDR2 sequence, and a first HCDR3 sequence, wherein the first HCDR1 sequence comprises an amino acid sequence as set forth in SEQ ID NO: 9, wherein the first HCDR2 sequence comprises an amino acid sequence as set forth in SEQ ID NO: 10, and wherein the first HCDR3 sequence comprises an amino acid sequence as set forth in SEQ ID NO: 11, and wherein the first VL comprises a first LCDR1 sequence, a first LCDR2 sequence, and a first LCDR3 sequence, wherein the first LCDR1 sequence comprises an amino acid sequence as set forth in SEQ ID NO: 13, wherein the first LCDR2 sequence comprises the amino acid sequence GAS, and wherein the first LCDR3 sequence comprises an amino acid sequence as set forth in SEQ ID NO: 14; and
(ii) a second binding arm comprising a second heavy chain variable region (VH) and a second light chain variable region (VL),
   wherein the second VH comprises a second HCDR1 sequence, a second HCDR2 sequence, and a second HCDR3 sequence, wherein the second HCDR1 sequence comprises an amino acid sequence as set forth in SEQ ID NO: 18, wherein the second HCDR2 sequence comprises an amino acid sequence as set forth in SEQ ID NO: 19, and wherein the second HCDR3 sequence comprises an amino acid sequence as set forth in SEQ ID NO: 20, and wherein the second VL comprises a second LCDR1 sequence, a second LCDR2 sequence, and a second LCDR3 sequence, wherein the second LCDR1 sequence comprises an amino acid sequence as set forth in SEQ ID NO: 22, wherein the second LCDR2 sequence comprises the amino acid sequence DDN, and wherein the second LCDR3 sequence comprises an amino acid sequence as set forth in SEQ ID NO: 23;
      wherein the first binding arm comprises a first heavy chain constant region (CH) and the second binding arm comprises a second CH, wherein positions L234, L235, and D265 in a human IgGI heavy chain according to EU numbering are F, E, and A, respectively, in the first CH and the second CH; and
      wherein the amino acid in the position corresponding to F405 in a human IgGI heavy chain according to EU numbering is L in the first CH and the amino acid in the position corresponding to K409 in a human IgG1 heavy chain according to EU numbering is R in the second CH.

The binding agent used according to the present disclosure may comprise
(i) a first binding arm comprising a first heavy chain variable region (VH) comprising the amino acid sequence as set forth in SEQ ID NO: 1, and a first light chain variable region (VL) comprising the amino acid sequence as set forth in SEQ ID NO: 5; and
(ii) a second binding arm comprising a second heavy chain variable region (VH) comprising the amino acid sequence as set forth in SEQ ID NO: 8, and second light chain variable region (VL) comprising the amino acid sequence as set forth in SEQ ID NO: 12;
   wherein the first binding region comprises a first heavy chain constant region (CH) and the second binding arm comprises a second CH, wherein positions L234, L235, and D265 in a human IgG1 heavy chain according to EU numbering are F, E, and A, respectively, in the first CH and the second CH; and
   wherein the amino acid in the position corresponding to K409 in a human IgGI heavy chain according to EU numbering is R in the first CH and the amino acid in the position corresponding to F405 in a human IgGI heavy chain according to EU numbering is L in the second CH.

The binding agent used according to the present disclosure may comprise
(i) a first binding arm comprising a first heavy chain variable region (VH) comprising the amino acid sequence as set forth in SEQ ID NO: 1, and a first light chain variable region (VL) comprising the amino acid sequence as set forth in SEQ ID NO: 5; and
(ii) a second binding arm comprising a second heavy chain variable region (VH) comprising the amino acid sequence as set forth in SEQ ID NO: 8, and second light chain variable region (VL) comprising the amino acid sequence as set forth in SEQ ID NO: 12;
   wherein the first binding arm comprises a first heavy chain constant region (CH) and the second antigen binding region comprises a second CH, wherein positions L234, L235, and D265 in a human IgGI heavy chain according to EU numbering are F, E, and A, respectively, in the first CH and the second CH; and
   wherein the amino acid in the position corresponding to F405 in a human IgGI heavy chain according to EU numbering is L in the first CH and the amino acid in the position corresponding to K409 in a human IgGI heavy chain according to EU numbering is R in the second CH.

The binding agent used according to the present disclosure may comprise
i) A first heavy chain comprising, consisting essentially of or consisting of the amino acid sequence as set forth in SEQ ID NO: 36, and a first light chain comprising, consisting essentially of or consisting of the amino acid sequence as set forth in SEQ ID NO: 37; and
ii) a second heavy chain comprising, consisting essentially of or consisting of the amino acid sequence as set forth in SEQ ID NO: 38, and a second light chain comprising, consisting essentially of or consisting of the amino acid sequence as set forth in SEQ ID NO: 39.

The binding agent used according to the present disclosure may be in the format of a full-length antibody or an antibody fragment.

The binding agent used according to the present disclosure may be acasunlimab or a biosimilar thereof.

The binding agent used according to the invention may be in a pharmaceutically acceptable composition or formulation, such as a composition or formulation comprising histidine, sucrose and Polysorbate-80, which has a pH from 5 to 6.

In particular, the binding agent used according to the invention may be in a composition or formulation comprising about 20 mM histidine, about 250 mM Sucrose, about 0.02% Polysorbate-80, and having a pH of about 5.5.

The method according to any one of the preceding claims wherein the binding agent is in a composition or formulation comprising 10-30 mg binding agent/mL, such as 20 mg binding agent/mL.

When used in the method according to the present disclosure, the binding agent may be diluted prior to administration to the subject. The dilution may be in saline; e.g. 0.9% NaCl. The binding agent may be in a composition as defined above and may be diluted in 0.9% NaCl (saline) prior to administration.

The subject receiving treatment as disclosed herein may in particular be a human subject.

The tumor or cancer to be treated according to the present disclosure may be a solid tumor.

In certain embodiments the tumor is a PD-L1 positive tumor; i.e. a tumor that expresses PD-L1.

The tumor or cancer may be selected from the group consisting of melanoma, ovarian cancer, lung cancer (e.g. non-small cell lung cancer (NSCLC), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, prostate cancer, penile cancer, cervical cancer, Hodgkin's lymphoma, non-Hodgkin's lymphoma, Merkel cell carcinoma and mesothelioma.

The tumor or cancer may be selected from the group consisting of lung cancer (e.g. non-small cell lung cancer (NSCLC), urothelial cancer (cancer of the bladder, ureter, urethra, or renal pelvis), endometrial cancer (EC), breast cancer (e.g. triple negative breast cancer (TNBC)), squamous cell carcinoma of the head and neck (SCCHN) (e.g. cancer of the oral cavity, pharynx or larynx) and cervical cancer.

In currently preferred embodiments, the tumor or cancer is a lung cancer.

The lung cancer may in particular be a non-small cell lung cancer (NSCLC), such as a squamous or non-squamous NSCLC.

In certain embodiments the NSCLC does not have an epidermal growth factor (EGFR)-sensitizing mutation and/or anaplastic lymphoma (ALK) translocation/ROS1 rearrangement.

Lung cancer is the most common malignancy and the most common cause of cancer death worldwide. Non-small cell lung cancer (NSCLC) accounts for 85-90% of all lung cancer cases (Jemal et al., 2011). The five-year survival rate for NSCLC is approximately 18% (SEER, 2018). Major histological subtypes of NSCLC include adenocarcinoma, squamous cell carcinoma, adenosquamous carcinoma, large cell carcinoma, carcinoid tumors, and other less common subtypes, with adenocarcinoma being the most common.

Standard of care for patients with advanced or metastatic NSCLC who have progressed on targeted therapy or are no longer candidates for targeted therapy typically includes platinum-based chemotherapy. Platinum combinations have generated an overall response rate (ORR) of approximately 25-35%, a time to progression (TTP) of 4-6 months, and median survival of 8-10 months.

Tumor gene mutations/alterations have been identified and have impact on therapy selection. Identification of specific mutations or alterations in genes within the tumor, such as anaplastic lymphoma kinase (ALK), epidermal growth factor receptor (EGFR), c-ROS oncogene 1 (ROS1), BRAF, KRAS, and program death ligand-1 (PD-L1), aids the selection of potentially efficacious targeted therapies, while avoiding the use of therapies unlikely to provide clinical benefit (NCCN, 2018c). Activating sensitizing EGFR mutations are predictive for response to the EGFR Tyrosine Kinase Inhibitors (TKIs) (e.g., gefitinib, erlotinib, afatinib, and osimertinib). Similarly, TKIs (e.g., alectinib, ceritinib, and crizotinib) are effective therapies for ALK and ROS1 mutations and are also approved as first-line therapy for the respective mutations. Checkpoint inhibitor antibodies (e.g., pembrolizumab and nivolumab) that block the PD 1 and PD-L1 interaction have also been shown as effective treatment alone or in combination with chemotherapy for the treatment of patients with advanced or metastatic NSCLC whose tumors express PD-L1.

Despite multiple treatment options, patients with stage IV NSCLC ultimately have a poor prognosis and lung cancer remains the leading cause of cancer death for both men and women. The treatment rate diminishes with each line of therapy, as patients succumb to their cancer or experience deterioration of their health that makes further treatment impossible.

The lung cancer may be NSCLC, which does not have an epidermal growth factor (EGFR)-sensitizing mutation and/or anaplastic lymphoma (ALK) translocation/ROS1 rearrangement. EGFR sensitizing mutations refers to mutations that confer sensitivity to EGFR tyrosine kinase inhibitors (TKIs), such as approved tyrosine kinase inhibitors erlotinib, osimertinib, gefintinib, olmutinib, nazartinib and avitinib.

The epidermal growth factor receptor (EGFR) amino acid sequence is provided herein as SEQ ID NO: 27.

The subject receiving treatment as disclosed herein may have received prior treatment to reduce or preventing progression of said tumor or prior treating of said cancer. The subject may have received one, two, three or four prior systemic treatment regimens, such as for advanced/metastatic disease, and has experienced disease progression on or after last prior systemic treatment, such as disease progression determined by radiography.

In particular embodiments the treatment according to the invention is provided to a subject having received prior treatment; e.g. as defined above, wherein the last prior treatment was with a PD1 inhibitor or PD-L1 inhibitor, such as an anti PD-1 antibody or an anti-PD-L1 antibody, the PD-1 inhibitor or PD-L1 inhibitor being administered as monotherapy or as part of a combination therapy. It will be understood that prior treatment in the present context does not comprise treatment with a multispecific agent targeting PD-L1 and 4-1BB.

Preferably, the therapy according to the invention is provided to a subject when the time from progression of that subject on last treatment with a PD1 inhibitor or PD-L1 inhibitor, such as an anti PD-1 antibody or an anti-PD-L1 antibody is 8 months or less, such as 7 months or less, 6 months or less, 5 months or less, 4 months or less, 3 months or less, 2 months or less, 1 month or less, 3 weeks or less or such as 2 weeks or less.

By analogy, it may be preferred to offer therapy according to the present invention to a subjects when the time from last dosing of a PD1 inhibitor or PD-L1 inhibitor, such as an anti PD-1 antibody or an anti-PD-L1 antibody as part of last prior treatment is 8 months or less, such as 7 months or less, 6 months or less, 5 months or less, 4 months or less, 3 months or less, 2 months or less, 1 month or less, 3 weeks or less or such as 2 weeks or less.

The subject has received prior treatment in the form of platinum-based chemotherapy.

The subject receiving treatment as disclosed herein may be a subject that is not eligible for platinum-based therapy and has received prior treatment in the form of alternative chemotherapy, e.g. a treatment with gemcitabine-containing regimen.

The subject receiving treatment as disclosed herein may have received prior treatment with checkpoint inhibitor(s), such as agent(s) targeting PD-1/PD-L, such as a PD-1/PD-L1 inhibitor.

The subject may have experienced disease progression on or after treatment with checkpoint inhibitor(s), such as agent(s) targeting PD-1/PD-L, such as a PD-1/PD-L1 inhibitor.

The subject treated according to the present disclosure may have experienced disease progression on or after last prior treatment with checkpoint inhibitor(s), such as agent(s) targeting PD-1/PD-L, such as a PD-1/PD-L1 inhibitor.

The subject treated as disclosed herein may have experienced disease progression on or after last prior systemic treatment, such as disease progression determined by radiography.

In other embodiments, the subject has not received prior treatment with checkpoint inhibitor(s), such as agent(s) targeting PD-1/PD-L, such as a PD-1/PD-L1 inhibitor.

The method disclosed herein may be for first line treatment of said tumor or cancer.

Alternatively, the method may be for second line treatment of said tumor or cancer.

In a further aspect the present invention provides a binding agent for use in reducing or preventing progression of a tumor or for use in treatment of cancer, wherein the binding agent comprises a first antigen-binding region binding to human CD137, such as human CD137 consisting of the amino acid sequence set forth in SEQ ID NO: 24, and a second antigen-binding region binding to human PD-L1, such as human PD-L1 consisting of the amino acid sequence set forth in SEQ ID NO: 26, and the binding agent is administered to the subject in a dosing schedule that comprises administration of Dose A in one or more treatment cycles and administration of dose B in one or more treatment cycles,
the amount of binding agent in Dose A being
   c) about 0.3-2.5 mg/kg body weight, or about 25-200 mg in total; and/or
   d) about 2.1×10⁻⁹-1.7×10⁻⁸ mol/kg body weight, or about 1.7×10⁻⁷-1.4×10⁻⁶ mol in total; and
the amount of binding agent in Dose B being
   e) about 3.8 - 7.5 mg/kg body weight, or about 300-600 mg in total; and/or
   f) about 2.6×10⁻⁸-5.1×10⁻⁸ mol/kg body weight, or about 2.0-4.1×10⁻⁶ mol in total.

It will be understood that the features described above in relation to the method of the disclosure also apply in connection with the binding agent for use in reducing or preventing progression of a tumor or for use in treatment of cancer. In particular, the dosing schedule may be as further defined above.

Also, features of the binding agent disclosed above may apply in connection with the with the binding agent for use in reducing or preventing progression of a tumor or for use in treatment of cancer; e.g. amino acid sequences of the CDRs and variable regions as well as of the constant regions may be as defined above.

TABLE 3
SEQUENCES

| SEQ ID | NAME | SEQUENCE |
|---|---|---|
| 1 | VH_CD137-009-H7 | EVQLVESGGGLVQPGRSLRLSCTASGFSLNDYWMSWVRQAPGK |
| | | GLEWVGYIDVGGSLYYAASVKGRFTISRDDSKSIAYLQMNSLKTED |
| | | TAVYYCARGGLTYGFDLWGQGTLVTVSS |
| 2 | VH_CD137-009-H7_CDR1 | GFSLNDYW |
| 3 | VH_CD137-009-H7_CDR2 | IDVGGSL |
| 4 | VH_CD137-009-H7_CDR3 | ARGGLTYGFDL |
| 5 | VL_CD137-009-L2 | DIVMTQSPSSLSASVGDRVTITCQASEDISSYLAWYQQKPGKAPK |
| | | RLIYGASDLASGVPSRFSASGSGTDYTFTISSLQPEDIATYYCHYYAT |
| | | ISGLGVAFGGGTKVEIK |
| 6 | VL_CD137-009-L2_CDR1 | EDISSY |
| | VL_CD137-009-L2_CDR2 | GAS |
| 7 | VL_CD137-009-L2_CDR3 | HYYATISGLGVA |
| 8 | VH-PD-L1-547 | EVQLLEPGGGLVQPGGSLRLSCEASGSTFSTYAMSWVRQAPGKG |
| | | LEWVSGFSGSGGFTFYADSVRGRFTISRDSSKNTLFLQMSSLRAED |
| | | TAVYYCAIPARGYNYGSFQHWGQGTLVTVSS |
| 9 | VH-PD-L1-547-CDR1 | GSTFSTYA |
| 10 | VH-PD-L1-547-CDR2 | FSGSGGFT |
| 11 | VH-PD-L1-547-CDR3 | AIPARGYNYGSFQH |
| 12 | VL-PD-L1-547 | SYVLTQPPSVSVAPGQTARITCGGNNIGSKSVHWYQQKPGQAPV |
| | | LVVYDDNDRPSGLPERFSGSNSGNTATLTISRVEAGDEADYYCQV |
| | | WDSSSDHVVFGGGTKLTVL |
| 13 | VL-PD-L1-547-CDR1 | NIGSKS |
| | VL-PD-L1-547-CDR2 | DDN |
| 14 | VL-PD-L1-547-CDR3 | QVWDSSSDHVV |
| 15 | IgG1-Fc | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFFLYSKLTVDKSRWQQGNVFSCSVMHEALH |
| | | NHYTQKSLSLSPGK |
| 16 | IgG1-Fc_F405L | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFLLYSKLTVDKSRWQQGNVFSCSVMHEALH |
| | | NHYTQKSLSLSPGK |
| 17 | IgG1-Fc_K409R | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFFLYSRLTVDKSRWQQGNVFSCSVMHEALH |
| | | NHYTQKSLSLSPGK |
| 18 | IgG1-Fc_FEA | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKRVEPKSCDKTHTCPPCPAPEFEGGPSVFLFPPKPKDTLMISR |
| | | TPEVTCVVVAVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFFLYSKLTVDKSRWQQGNVFSCSVMHEALH |
| | | NHYTQKSLSLSPGK |
| 19 | IgG1-FEAR-Fc | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKRVEPKSCDKTHTCPPCPAPEFEGGPSVFLFPPKPKDTLMISR |
| | | TPEVTCVVVAVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFFLYSRLTVDKSRWQQGNVFSCSVMHEALH |
| | | NHYTQKSLSLSPGK |
| 20 | IgG1-FEAL-Fc | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKRVEPKSCDKTHTCPPCPAPEFEGGPSVFLFPPKPKDTLMISR |
| | | TPEVTCVVVAVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFLLYSKLTVDKSRWQQGNVFSCSVMHEALH |
| | | NHYTQKSLSLSPGK |
| 21 | Kappa-C | RTVAAPSVFIFPPSDEQLKSGTASVVCLLNNFYPREAKVQWKVDN |
| | | ALQSGNSQESVTEQDSKDSTYSLSSTLTLSKADYEKHKVYACEVTH |
| | | QGLSSPVTKSFNRGEC |
| 22 | Lambda-C | GQPKAAPSVTLFPPSSEELQANKATLVCLISDFYPGAVTVAWKAD |
| | | SSPVKAGVETTTPSKQSNNKYAASSYLSLTPEQWKSHRSYSCQVT |
| | | HEGSTVEKTVAPTECS |
| 23 | Human CD137 | MGNSCYNIVALLLVLNFERTRSLQDPCSNCPAGTFCDNNRNQICS |
| | (UniProtKB-Q07011; | PCPPNSFSSAGGQRTCDICRQCKGVFRTRKECSSTSNAECDCTPG |
| | incl. signal peptide | FHCLGAGCSMCEQDCKQGQELTKKGCKDCCFGTFNDQKRGICRP |
| | sequence: aa 1-23) | WTNCSLDGKSVLVNGTKERDVVCGPSPADLSPGASSVTPPAPAR |
| | | EPGHSPQIISFFLALTSTALLFLLFFLTLRFSVVKRGRKKLLYIFKQPF |
| | | MRPVQTTQEEDGCSCRFPEEEEGGCEL |
| 24 | Human CD137 | LQDPCSNCPAGTFCDNNRNQICSPCPPNSFSSAGGQRTCDICRQ |
| | (UniProtKB-Q07011; | CKGVFRTRKECSSTSNAECDCTPGFHCLGAGCSMCEQDCKQGQE |
| | mature sequence) | LTKKGCKDCCFGTFNDQKRGICRPWTNCSLDGKSVLVNGTKERD |
| | | VVCGPSPADLSPGASSVTPPAPAREPGHSPQIISFFLALTSTALLFLL |
| | | FFLTLRFSVVKRGRKKLLYIFKQPFMRPVQTTQEEDGCSCRFPEEEE |
| | | GGCEL |
| 25 | Human PD-L1 | MRIFAVFIFMTYWHLLNAFTVTVPKDLYVVEYGSNMTIECFPVEK |
| | (UniProtKB-Q9NZQ7; | QLDLAALIVYWEMEDKNIIQFVHGEEDLKVQHSSYRQRARLLKDQ |
| | incl. signal peptide | LSLGNAALQITDVKLQDAGVYRCMISYGGADYKRITVKVNAPYNK |
| | sequence: aa 1-18) | INQRILVVDPVTSEHELTCQAEGYPKAEVIWTSSDHQVLSGKTTTT |
| | | NSKREEKLFNVTSTLRINTTTNEIFYCTFRRLDPEENHTAELVIPELPL |
| | | AHPPNERTHLVILGAILLCLGVALTFIFRLRKGRMMDVKKCGIQDT |
| | | NSKKQSDTHLEET |
| 26 | Human PD-L1 | FTVTVPKDLYVVEYGSNMTIECKFPVEKQLDLAALIVYWEMEDKN |
| | (UniProtKB-Q9NZQ7; | IIQFVHGEEDLKVQHSSYRQRARLLKDQLSLGNAALQITDVKLQDA |
| | mature sequence) | GVYRCMISYGGADYKRITVKVNAPYNKINQRILVVDPVTSEHELTC |
| | | QAEGYPKAEVIWTSSDHQVLSGKTTTTNSKREEKLFNVTSTLRINT |
| | | TTNEIFYCTFRRLDPEENHTAELVIPELPLAHPPNERTHLVILGAILLC |
| | | LGVALTFIFRLRKGRMMDVKKCGIQDTNSKKQSDTHLEET |
| 27 | Homo Sapiens EGFR | MRPSGTAGAALLALLAALCPASRALEEKKVCQGTSNKLTQLGTFE |
| | | DHFLSLQRMFNNCEVVLGNLEITYVQRNYDLSFLKTIQEVAGYVLI |
| | | ALNTVERIPLENLQIIRGNMYYENSYALAVLSNYDANKTGLKELPM |
| | | RNLQEILHGAVRFSNNPALCNVESIQWRDIVSSDFLSNMSMDFQ |
| | | NHLGSCQKCDPSCPNGSCWGAGEENCQKLTKIICAQQCSGRCRG |
| | | KSPSDCCHNQCAAGCTGPRESDCLVCRKFRDEATCKDTCPPLMLY |
| | | NPTTYQMDVNPEGKYSFGATCVKKCPRNYVVTDHGSCVRACGA |
| | | DSYEMEEDGVRKCKKCEGPCRKVCNGIGIGEFKDSLSINATNIKHF |
| | | KNCTSISGDLHILPVAFRGDSFTHTPPLDPQELDILKTVKEITGFLLI |
| | | QAWPENRTDLHAFENLEIIRGRTKQHGQFSLAVVSLNITSLGLRSL |
| | | KEISDGDVIISGNKNLCYANTINWKKLFGTSGQKTKIISNRGENSCK |
| | | ATGQVCHALCSPEGCWGPEPRDCVSCRNVSRGRECVDKCNLLEG |
| | | EPREFVENSECIQCHPECLPQAMNITCTGRGPDNCIQCAHYIDGP |
| | | HCVKTCPAGVMGENNTLVWKYADAGHVCHLCHPNCTYGCTGP |
| | | GLEGCPTNGPKIPSIATGMVGALLLLLVVALGIGLFMRRRHIVRKR |
| | | TLRRLLQERELVEPLTPSGEAPNQALLRILKETEFKKIKVLGSGAFGT |
| | | VYKGLWIPEGEKVKIPVAIKELREATSPKANKEILDEAYVMASVDN |
| | | PHVCRLLGICLTSTVQLITQLMPFGCLLDYVREHKDNIGSQYLLNW |
| | | CVQIAKGMNYLEDRRLVHRDLAARNVLVKTPQHVKITDFGLAKLL |
| | | GAEEKEYHAEGGKVPIKWMALESILHRIYTQSDVWSYGVTVWEL |
| | | MTFGSKPYDGIPASEISSILEKGERLPQPPICTIDVYMIMVKCWMI |
| | | DADSRPKFRELIIEFSKMARDPQRYLVIQGDERMHLPSPTDSNFYR |
| | | ALMDEEDMDDVVDADEYLIPQQGFFSSPSTSRTPLLSSLSATSNN |
| | | STVACIDRNGLQSCPIKEDSFLQRYSSDPTGALTEDSIDDTFLPVPE |
| | | YINQSVPKRPAGSVQNPVYHNQPLNPAPSRDPHYQDPHSTAVG |
| | | NPEYLNTVQPTCVNSTFDSPAHWAQKGSHQISLDNPDYQQDFFP |
| | | KEAKPNGIFKGSTAENAEYLRVAPQSSEFIGA |
| 28 | VH_CD137-009 | QSLEESGGRLVTPGTPLTLTCTVSGFSLNDYWMSWVRQAPGKGL |
| | | EWIGYIDVGGSLYYASWAKGRFTISRTSTTVDLKMTSLTTEDTATY |
| | | FCARGGLTYGFDLWGPGTLVTVSS |
| 29 | VL_CD137-009 | DIVMTQTPASVSEPVGGTVTINCQASEDISSYLAWYQQKPGQRP |
| | | KRLIYGASDLASGVPSRFSASGSGTEYALTISDLESADAATYYCHYY |
| | | ATISGLGVAFGGGTEVVVK |
| 30 | IgG1-Fc without C- | ASTKGPSVFPLAPSSKSTSGGTAALGCLVKDYFPEPVTVSWNSGAL |
| | terminal Lysine | TSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFFLYSKLTVDKSRWQQGNVFSCSVMHEALH |
| | | NHYTQKSLSLSPG |
| 31 | IgG1-Fc_F405L without C- | ASTKGPSVFPLAPSSKSTSGGTAALGCLVKDYFPEPVTVSWNSGAL |
| | terminal Lysine | TSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFLLYSKLTVDKSRWQQGNVFSCSVMHEALH |
| | | NHYTQKSLSLSPG |
| 32 | IgG1-Fc_K409R without | ASTKGPSVFPLAPSSKSTSGGTAALGCLVKDYFPEPVTVSWNSGAL |
| | C-terminal Lysine | TSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFFLYSRLTVDKSRWQQGNVFSCSVMHEALH |
| | | NHYTQKSLSLSPG |
| 33 | IgG1-Fc_FEA without C- | ASTKGPSVFPLAPSSKSTSGGTAALGCLVKDYFPEPVTVSWNSGAL |
| | terminal Lysine | TSGVHTFPAVLQSSGLYSLSSVVTVPSSSLGTQTYICNVNHKPSNT |
| | | KVDKRVEPKSCDKTHTCPPCPAPEFEGGPSVFLFPPKPKDTLMISR |
| | | TPEVTCVVVAVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFFLYSKLTVDKSRWQQGNVFSCSVMHEALH |
| | | NHYTQKSLSLSPG |
| 34 | IgG1-FEAR-Fc without C- | ASTKGPSVFPLAPSSKSTSGGTAALGCLVKDYFPEPVTVSWNSGAL |
| | terminal Lysine | TSGVHTFPAVLQSSGLYSLSSVVTVPSSSLGTQTYICNVNHKPSNT |
| | | KVDKRVEPKSCDKTHTCPPCPAPEFEGGPSVFLFPPKPKDTLMISR |
| | | TPEVTCVVVAVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFFLYSRLTVDKSRWQQGNVFSCSVMHEALH |
| | | NHYTQKSLSLSPG |
| 35 | IgG1-FEAL-Fc without C- | ASTKGPSVFPLAPSSKSTSGGTAALGCLVKDYFPEPVTVSWNSGAL |
| | terminal Lysine | TSGVHTFPAVLQSSGLYSLSSVVTVPSSSLGTQTYICNVNHKPSNT |
| | | KVDKRVEPKSCDKTHTCPPCPAPEFEGGPSVFLFPPKPKDTLMISR |
| | | TPEVTCVVVAVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSREEMTKNQVSLTCLVKGFYPSDIAVEWESNGQPEN |
| | | NYKTTPPVLDSDGSFLLYSKLTVDKSRWQQGNVFSCSVMHEALH |
| | | NHYTQKSLSLSPG |
| 36 | CD137-009 heavy chain | EVQLVESGGGLVQPGRSLRLSCTASGFSLNDYWMSWVRQAPGK |
| | | GLEWVGYIDVGGSLYYAASVKGRFTISRDDSKSIAYLQMNSLKTED |
| | | TAVYYCARGGLTYGFDLWGQGTLVTVSSASTKGPSVFPLAPSSKS |
| | | TSGGTAALGCLVKDYFPEPVTVSWNSGALTSGVHTFPAVLQSSGL |
| | | YSLSSVVTVPSSSLGTQTYICNVNHKPSNTKVDKRVEPKSCDKTHT |
| | | CPPCPAPEFEGGPSVFLFPPKPKDTLMISRTPEVTCVVVAVSHEDP |
| | | EVKFNWYVDGVEVHNAKTKPREEQYNSTYRVVSVLTVLHQDWL |
| | | NGKEYKCKVSNKALPAPIEKTISKAKGQPREPQVYTLPPSREEMTK |
| | | NQVSLTCLVKGFYPSDIAVEWESNGQPENNYKTTPPVLDSDGSFF |
| | | LYSRLTVDKSRWQQGNVFSCSVMHEALHNHYTQKS LSLSPG |
| 37 | CD137-009 light chain | DIVMTQSPSSLSASVGDRVTITCQASEDISSYLAWYQQKPGKAPK |
| | | RLIYGASDLASGVPSRFSASGSGTDYTFTISSLQPEDIATYYCHYYAT |
| | | ISGLGVAFGGGTKVEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |
| 38 | PD-L1-547 heavy chain | EVQLLEPGGGLVQPGGSLRLSCEASGSTFSTYAMSWVRQAPGKG |
| | | LEWVSGFSGSGGFTFYADSVRGRFTISRDSSKNTLFLQMSSLRAED |
| | | TAVYYCAIPARGYNYGSFQHWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCDK |
| | | THTCPPCPAPEFEGGPSVFLFPPKPKDTLMISRTPEVTCVVVAVSH |
| | | EDPEVKFNWYVDGVEVHNAKTKPREEQYNSTYRVVSVLTVLHQD |
| | | WLNGKEYKCKVSNKALPAPIEKTISKAKGQPREPQVYTLPPSREE |
| | | MTKNQVSLTCLVKGFYPSDIAVEWESNGQPENNYKTTPPVLDSD |
| | | GSFLLYSKLTVDKSRWQQGNVFSCSVMHEALHNHYTQKSLSLSP |
| | | G |
| 39 | PD-L1-547 light chain | SYVLTQPPSVSVAPGQTARITCGGNNIGSKSVHWYQQKPGQAPV |
| | | LVVYDDNDRPSGLPERFSGSNSGNTATLTISRVEAGDEADYYCQV |
| | | WDSSSDHVVFGGGTKLTVLGQPKAAPSVTLFPPSSEELQANKATL |
| | | VCLISDFYPGAVTVAWKADSSPVKAGVETTTPSKQSNNKYAASSY |
| | | LSLTPEQWKSHRSYSCQVTHEGSTVEKTVAPTECS |

Bold and underlined are F; E; A; L and R, corresponding with positions 234 and 235; 265; 405 and 409, respectively, said positions being in accordance with EU-numbering.
In variable regions, said CDR regions that were annotated in accordance with IMGT definitions, are underlined.

The present invention is further illustrated by the following examples, which should not be construed as limiting the scope of the invention.

### EXAMPLES

### Example 1: Generation of CD137 Antibody

The antibodies CD137-005 and CD137-009 were generated as described in example 1 of WO2016/110584. In short, rabbits were immunized with a mixture of proteins containing a human CD137-Fc fusion protein. Single B cells from blood were sorted and screened for production of CD137 specific antibody by ELISA and flow cytometry. From screening-positive B cells, RNA was extracted and sequencing was performed. The variable regions of heavy and light chain were gene synthesized and cloned into a human IgG1 kappa expression vector or human IgG1 lambda expression vector including a human IgG1 heavy chain containing the following amino acid mutations: L234F, L235E, D265A and F405L (FEAL) or F405L (FEAL) wherein the amino acid position number is according to EU numbering (correspond to SEQ ID NO: 20). The variable region sequences of the chimeric CD137 antibody (CD137-009) are shown in the Sequence Listing SEQ ID NO: 28 and SEQ ID NO: 29 herein.

### Example 2: Humanization of the Rabbit (chimeric) CD137 Antibody

Humanized antibody sequences from the rabbit anti-CD137-009 were generated at Antitope (Cambridge, UK). Humanized antibody sequences were generated using germline humanization (CDR-grafting) technology. Humanized V region genes were designed based on human germline sequences with closest homology to the VH and VK amino acid sequences of the rabbit antibody. A series of seven VH and three VK (VL) germline humanized V-region genes were designed. Structural models of the non-human parental antibody V regions were produced using Swiss PDB and analyzed in order to identify amino acids in the V region frameworks that may be important for the binding properties of the antibody. These amino acids were noted for incorporation into one or more variant CDR-grafted antibodies. The germline sequences used as the basis for the humanized designs are shown in Table 4.

TABLE 4
Closest matching human germline V
segment and J segment sequences.

| | Heavy chain | | Light chain (κ) | |
|---|---|---|---|---|
| | Human V | Human J | Human V | Human J |
| | region | region | region | region |
| | germline | germline | germline | germline |
| Antibody | segment | segment | segment | segment |
| Rabbit anti- | hIGHV3-49*04 | hIGHJ4 | hIGKV1-33*01 | IGKJ4 |
| CD137-009 | | | | |

Variant sequences with the lowest incidence of potential T cell epitopes were then selected using Antitope's proprietary in silico technologies, iTope™ and TCED™ (T Cell Epitope Database) (Perry, L. C. A, Jones, T. D. and Baker, M. P. New Approaches to Prediction of Immune Responses to Therapeutic Proteins during Preclinical Development (2008). Drugs in R&D 9 (6): 385-396; 20 Bryson, C. J., Jones, T. D. and Baker, M. P. Prediction of Immunogenicity of Therapeutic Proteins (2010). Biodrugs 24 (1):1-8). Finally, the nucleotide sequences of the designed variants have been codon-optimized.

The variable region sequences of the humanized CD137 antibody (CD137-009-HC7LC2) are shown in the Sequence Listing SEQ ID NO: 1 and SEQ ID NO: 5 herein.

### Example 3: Generation of PD-L1 Antibody

Immunization and hybridoma generation were performed at Aldevron GmbH (Freiburg, Germany). A cDNA encoding amino acid 19-238 of human PD-L1 was cloned into Aldevron proprietary expression plasmids. Antibody PD-L1-547 was generated by immunization of OmniRat animals (transgenic rats expressing a diversified repertoire of antibodies with fully human idiotypes; Ligand Pharmaceuticals Inc., San Diego, USA) using intradermal application of human PD-L1 cDNA-coated gold-particles using a hand-held device for particle-bombardment (“gene gun”). Serum samples were collected after a series of immunizations and tested in flow cytometry on HEK cells transiently transfected with the aforementioned expression plasmids to express human PD-L1. Antibody-producing cells were isolated and fused with mouse myeloma cells (Ag8) according to standard procedures. RNA from hybridomas producing PD-L1 specific antibody was extracted and sequencing was performed. The variable regions of heavy and light chain (SEQ ID NOs: 8 and 12) were gene synthesized and cloned into a human IgG1 lambda expression vector including a human IgG1 heavy chain containing the following amino acid mutations: L234F, L235E, D265A and K409R (FEAR) wherein the amino acid position number is according to EU numbering (correspond to SEQ ID NO: 19).

### Example 4: Generation of Bispecific Antibodies by 2-MEA-Induced Fab-Arm Exchange

Bispecific IgG1 antibodies were generated by Fab-arm-exchange under controlled reducing conditions. The basis for this method is the use of complementary CH3 domains, which promote the formation of heterodimers under specific assay conditions as described in WO2011/131746. The F405L and K409R (EU numbering) mutations were introduced into the relevant antibodies to create antibody pairs with complementary CH3 domains.

To generate bispecific antibodies, the two parental complementary antibodies, each antibody at a final concentration of 0.5 mg/ml, were incubated with 75 mM 2-mercaptoethylamine-HCl (2-MEA) in a total volume of 100 μL PBS at 31° C. for 5 hours. The reduction reaction was stopped by removing the reducing agent 2-MEA using spin columns (Microcon centrifugal filters, 30 k, Millipore) according to the manufacturer's protocol.

Bispecific antibodies were generated by combining the following antibodies from Example 1 and 4:
CD137-009-FEAL antibody combined with the PD-L1-547-FEAR antibody
PD-L1-547-FEAL antibody combined with the CD137-009-FEAR antibody
GEN1046 (PD-L1-547-FEAL antibody combined with CD137-009-HC7LC2-FEAR antibody),
b12-FEAL antibody combined with the PD-L1-547-FEAR antibody, with CD137-009-FEAR or with CD137-009-HC7LC2-FEAR antibody, using as the first arm the antibody b12 which is a gp120 specific antibody (Barbas, C F. J Mol Biol. 1993 Apr 5;230(3):812-23)
PD-L1-547-FEAL or CD137-009-FEAL with b12-FEAR antibody.

The following sequences were used for the heavy and light chains, respectively:

PD-L1-547-FEAL
VH-PD-L1-547 (SEQ ID NO: 8),
IgG1-FEAL-Fc (SEQ ID NO: 20)
VL-PD-L1-547 (SEQ ID NO: 12),
Lambda-C (SEQ ID NO: 22)
PD-L1-547-FEAR
VH-PD-L1-547 (SEQ ID NO: 8),
IgG1-FEAR-Fc (SEQ ID NO: 19)
VL-PD-L1-547 (SEQ ID NO: 12),
Lambda-C (SEQ ID NO: 22)
CD137-009-FEAL:
VH_CD137-009 (SEQ ID NO 28),
IgG1-FEAL-Fc (SEQ ID NO: 20)
VL_CD137-009 (SEQ ID NO 29),
Kappa-C (SEQ ID NO: 21)
CD137-009-HC7LC2-FEAR
VH_CD137-009-H7 (SEQ ID NO: 1),
IgG1-FEAR-Fc (SEQ ID NO: 19)
VL_CD137-009-L2 (SEQ ID NO: 5),
Kappa-C (SEQ ID NO: 21)
CD137-009-FEAR:
VH_CD137-009 (SEQ ID NO 28),
IgG1-FEAR-Fc (SEQ ID NO: 19)
VL_CD137-009 (SEQ ID NO 29),
Kappa-C (SEQ ID NO: 21)

### Example 5: Simultaneous Binding of GEN1046 to PD-L1 and CD137-Expressing Cells

To measure the dose-response of simultaneous binding of GEN1046 to human PD-L1- and CD137-expressing cells, transgenic K562 cells were differently labelled with fluorescent dyes and the formation of doublets analyzed by flow cytometry.

K562 cells transgenic for human PD-L1 (K562_hPD-L1; 6×10⁶ cells) were fluorescently labelled with the CellTrace™ Violet Cell Proliferation Kit (Cat. no. C34557, Thermo Fisher Scientific GmbH, Dreieich, Germany) in 2 mL of a 2.5 μM staining solution for 10 minutes at 37° C. In parallel, K562 cells transgenic for human CD137 (K562_h4-1BB; 6×10⁶ cells) were fluorescently labelled with the CellTrace™ Far Red Cell Proliferation Kit (Cat. no. C34564, Thermo Fisher Scientific GmbH, Dreieich, Germany) in 2 mL of a 0.5 μM staining solution for 10 minutes at 37° C. Staining was stopped by adding 4 mL fetale bovine serum (FBS; Cat. no. S0115, Biochrom GmbH, Berlin, Germany). After washing once in RPMI1640 (Cat. no. 11875093, Thermo Fisher Scientific GmbH, Dreieich, Germany) supplemented with 10% FBS, stained K56_hPD-L1 and K562_h4-1BB cells were combined at a ratio of 1:1 and adjusted to 1.25×10⁶ cells/mL in RPMI1640, 10% FBS. Combined K562_hPD-L1 and K562_h4-1BB cells were transferred into polystyrene 5 mL round-bottom tubes (Cat No. 10579511, Fisher Scientific, Schwerte, Germany) (1×10⁶ cells/tube). Cells were incubated with serial dilutions of antibodies (range 0.001 to 100 μg/mL in 10-fold dilution steps) in RPMI1640, 10% FBS at 37° C. for 15 minutes. Samples were immediately analyzed on a FACS Canto™ II flow cytometer (Becton Dickinson GmbH, Heidelberg, Germany) without prior mixing in order to preserve formed doublets. K562_hPD-L1/K562_h4-1BB doublets were identified as CellTrace™ Violet/CellTrace™ Far Red double-positive population by FlowJo 10.4 software. The percent double-positive cells was plotted as a function of antibody concentration using GraphPad Prism version 8.01 (GraphPad Software, Inc).

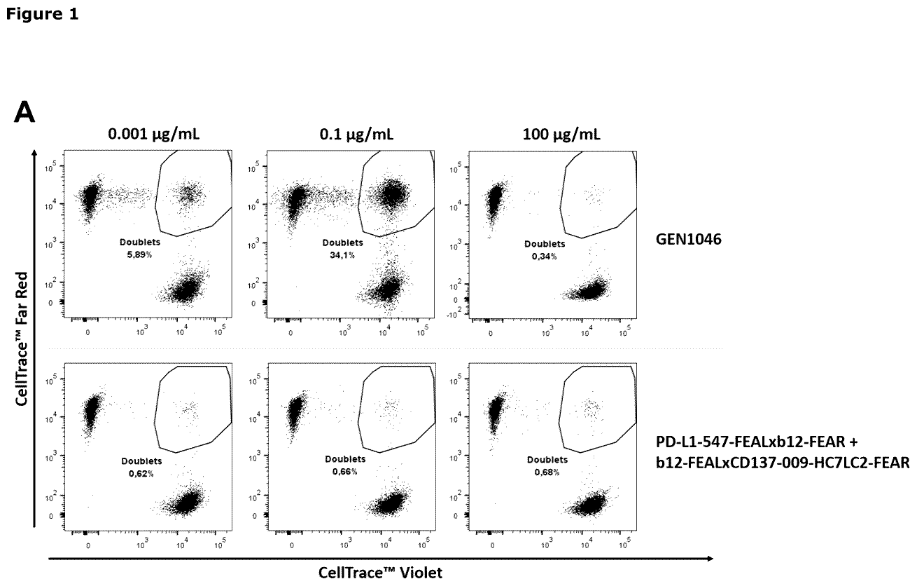

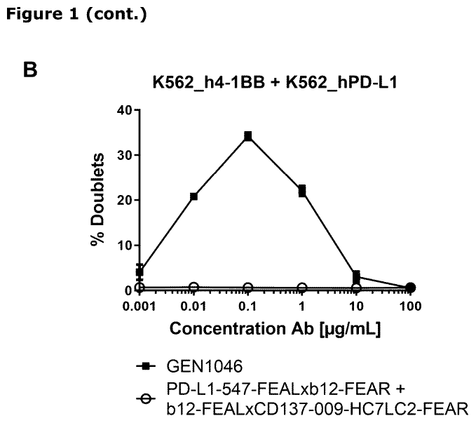

FIG. 1A shows that the addition of GEN1046 induced the formation of CellTrace™ Violet/CellTrace™ Far Red double-positive doublets. K562_hPD-L1/K562_h4-1BB co-cultures incubated with an intermediate GEN1046 concentration of 0.1 μg/mL displayed the most prominent doublet formation, whereas only moderate doublet formation was observed at a low GEN1046 concentration of 0.001 μg/mL and a minimal to absent doublet formation was detectable at a high GEN1046 concentration of 100 μg/mL. This observation is in-line with the bell-shaped dose response curve displayed in FIG. 1B covering the tested antibody concentration range of 0.001 μg/mL to 100 μg/mL. In contrast to GEN1046, the combination of monovalent PD-L1 and CD137 control antibodies, PD-L1-547-FEALxb12-FEAR and b12-FEALxCD137-009-HC7LC2-FEAR, resulted in no doublet formation at all antibody concentrations tested.

### Example 6: Effect of GEN1046 in CD137 Reporter Assay

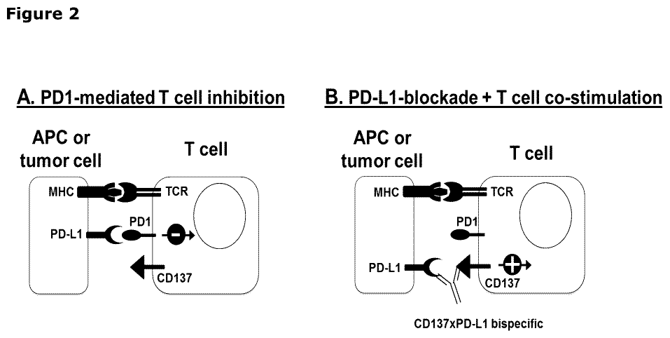

A schematic representation of the anticipated mode of action of PD-L1xCD137 bispecific antibodies is shown in FIG. 2.

To determine the dose-response of GEN1046 to mediate PD-L1-binding dependent CD137 agonist activity, a luciferase based CD137 activation reporter assay was performed with adherent growing human tumor cell lines as PD-L1 source.

Endogenously PD-L1-expressing human ES-2 (ovarian clear cell carcinoma; ATCC® CRL-1978™) and MDA-MB-231 (breast adenocarcinoma; ATCC® HTB-26™) cells were seeded in white flat-bottom 96-well plates (Cat. No. 136101, Thermo Fisher Scientific GmbH, Dreieich, Germany) at a density of 3×10⁴ cells/well in DMEM (Cat. No. 10566016, Thermo Fisher Scientific GmbH, Dreieich, Germany) and incubated overnight at 37° C. Cryo-conserved Thaw-and-use GloResponse™ NFkB-Luc2P/4-1BB Jurkat reporter cells (Cat. No. CS196003, Promega GmbH, Walldorf, Germany) were thawed the next day and the contents of a single vial transferred to a 15 ml tube containing 9.5 mL prewarmed RPMI-1640 supplemented with 1% FBS. Culture medium of the adherent ES-2 and MDA-MB-231 cells was discarded and the co-culture initiated by seeding 50 μL NFkB-Luc2P/4-1BB Jurkat cell suspension on top of the ES-2 or MDA-MB-231 cell monolayer. Cells were incubated with serial dilutions of antibodies (in-assay concentration range 0.00128 to 100 μg/mL in 5-fold dilution steps) in RPMI 1640, 10% FBS at 37° C. for 6 hours. Next, the assay plate was removed from the incubator and equilibrated to room-temperature (RT) for 10 minutes. Bio-Glo™ luciferase reagent (Cat. No. G7941, Promega GmbH, Walldorf, Germany) was reconstituted and prewarmed to RT. 75 μL of the luciferase reagent was added per well and incubated for 10 minutes at RT in the dark. The induced luminescence was measured using an Infinite F200 Pro plate reader (Tecan Deutschland GmbH, Crailsheim, Germany).

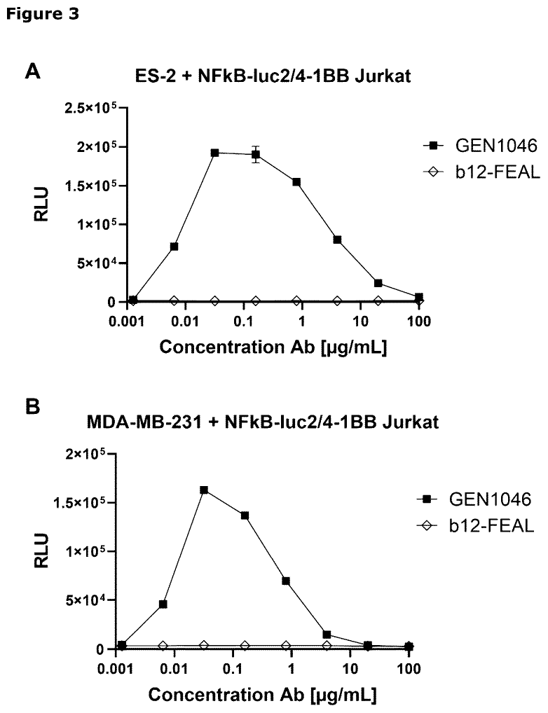

Upon addition of GEN1046 to ES-2: Jurkat (FIG. 3A) and MDA-MB-231: Jurkat reporter cell co-cultures (FIG. 3B), luciferase expression as a read-out for CD137 agonist activation was effectively induced in a concentration-dependent manner following a bell-shaped dose response curve. Whereas an intermediate dose level of around 0.1 μg/ml GEN1046 resulted in the most prominent luminescence signals, lower dose levels as well as higher dose levels were less effective in induction of luciferase expression. Importantly, at very low (0.00128 μg/mL GEN1046) and very high GEN1046 concentrations (100 μg/mL GEN1046), no luciferase expression was detectable. For both co-cultures analyzed, incubation with the b12-FEAL control antibody led to no luciferase expression.

### Example 7: Polyclonal T-Cell Proliferation Assay to Measure Effects of Bispecific Antibodies Binding to PD-L1 and CD137

To measure induction of T-cell proliferation in polyclonally activated T cells, PBMCs were incubated with a sub-optimal concentration of anti-CD3 antibody (clone UCHT1), to activate T cells, combined with bispecific antibody GEN1046 or control antibodies. Within the PBMC population, cells expressing PD-L1 can be bound by the PD-L1-specific arm of the bispecific antibody, whereas activated T cells in the population can be bound by the CD137-specific arm. In this assay, trans-activation of the T cells via the CD137-specific arm, induced by cross-linking with the PD-L1-expressing cells via the bispecific antibody and by blockade of PD-L1:PD-1 interaction, is measured as T-cell proliferation.

PBMCs were obtained from the buffy coat of a healthy donor (Sanquin, Amsterdam, The Netherlands) using a Ficoll gradient (Lonza, lymphocyte separation medium, cat. no. 17-829E). PBMCs were labeled using 0.5 μM carboxyfluorescein succinimidyl ester (CFSE) (Life technologies, cat. no. C34554) in PBS, according to the manufacturer's instructions. 75,000 CFSE-labeled PBMCs were seeded per well in a 96-well round-bottom plate (Greiner bio-one, cat. no. 650180) and incubated with a sub-optimal concentration of anti-CD3 antibody (Stemcell, clone UCHT1, cat. no. 60011; 0.03 μg/mL final concentration) that was pre-determined to induce sub-optimal T cell proliferation, and bispecific or control antibodies (0.0032-10 μg/mL), in 200 μL IMDM GlutaMAX supplemented with 5% human AB serum and 1% penecilin/streptomycin, at 37° C., 5% CO₂, for four days.

Proliferation of different T-cell subsets was analyzed by flow cytometry. Cells were washed in PBS and stained to exclude dead cells with Fixable Viability Stain 510 (50 μL/well; BD Biosciences, cat. no. 564406) at 4° C. for 20 min. After another wash in FACS buffer, cells were stained to distinguish various cellular subsets with a PE-CF594-conjugated CD56-specific antibody (BD BioSciences, cat. no. 564849), a Pacific Blue-conjugated CD4-specific antibody (BioLegend, cat. no. 300521), a AF700-conjugated CD8-specific antibody (BioLegend, cat. no. 301028), a BV711-conjugated CD197-specific antibody (CCR7; BioLegend, cat. no. 353228), a PE-Cy7-conjugated CD45RO-specific antibody (BioLegend, cat. no. 304230), a APC-conjugated CD274-specific antibody (PD-L1; BioLegend cat. no. 329708) and a BV605-conjugated CD137-specific antibody (BioLegend, cat. no. 309822) in FACS buffer at 4° C. for 30 min. Cells were washed three times in FACS buffer and subsequently measured on a FACS Fortessa (BD Biosciences) in 80 μL FACS buffer. CFSE dilution was measured in total T cells and in different T cell subsets (e.g. CCR7⁺CD45RO⁺ central memory T cells and CCR7⁻CD45RO⁺ effector memory T cells). Detailed analyses of T-cell proliferation based on CFSE-peaks indicating cell divisions were made by FlowJo 10.4 software and exported expansion index values were used to plot dose-response curves in GraphPad Prism version 6.04 (GraphPad Software, Inc). The expansion index determines the fold-expansion of the overall culture; an expansion index of 2.0 represents a doubling of the cell count, whereas an expansion index of 1.0 represents no change of the overall cell count.

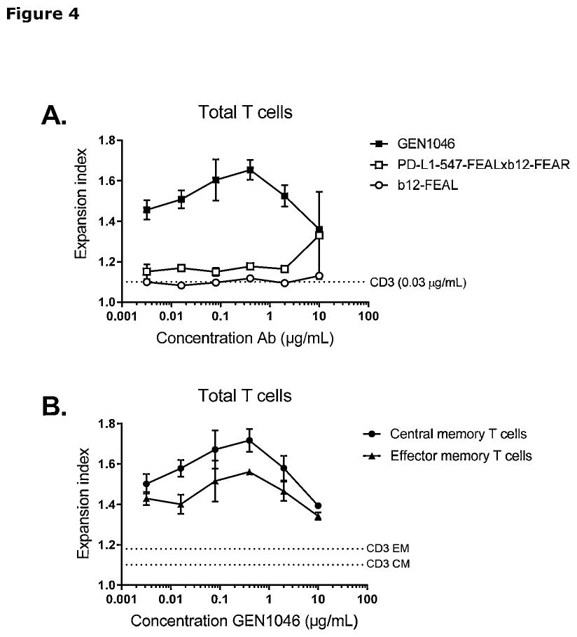

FIG. 4A shows that the bispecific antibody GEN1046 induced expansion of T cells, which was increased compared to CD3 pre-stimulation alone, isotype control antibody b12-FEAL and a monovalent PD-L1-control antibody, PD-L1-547-FEALxb12-FEAR, having one irrelevant arm and one corresponding to the parental bivalent antibody PD-L1-547-FEAR. GEN1046-induced T-cell proliferation was most optimal at 0.4 μg/mL, while at lower and higher concentrations the GEN1046-induced T-cell expansion was less pronounced. When CCR7⁺CD45RO⁺ central memory T cells and CCR7⁻CD45RO⁺ effector memory T cells were analyzed separately (FIG. 4B), a similar pattern emerged, where GEN1046 enhanced T-cell proliferation, which was optimal at 0.4 μg/mL.

### Example 8: Antigen-Specific CD8⁺ T Cell Proliferation Assay to Measure Effects by Bispecific Antibodies Binding to PD-L1 and CD137

To measure induction of T cell proliferation by the bispecific antibody targeting PD-L1 and CD137 in an antigen-specific assay, dendritic cells (DCs) were transfected with claudin-6 in vitro-transcribed RNA (IVT-RNA) to express the claudin-6 antigen. T cells were transfected with PD-1 IVT-RNA and with the claudin-6-specific, HLA-A2-restricted T cell receptor (TCR). This TCR can recognize the claudin-6-derived epitope presented in HLA-A2 on the DC. The PD-L1xCD137 bispecific antibody GEN1046 can cross-link PD-L1 endogenously expressed on monocyte-derived dendritic cells or on tumor cells and CD137 on the T cells, leading to inhibition of the inhibitory PD-1/PD-L1 interaction and at the same time clustering of CD137, resulting in T cell proliferation. Clustering of the CD137 receptor expressed on T cells leads to activation of the CD137 receptor which thereby delivers a co-stimulatory signal to the T cell.

HLA-A2⁺ peripheral blood mononuclear cells (PBMCs) were obtained from healthy donors (Transfusionszentrale, University Hospital, Mainz, Germany). Monocytes were isolated from PBMCs by magnetic-activated cell sorting (MACS) technology using anti-CD14 MicroBeads (Miltenyi; cat. no. 130-050-201), according to the manufacturer's instructions. The peripheral blood lymphocytes (PBLs, CD14-negative fraction) were frozen for future T-cell isolation. For differentiation into immature DCs (IDCs), 1×10⁶ monocytes/ml were cultured for five days in RPMI GlutaMAX (Life technologies GmbH, cat. no. 61870-044) containing 5% human AB serum (Sigma-Aldrich Chemie GmbH, cat. no. H4522-100ML), sodium pyruvate (Life technologies GmbH, cat. no. 11360-039), non-essential amino acids (Life technologies GmbH, cat. no. 11140-035), 100 IU/mL penicillin-streptomycin (Life technologies GmbH, cat. no.15140-122), 1000 IU/mL granulocyte-macrophage colony-stimulating factor (GM-CSF; Miltenyi, cat. no. 130-093-868) and 1,000 IU/mL interleukin-4 (IL-4; Miltenyi, cat. no. 130-093-924). Once during these five days, half of the medium was replaced with fresh medium. iDCs were harvested by collecting non-adherent cells and adherent cells were detached by incubation with PBS containing 2 mM EDTA for 10 min at 37°. After washing, iDCs were frozen in RPMI GlutaMAX containing 10% v/v DMSO (AppliChem GmbH, cat. no A3672,0050)+50% v/v human AB serum for future antigen-specific T cell assays.

One day prior to the start of an antigen-specific CD8⁺ T cell proliferation assay, frozen PBLs and iDCs from the same donor were thawed. CD8⁺ T cells were isolated from PBLs by MACS technology using anti-CD8 MicroBeads (Miltenyi, cat. no. 130-045-201), according to the manufacturer's instructions. About 10-15×10⁶ CD8⁺ T cells were electroporated with 10 μg of in vitro translated (IVT)-RNA encoding the alpha-chain plus 10 μg of IVT-RNA encoding the beta-chain of a claudin-6-specific murine TCR (HLA-A2-restricted; described in WO 2015150327 A1) plus 0.4-10 μg IVT-RNA encoding PD-1 in 250 μL X-Vivo15 (Biozym Scientific GmbH, cat. no.881026) in a 4-mm electroporation cuvette (VWR International GmbH, cat. no. 732-0023) using the BTX ECM® 830 Electroporation System device (BTX; 500 V, 1×3 ms pulse). Immediately after electroporation, cells were transferred into fresh IMDM medium (Life Technologies GmbH, cat. no. 12440-061) supplemented with 5% human AB serum and rested at 37° C., 5% CO₂ for at least 1 hour. T cells were labeled using 1.6 μM carboxyfluorescein succinimidyl ester (CFSE; Invitrogen, cat. no. C34564) in PBS according to the manufacturer's instructions, and incubated in IMDM medium supplemented with 5% human AB serum, O/N.

Up to 5×10⁶ thawed iDCs were electroporated with 0.3-1 μg IVT-RNA encoding full length claudin-6, in 250 μL X-Vivo15 medium, using the electroporation system as described above (300 V, 1×12 ms pulse) and incubated in IMDM medium supplemented with 5% human AB serum, O/N.

The next day, cells were harvested. Cell surface expression of claudin-6 and PD-L1 on DCs and TCR and PD-1 on T cells was checked by flow cytometry. DCs were stained with an Alexa647-conjugated CLDN6-specific antibody (non-commercially available; in-house production) and with anti-human CD274 antibody (PD-L1, eBioscienes, cat. no. 12-5983) and T cells were stained with an anti-Mouse TCR B Chain antibody (Becton Dickinson GmbH, cat. no. 553174) and with anti-human CD279 antibody (PD-1, eBioscienes, cat. no. 17-2799). 5,000 electroporated DCs were incubated with 50,000 electroporated, CFSE-labeled T cells in the presence of bispecific or control antibodies in IMDM GlutaMAX supplemented with 5% human AB serum in a 96-well round-bottom plate. T cell proliferation was measured after 5 days by flow cytometry. Detailed analyses of T-cell proliferation based on CFSE-peaks indicating cell divisions were made by FlowJo 10.4 software and exported expansion index values were used to plot dose-response curves in GraphPad Prism version 6.04 (GraphPad Software, Inc). The expansion index determines the fold-expansion of the overall culture; an expansion index of 2.0 represents a doubling of the cell count, whereas an expansion index of 1.0 represents no change of the overall cell count.

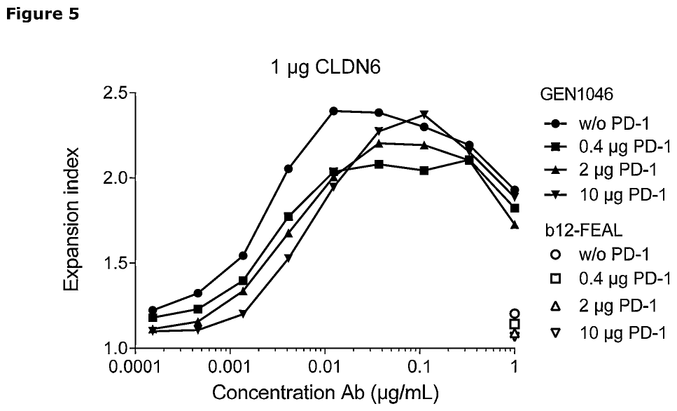

FIG. 5 shows that GEN1046 dose-dependently enhanced T-cell proliferation compared to isotype control antibody b12-FEAL, reflected by an increase in expansion index at concentrations of ≥0.004 μg/mL. GEN1046-induced T-cell proliferation was most optimal at 0.03-0.11 μg/mL, and slightly decreased at the highest concentrations tested, indicative of a bell-shaped dose response curve.

### Example 9: Antigen-Specific CD8⁺ T-Cell Proliferation Assay to Measure Cytokine Release Induced by Bispecific Antibodies Binding to PD-L1 and CD137

The induction of cytokine release by bispecific antibody GEN1046 targeting PD-L1 and CD137 was measured in an antigen-specific assay, performed essentially as described in Example 8.

T cells were electroporated with 10 μg TCR α chain- and 10 μg β chain-encoding RNA, with or without 2 μg PD-1-encoding IVT RNA. Electroporated T cells were not CFSE-labeled (as described supra), but transferred into fresh IMDM medium (Life Technologies GmbH, cat. no. 12440-061) supplemented with 5% human AB serum, immediately after electroporation. iDCs were electroporated with 5 μg claudin-6 (CLDN6)-encoding RNA, as described supra. After O/N incubation, DCs were stained with Alexa647-conjugated CLDN6-specific antibody and T cells with anti-mouse TCR β chain antibody and with anti-human CD279 antibody, as described supra.

5,000 electroporated DCs were incubated with 50,000 electroporated T cells in the presence of different concentrations of bispecific antibody GEN1046 or control antibody b12-FEAL in IMDM GlutaMAX supplemented with 5% human AB serum in a 96-well round-bottom plate. Following a 48-hour incubation period, the plates were centrifuged at 500×g for 5 min and the supernatant was carefully transferred from each well to a fresh 96-well round bottom plate and stored at −80° C. until cytokine analysis on the MSDR platform. The collected supernatants from the antigen-specific proliferation assay were analyzed for cytokine levels of 10 different cytokines by an MSD V-Plex Human Proinflammatory panel 1 (10-Plex) kit (Meso Scale Diagnostics, LLC., cat. no. K15049D-2) on a MESO QuickPlex SQ 120 instrument (Meso Scale Diagnostics, LLC., cat. no. R31QQ-3), according to the manufacturer's instructions.

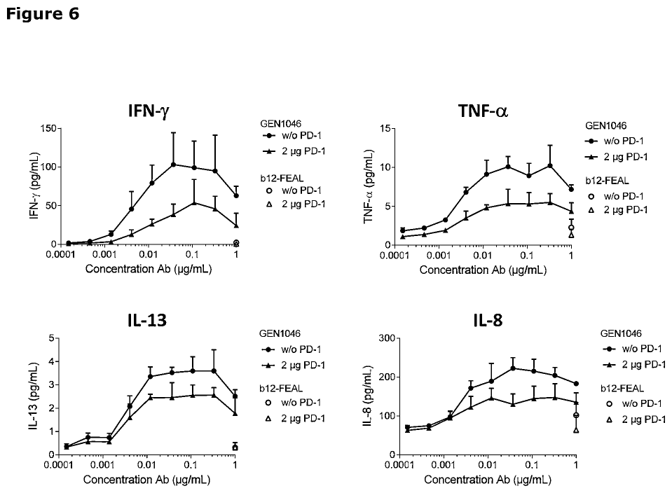

The addition of GEN1046 led to a dose-dependent increase in secretion of primarily IFN-γ, TNF-α, IL-13 and IL-8 (FIG. 6), which was most optimal at concentrations of 0.04-0.33 μg/mL. Lower dose levels as well as a higher dose level of 1 μg/mL were less effective in inducing these cytokines, indicative of a bell-shaped dose response curve. When comparing T cell:DC co-cultures where T cells were not electroporated with PD-1 RNA to those where T cells were electroporated with 2 μg PD-1 RNA, slightly higher cytokine levels were detectable for co-cultures without PD-1 RNA electroporation. This was observed for both the GEN1046 dose response curve as well as for the b12-FEAL control antibody values.

### Example 10: Ex Vivo TIL Expansion Assay to Evaluate the Effects of the CD137xPD-L1 Bispecific Antibody on Tumor Infiltrating Lymphocytes

To evaluate the effects of CD137-009-FEALxPD-L1-547-FEAR on tumor infiltrating lymphocytes (TIL), an ex vivo culture of human tumor tissue was performed as follows. Fresh human tumor tissue resection specimens were washed three times by transferring the isolated tumor chunks from one well in a 6-well plate (Fisher Scientific cat. no. 10110151) containing wash medium to the next using a spatula or serological pipette. Wash medium was composed of X-VIVO 15 (Biozym, cat. no. 881024) supplemented with 1% Pen/Strep (Thermo Fisher, cat. no. 15140-122) and 1% Fungizone (Thermo Fisher, cat. no. 15290-026). Next, the tumor was dissected with a surgical knife (Braun/Roth, cat. no. 5518091 BA223) and cut into pieces with a diameter of about 1-2 mm. Two pieces each were put into one well of a 24-well plate (VWR international, cat. no. 701605) containing 1 mL TIL medium (X-VIVO 15, 10% Human Serum Albumin (HSA, CSL Behring, cat. no. PZN-6446518) 1% Pen/Strep, 1% Fungizone and supplemented with 10 U/mL IL-2 (Proleukin®S, Novartis Pharma, cat. no. 02238131)). CD137-009-FEALxPD-L1-547-FEAR was added at the indicated final concentrations. Culture plates were incubated at 37° C. and 5% CO₂. After 72 hours, 1 mL of fresh TIL medium containing the indicated concentration of the bispecific antibody was added to each well. Wells were monitored via a microscope for the occurrence of TIL clusters every other day. Wells were transferred individually when more than 25 TIL microclusters were detected in the respective well. To split TIL cultures, the cells in the wells of a 24-well plate were re-suspended in the 2 mL medium and transferred into a well of a 6-well plate. Each well was in addition supplemented with another 2 mL of TIL medium.

After a total culture period of 10-14 days, TILs were harvested and analyzed by flow cytometry. Cells were stained with the following reagents, all diluted 1:50 in staining-buffer, (D-PBS containing 5% FCS and 5 mM EDTA), anti-human CD4-FITC (Miltenyi Biotec, cat. no. 130-080-501), anti-human CD3-PE-Cy7 (BD Pharmingen, cat. no. 563423), 7-aminoactinomycin D (7-AAD, Beckman Coulter, cat. no. A07704), anti-human CD56-APC (eBioscience, cat. no. 17-0567-42), and anti-human CD8-PE (TONBO, cat. 50-0088). To allow for quantitative comparison of the acquired cells between different treatment groups, cell pellets were re-suspended after the last washing step in FACS-buffer supplemented with BD™ CompBeads (BD biosciences, cat. no. 51-90-9001291). Flow cytometric analysis was performed on a BD FACSCanto™ II flow cytometer (Becton Dickinson) and acquired data was analyzed using FlowJo 7.6.5 software. The relative viable TIL count, CD3⁺CD8⁺ T cell count, CD3⁺CD4⁺ T cell count and CD3⁻CD56⁺ NK cell count per 1,000 beads correlating to the corresponding well in a 6-well plate was calculated by normalization of the acquired 7AAD-negative cell fraction to the acquired bead counts.

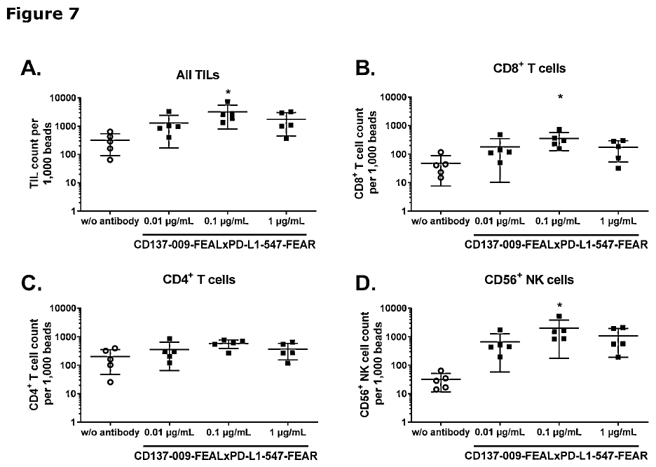

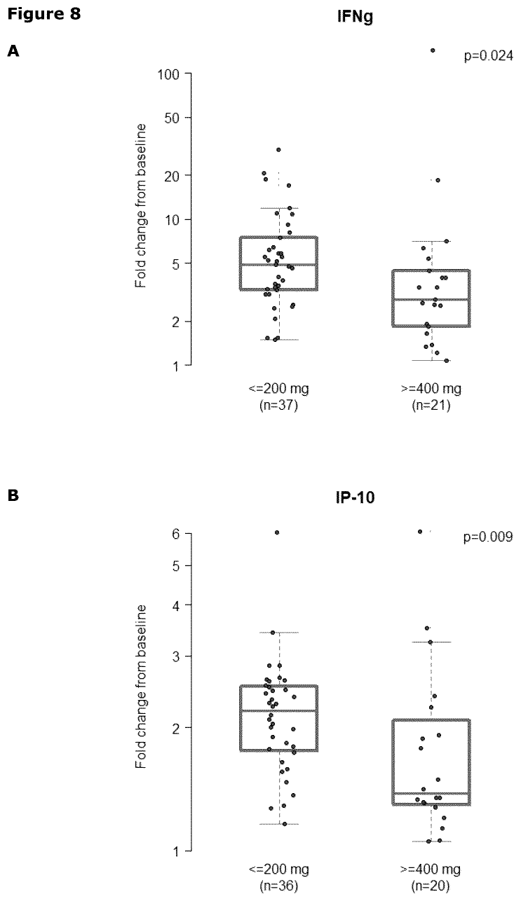

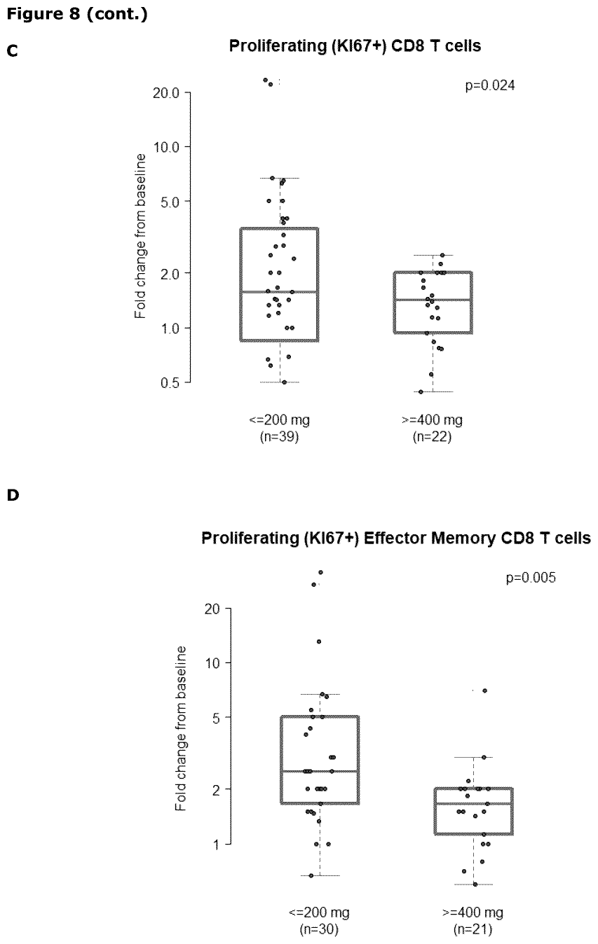

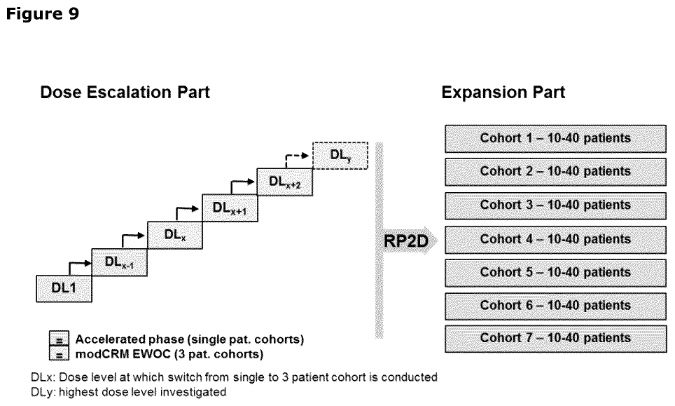

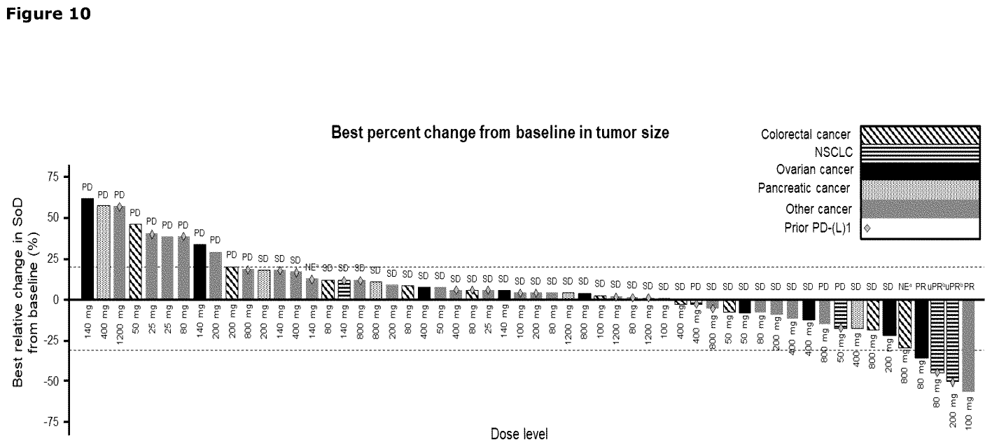

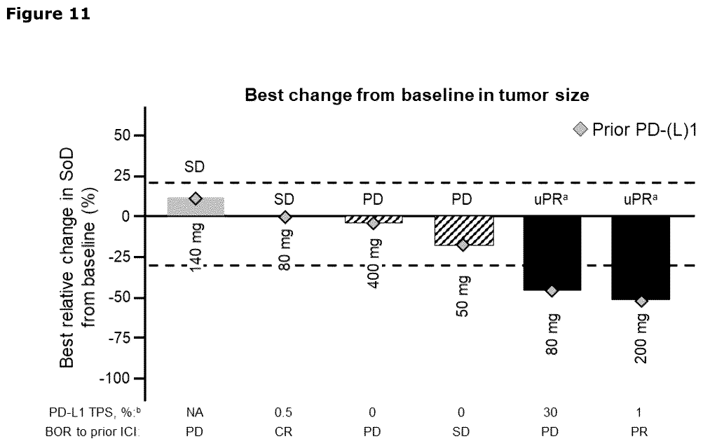

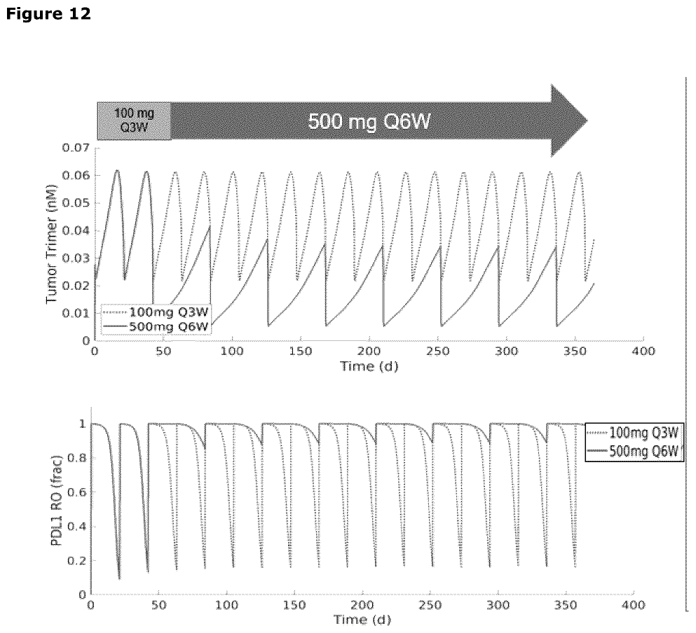

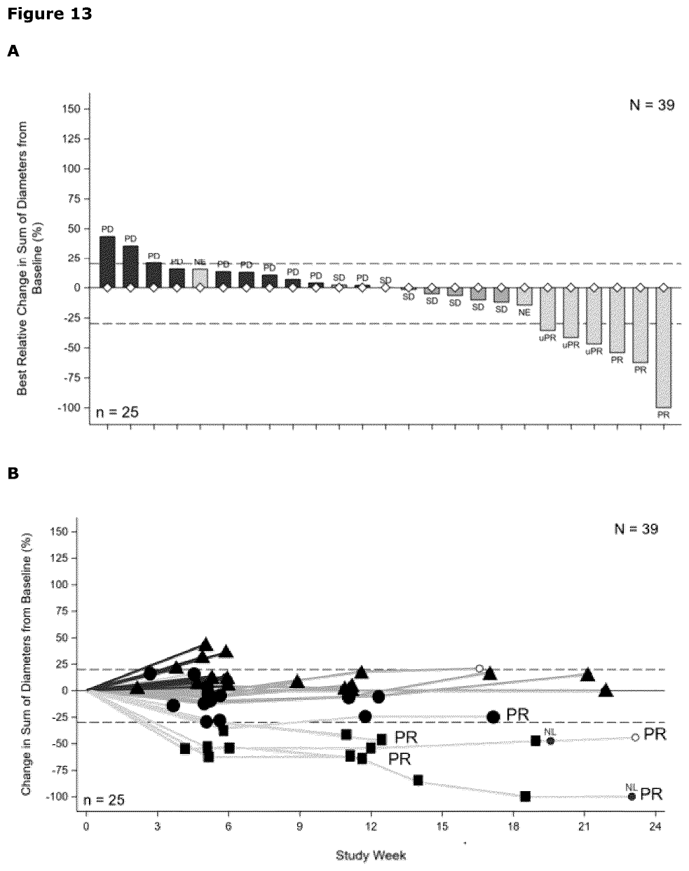

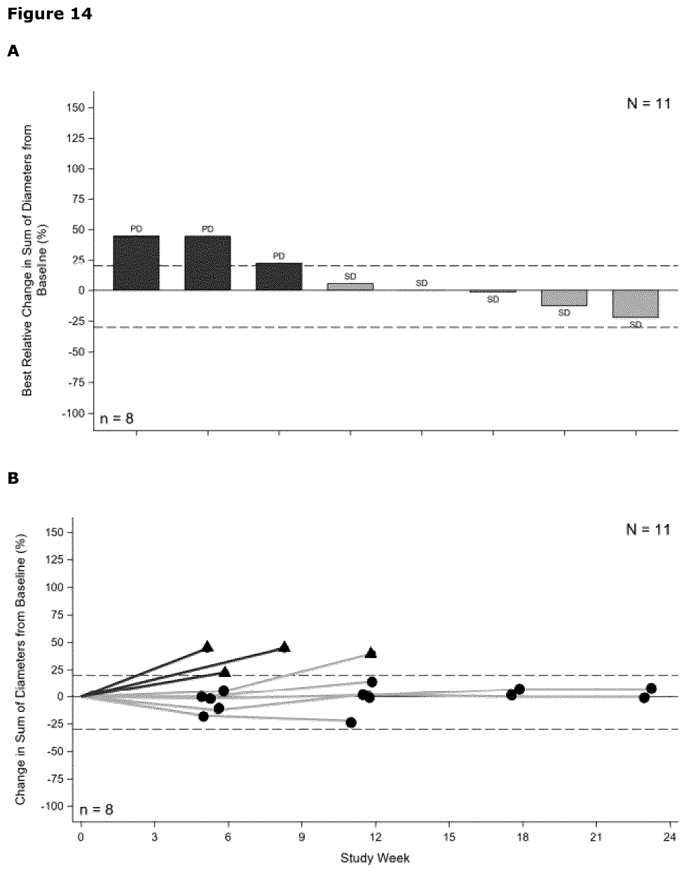

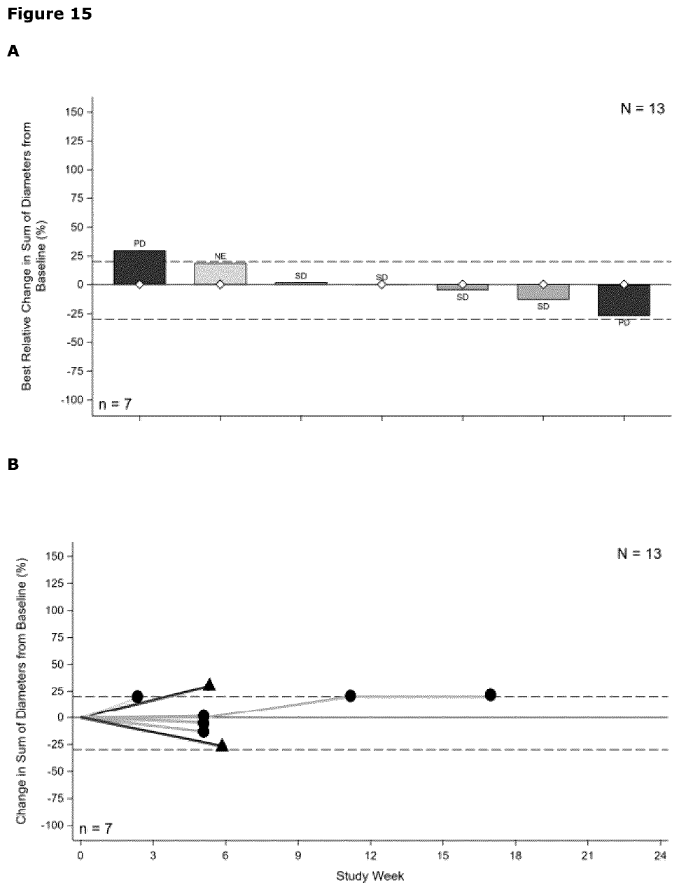

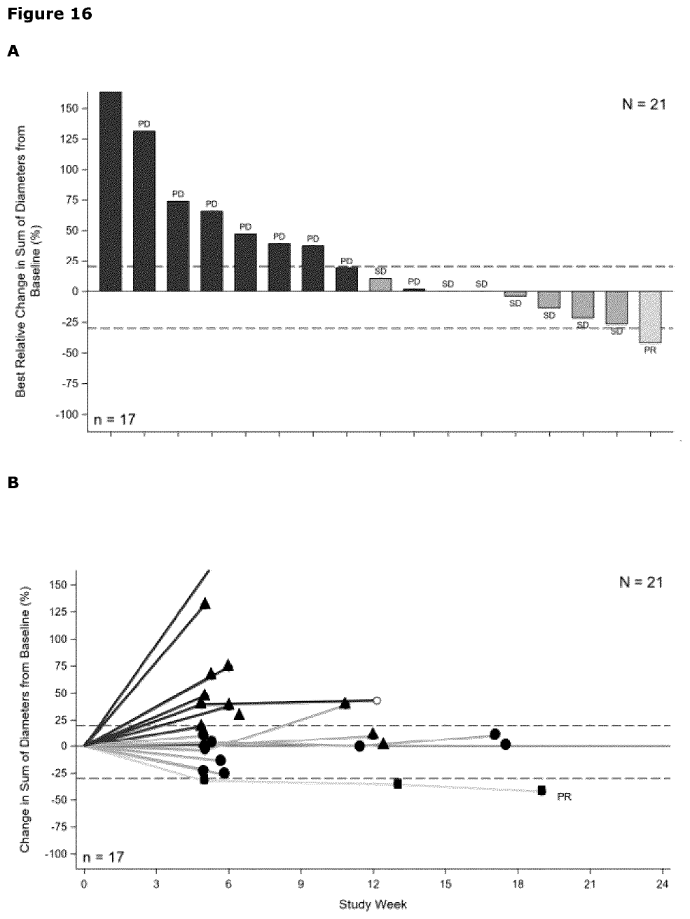

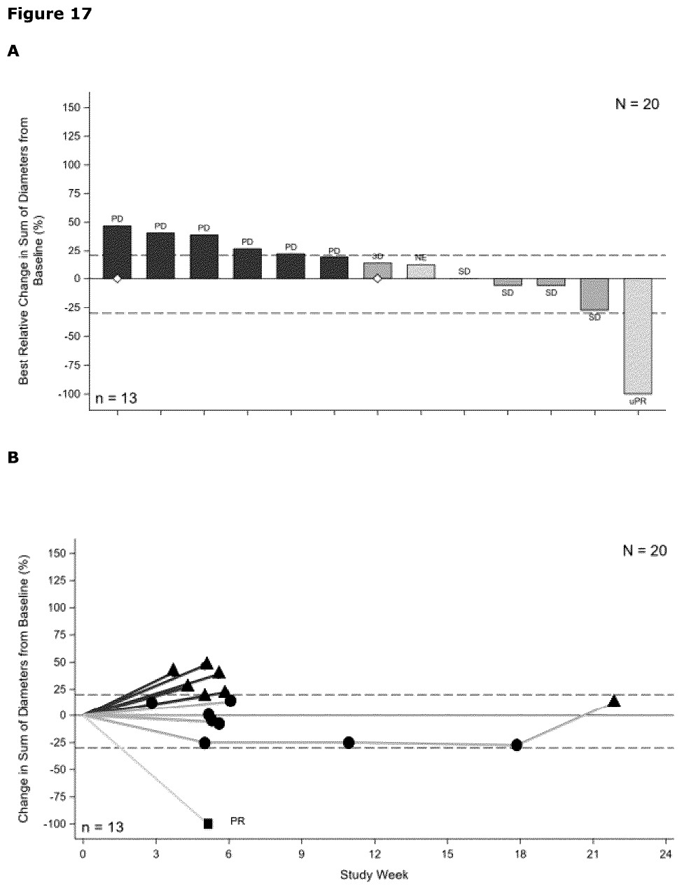

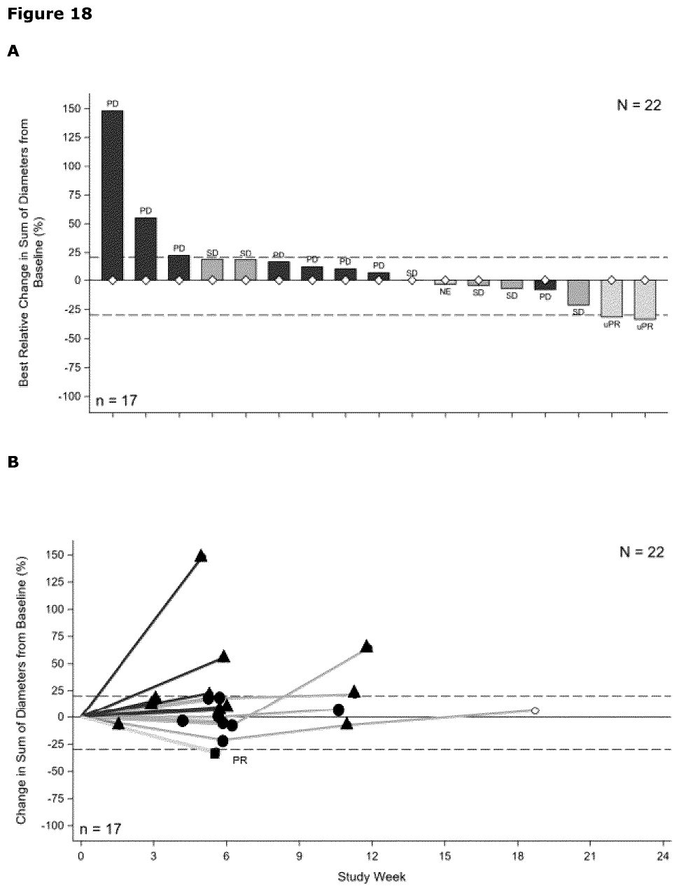

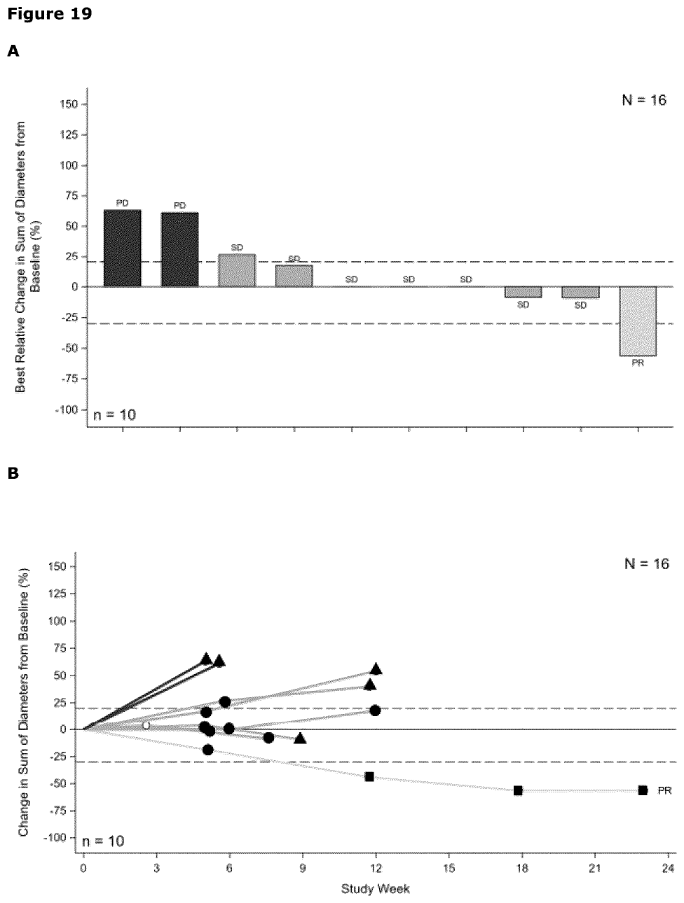

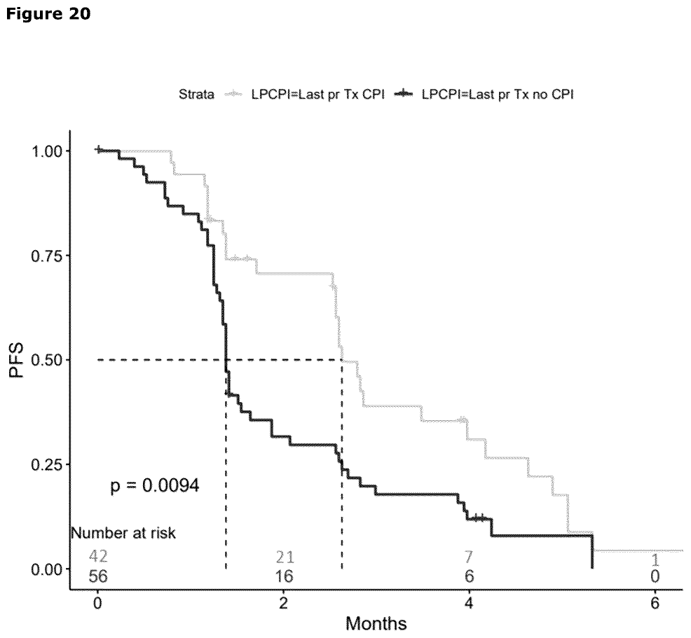

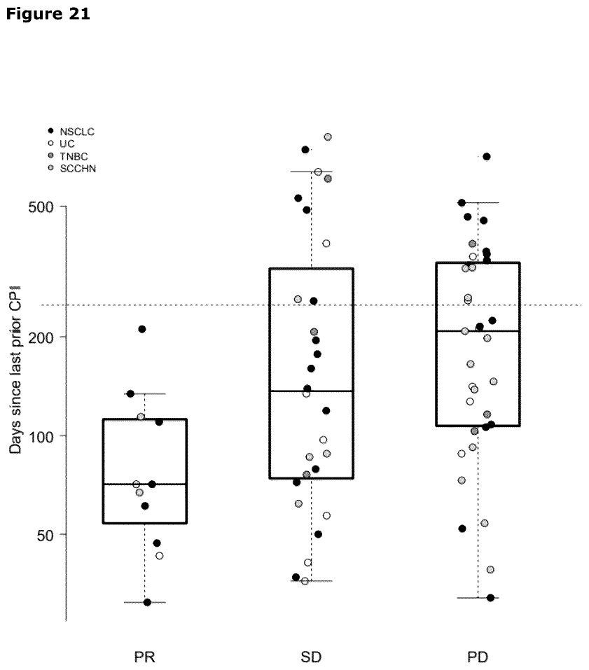

FIG. 7 shows the analysis of a TIL expansion from a human non-small-cell lung carcinoma tissue specimen. Here, the following concentrations of CD137-009-FEALxPD-L1-547-FEAR were added: 0.01, 0.1 and 1 μg/mL; a tissue specimen from the same patient without antibody addition served as negative control. After 10 days of culture, the TILs were harvested and analyzed by flow cytometry. Five samples (from 5 original wells) for each antibody concentration derived from different wells of the 24-well plate were measured. In all samples cultured with the bispecific antibody the viable count of TILs was increased in comparison to the without antibody control samples. Overall, a significant (up to 10-fold) expansion of viable TILs was observed, when 0.1 μg/mL CD137-009-FEALxPD-L1-547-FEAR was added to cultures (FIG. 7A). When analyzed separately, a strong effect on CD3⁺CD8⁺ T cell expansion was observed, which was significant at 0.1 μg/mL CD137-009-FEALxPD-L1-547-FEAR (FIG. 7B; 7.4-fold expansion over control). CD3⁺CD4⁺ T cells were only slightly expanded, and their expansion was not significant compared to cultures without antibody (FIG. 7C). The most prominent TIL expansion was seen for CD3⁻CD56⁺ NK cells (FIG. 7D; up to 64-fold expansion over control), which was significant at 0.1 μg/mL CD137-009-FEALxPD-L1-547-FEAR.

### Example 11: Pharmacodynamic Evaluation of GEN1046 in Peripheral Blood in Patients with Advanced Solid Tumors

To investigate the biological activity of GEN1046 at various dose levels in patients with advanced tumors, blood and serum samples were collected at baseline and at multiple timepoints on treatment. Based on the mechanism of action of GEN1046, it was anticipated that dose levels with biological activity will modulate circulating levels of interferon-γ (IFN-γ) and interferon-gamma-inducible protein 10 (IP-10) and induce proliferation of peripheral CD8 T cells.

To determine serum levels of IFN-γ and IP-10, serum samples were collected from patients at baseline and at multiple timepoints post administration of GEN1046 in cycle 1 and cycle 2 (days 1 [2 h and between 4-6 h post-administration], 2, 3, 8, and 15). Serum levels of IFN- and IP-10 were measured by a Meso Scale Discovery (MSD) multiplex immune-assay (cat. no. K15209G) following the manufacturer's instructions.

To measure peripheral modulation of immune cells subsets, immunophenotyping of peripheral blood was conducted in whole blood collected in EDTA tubes at baseline and at multiple timepoints post GEN1046 administration in cycle 1 and cycle 2 (days 2, 3, 8 and 15). 100 μL of whole blood was added to fluorochrome-conjugated monoclonal antibodies that bind specifically to cell surface antigens: CD45RA-FITC (clone LEU-18, BD Biosciences cat. no. 335039), CCR7-BV510 (clone 3D12, BD Biosciences, cat. no. 563449), CD8-PerCP-Cy5.5 (clone RPA- T8, BD Biosciences, cat. no. 560662). After incubation on ice, the stained samples were treated with FACS Lysing Solution (BD Biosciences, Catalog No 349202) to lyse erythrocytes. Excess antibody and cell debris were removed by washing with Stain Buffer (BD Biosciences, cat. no. 554656). Following lyse/wash, cells were fixed and permeabilized by incubation with Permeabilizing Solution 2 buffer (BD Biosciences, cat. no. 340973). Next, cells were washed and resuspended in Stain Buffer and incubated on ice with antibody to Ki67 (BV421 B56, BD Biosciences, cat. no. 562899) to detect proliferating cells. After incubation, excess antibody was removed by washing with Stain Buffer. Cells were resuspended in Stain Buffer and acquired on a BD FACSCanto™ II flow cytometer (Becton Dickinson) within 1 hour of staining.

Administration of GEN1046 to cancer patients resulted in modulation of circulating levels of IFN-γ and IP-10 and proliferating effector memory CD8 T cells (Table 5 and FIG. 8). In the preliminary data set shown in Table 5, levels of IFN-γ increased more than 2-fold in the first treatment cycle across all dose levels tested. Maximal increases were detected at the 50 mg and 80 mg dose levels, and most of the patients in the 80 mg cohort (75%) had fold-increase >2 (Table 5). GEN1046 also elicited proliferation of effector memory CD8+ T cells as measured by an increase in the frequency of Ki67⁺CD8⁺CD45RA⁻CCR7⁻ T cells. Comparable to the changes observed with modulation of circulating levels of IFN-γ, maximal and more consistent modulation of proliferating CD8⁺ effector memory T cells was observed in patients in the 80 mg cohort. Particularly in the 400 mg cohort the magnitude of the changes in both the circulating levels of IFN-γ and proliferating effector memory CD8 T cells were lower compared to the 25-200 mg cohorts. These results showed that GEN1046 elicited an immune response characterized by modulation of immune effector cells and soluble factors critical for the generation of antitumor immune responses, with responses of greater magnitude at the 80 mg dose level.

In the data set shown in FIG. 8, an increase in IFN-γ and IP-10 was observed in the first treatment cycle at dose levels ≤200 mg (FIG. 8A-B) Although also an increase in IFN-γ and IP-10 was observed at dose levels ≥400 mg, the maximal fold change from baseline during the first treatment cycle was significantly lower compared to the lower dose levels. GEN1046 also elicited proliferation of total CD8⁺ T cells and effector memory CD8+ T cells as measured by an increase in the frequency of Ki67⁺CD8⁺ T cells and Ki67⁺CD8⁺CD45RA⁻CCR7⁻ T cells (FIG. 8C-D). Comparable to the changes observed with modulation of circulating levels of IFN-γ and IP-10, maximal and more consistent modulation of proliferating CD8⁺ effector memory T cells was observed in patients treated with dose levels ≤200 mg, In the >400 mg cohorts the magnitude of the changes in proliferating effector memory CD8 T cells and total CD8 T cells were significantly lower compared to the 25-200 mg cohorts. These results showed that GEN1046 elicited an Immune response characterized by modulation of immune effector cells and soluble factors critical for the generation of antitumor immune responses, with responses of greater magnitude at the ≤200 mg dose levels.

TABLE 5
GEN1046 Modulation of Peripheral Pharmacodynamic
Endpoints in cancer patients: Peak Fold-change from
Baseline during Cycle 1 by Dose Level ^{a}

| | GEN1046 | GEN1046 | GEN1046 | GEN1046 | GEN1046 |
|---|---|---|---|---|---|
| | 25 mg | 50 mg | 80 mg | 200 mg | 400 mg |
| Interferon-γ^{b} | | | | | |
| n | 4 | 4 | 8 | 8 | 6 |
| Min | 1.17 | 1.06 | 1.45 | 1.47 | 1.18 |
| Q1 | 2.05 | 1.89 | 2.82 | 2.35 | 1.32 |
| Median | 3.90 | 4.63 | 4.49 | 3.48 | 2.56 |
| Q3 | 9.99 | 6.90 | 5.94 | 4.89 | 3.37 |
| Max | 15.11 | 7.27 | 12.17 | 5.20 | 102.08 |
| Proliferating | | | | | |
| Effector | | | | | |
| Memory | | | | | |
| CD8 T cells^{c} | | | | | |
| n | 3 | 2 | 8 | 8 | 7 |
| Min | 2.00 | 2.00 | 1.00 | 0.67 | 1.00 |
| Q1 | 2.00 | 2.00 | 2.00 | 1.40 | 1.06 |
| Median | 2.00 | 2.50 | 3.42 | 2.83 | 1.50 |
| Q3 | 3.50 | 3.00 | 9.75 | 5.25 | 2.00 |
| Max | 5.00 | 3.00 | 31.40 | 6.67 | 7.00 |

Preliminary data as of 27 Jan. 2020.
n: number of patients per dose cohort; Min: lowest measured value; Q1: 25th percentile; Q3: 75th percentile; Max: maximum measured value.
^{a} Pharmacodynamic assessments, including changes in circulating levels of interferon-gamma and effector memory T cells, were conducted using blood samples from patients with advanced solid tumors enrolled in the dose escalation phase of an open-label, multi-center safety trial of GEN1046 (NCT03917381).
^{b}Circulating levels of interferon-gamma were measured in serum samples at baseline, and at multiple timepoints post administration of GEN1046 in cycle 1 and cycle 2 (days 1 [2 h and between 4-6 h post-administration], 2, 3, 8, and 15). Interferon-gamma levels in serum samples were determined by Meso Scale Discovery (MSD) multiplex immune assay.
^{c}Immunophenotyping of peripheral blood was conducted in whole blood collected at baseline and at multiple timepoints post administration of GEN1046 in cycle 1 and cycle 2 (days 2, 3, 8 and 15). The frequency of proliferating (Ki67⁺) effector memory CD8 T cells (CD8⁺CD45RA⁻CCR7⁻ T cells) were assessed in whole blood samples by flow cytometry.

### Example 12: Clinical Trial

### Trial Design

Clinical trial on GCT1046-01 (ClinicalTrials.gov Identifier: NCT03917381) was designed as a two-part trial, including an ongoing dose escalation part and a planned expansion part.

The trial was designed as an open-label, multi-center, Phase I/IIa safety trial of GEN1046 (DuoBody®-PD-L1x4-1BB). The trial consists of 2 parts; a First-in-Human (FIH) dose escalation (Phase I) and an expansion (Phase IIa). FIG. 9 shows a schematic representation of the clinical trial design.

GEN1046 has the following amino acid sequences:

CD137-binding arm; heavy and light chain sequences, respectively:

CD137-009-HC7LC2-FEAR
VH_CD137-009-H7 (SEQ ID NO: 1),
IgG1-FEAR-Fc (SEQ ID NO: 34)
VL_CD137-009-L2 (SEQ ID NO: 5),
Kappa-C (SEQ ID NO: 21)

PD-L1-binding arm; heavy and light chain sequences, respectively:

PD-L1-547-FEAL
VH-PD-L1-547 (SEQ ID NO: 8),
IgG1-FEAL-Fc (SEQ ID NO: 35)
VL-PD-L1-547 (SEQ ID NO: 12),
Lambda-C (SEQ ID NO: 22)

### Dose Escalation

The dose escalation was designed to evaluate GEN1046 in subjects with solid malignant tumors to determine the maximum tolerated dose (MTD) or maximum administered dose (MAD) and/or the recommended phase 2 dose (RP2D). The expansion further evaluated the safety, tolerability, PK, and anti-tumor activity of the selected dose(s) in select solid tumors.

For dose escalation, subject was required to be a man or woman ≥18 years of age and was required to have measurable disease according to RECIST 1.1.

Subjects was required to have a histologically or cytologically confirmed non-CNS solid tumor that was metastatic or unresectable and for whom there was no available standard therapy likely to confer clinical benefit, or subjects who are not candidates for such available therapy, and for whom, in the opinion of the investigator, experimental therapy with GEN1046 could be beneficial. In the dose escalation, subjects received one infusion of GEN1046 every third week (1Q3W) until protocol defined treatment discontinuation criteria are met; e.g. Radiographic disease progression or clinical progression. GEN1046 was be administered using i.v. infusion over a minimum of 60 minutes on Day 1 of each 3-week treatment cycle (21 days). The concept of the design of the trial is shown in FIG. 9.

The 1Q3W dose escalation was designed to potentially (dependent on data collected during the trial) evaluate GEN1046 at 7 main dose levels: 25, 80, 200, 400, 800, 1200 and 1600 mg fixed, and 6 optional intermediate dose levels 50, 140, 300, 600, 1000 and 1400 mg fixed.

The recommended phase 2 dose (RP2D) was based on a review of the available safety and dosing information and could be lower than the maximum tolerated dose (MTD).

### Expansion

The aim of the expansion is to provide further data on the safety, tolerability, MoA, PK and anti-tumor activity of the selected dose/schedule.

The expansion was designed to enroll subjects with relapsed or refractory, advanced and/or metastatic non-small cell lung cancer (NSCLC), endometrial carcinoma, urothelial carcinoma (UC), triple-negative breast cancer (TNBC), squamous cell carcinoma of the head and neck (SCCHN), or cervical cancer who are no longer candidates for standard therapy (if subjects had access and were eligible for the respective treatments), and for whom, in the opinion of the investigator, experimental therapy with GEN1046 may be beneficial. An overview of the expansion cohorts is provided in Table 6.

TABLE 6
Expansion cohorts

| Cohort | | Cancer | Sub- | Prior | Trial |
|---|---|---|---|---|---|
| No. | n | Type | cohort | Treatment | Treatment |
| EC1 | 140 | NSCLC | | Prior CPI | GEN1046 |
| | | | | treatment | 100 mg 1Q3W |
| EC2 | 40 | NSCLC | | PD-1/L1 naive | GEN1046 |
| | | | | | 100 mg 1Q3W |
| EC3 | 40 | UC | | Prior CPI | GEN1046 |
| | | | | treatment | 100 mg 1Q3W |
| EC4 | 40 | Endometrial | | PD-1/L1 naive | GEN1046 |
| | | cancer | | | 100 mg 1Q3W |
| EC5 | 40 | TNBC | 5a | Prior CPI | GEN1046 |
| | | | | treatment | 100 mg 1Q3W |
| | | | 5b | PD-1/L1 naive | GEN1046 |
| | | | | | 100 mg 1Q3W |
| EC6 | 40 | SCCHN | 6a | Prior CPI | GEN1046 |
| | | | | treatment | 100 mg 1Q3W |
| | | | 6b | PD-1/L1 naive | GEN1046 |
| | | | | | 100 mg 1Q3W |
| EC7 | 40 | Cervical | | PD-1/L1 naive | GEN1046 |
| | | cancer | | | 100 mg 1Q3W |

The expansion cohorts enroll patients with the following inclusion criteria:

### Expansion Cohort 1 (NSCLC): PD-1/L1 Pre-Treated

NSCLC subjects who have received up to 4 prior systemic treatment regimens (maintenance treatment is considered being part of one treatment line) for metastatic disease with radiographic disease progression on or after last prior treatment.
NSCLC subjects of any histology may be enrolled. Subjects with a histological or cytological diagnosis of non-squamous NSCLC must not have an epidermal growth factor receptor (EGFR)-sensitizing mutation and/or anaplastic lymphoma kinase (ALK) translocation/c-ROS oncogene 1 (ROS1) rearrangement. EGFR-sensitizing mutations are those mutations that are amenable to treatment with an approved tyrosine kinase inhibitor (TKI). Documentation of EGFR and ALK status should be available per local assessment. If documentation of EGFR and ALK status is unavailable, sponsor medical monitor approval is required prior to enrollment.
Subjects should have received platinum-based therapy (or alternative chemotherapy due to platinum ineligibility, e.g., a gemcitabine-containing regimen).
Subjects must have received prior treatment with a PD-1/L1 inhibitor alone or in combination and must have radiographic disease progression on treatment. Sponsor approval is required for subjects with a best overall response (BOR) of stable disease (SD) or progressive disease (PD) on a checkpoint inhibitor (CPI) containing regimen with a treatment duration of up to 16 weeks.
Local results from the most recent PD-L1 test must be provided prior to enrollment. If local PD-L1 test results are unavailable, sponsor approval for enrollment is required.

### Expansion Cohort 2 (NSCLC)—PD-1/L1 Naive

NSCLC subjects of any histology may be enrolled. NSCLC subjects who have received up to 4 prior systemic treatment regimens (maintenance treatment is considered being part of one treatment line) for metastatic disease with radiographic disease progression on or after last prior treatment.
Subjects with a histological or cytological diagnosis of non-squamous NSCLC must not have an EGFR-sensitizing mutation and/or ALK translocation/ROS1 rearrangement. EGFR-sensitizing mutations are those mutations that are amenable to treatment with an approved TKI. Documentation of EGFR and ALK status should be available per local assessment. If documentation of EGFR and ALK status is unavailable, sponsor medical monitor approval is required prior to enrollment.
Subjects should have received platinum-based therapy (or alternative chemotherapy due to platinum ineligibility, eg, a gemcitabine-containing regimen).
Subjects must not have received prior treatment with a PD-1/L1 inhibitor

### Expansion Cohort 3 (UC)

UC (of the bladder, ureter, urethra, or renal pelvis) subjects with predominantly transitional-cell features on histology who have received up to 4 prior systemic treatment regimens (maintenance treatment is considered being part of one treatment line) for locally advanced/metastatic disease with radiographic disease progression on or after last prior treatment.
Subjects must have received prior treatment with a PD-1/L1 inhibitor alone or in combination and must have radiographic disease progression on treatment. Sponsor approval is required for subjects with a best overall response (BOR) of SD or PD on a CPI containing regimen with a treatment duration of up to 16 weeks.
Local results from the most recent PD-L1 test should be provided prior to enrollment (if available).

### Cohort 3a: For Subjects Who Are Eligible to Receive Platinum-Based Therapy

Subjects must have received platinum-based chemotherapy.

### Cohort 3b: For Subjects Ineligible to Receive Platinum-Based Therapy

Subjects must not be eligible for any platinum-based or any cisplatin-containing chemotherapy.

### Expansion Cohort 4 (Endometrial Cancer)

Endometrial cancer subjects who have received up to 4 prior systemic treatment regimens (maintenance treatment is considered being part of one treatment line) for advanced/metastatic disease with radiographic disease progression on or after last prior treatment.
Subjects must have epithelial endometrial histology including: endometrioid, serous, squamous, clear-cell carcinoma, or carcinosarcoma.
Note: Sarcomas and mesenchymal endometrial cancer are excluded.
Subjects must not have received prior treatment with a PD-1/L1 inhibitor (established local label/access need to be respected).
Local results of the most recent deficient mismatch repair (dMMR) or microsatellite instability (MSI) status as per local assessment should be provided prior to enrollment (if available).

### Expansion Cohort 5 (TNBC)

TNBC defined as human epidermal growth factor receptor 2 (HER2)-negative (HER2 is negative by fluorescence in situ hybridization) assay (non-amplified ratio of HER2 to CEP17<2.0 single probe average HER2 gene copy number<4 signals/cell) or alternatively HER2 protein expression by immunohistochemistry (IHC) result is 1+ negative or IHC 0− negative and estrogen receptor and progesterone receptor negative status (defined as <1% of cells expressing hormonal receptors via IHC analysis) as per local assessment. Subjects who have received up to 4 prior systemic treatment regimens including but not limited to anthracycline-, taxane-, antimetabolite-, or microtubule inhibitor-containing regimens (maintenance treatment is considered being part of one treatment line) for locally advanced/metastatic disease with radiographic disease progression on or after last prior treatment. Local pathological confirmation of triple-negative disease is required prior to trial entry.
Subjects with a prior history of a breast cancer with a different phenotype must have confirmation of TNBC from a biopsy obtained after the subject's last prior systemic therapy.
Local results of the most recent dMMR or MSI status as per local assessment should be provided prior to enrollment (if available).

### Cohort 5a—Subjects Who Have Received Prior Treatment with a PD-1/L1 Inhibitor

Subjects must have received prior treatment with a PD-1/L1 inhibitor alone or in combination and must have radiographic disease progression on treatment. Sponsor approval is required for subjects with a BOR of SD or PD on a CPI containing regimen with a treatment duration of up to 16 weeks.
Local results from the most recent PD-L1 test should be provided prior to enrollment (if available).

### Cohort 5b—Subjects Who Have Not Received Prior Treatment with a PD-1/L1 Inhibitor

Subjects must not have received prior treatment with a PD-1/L1 inhibitor (established local label/access need to be respected).

### Expansion Cohort 6 (SCCHN)

Recurrent or metastatic SCCHN (oral cavity, pharynx, larynx) subjects who have received up to 4 prior systemic treatment regimens for recurrent/metastatic disease with radiographic PD on or after last prior treatment (maintenance treatment is considered being part of one treatment line).
Subjects must have disease progression on or after prior therapy with platinum-based chemotherapy (alternative combination chemotherapy is acceptable if the subject's platinum ineligibility status is documented).

### Cohort 6a—Subjects Who Have Received Prior Treatment with a PD-1/L1 Inhibitor

Subjects must have received prior treatment with a PD-1/L1 inhibitor alone or in combination and must have radiographic disease progression on treatment. Sponsor approval is required for subjects with a BOR of SD or PD on a CPI containing regimen with a treatment duration of up to 16 weeks.
Local results from the most recent PD-L1 test should be provided prior to enrollment (if available).

### Cohort 6b—Subjects Who Have Not Received Prior Treatment with a PD-1/L1 Inhibitor

Subjects must not have received prior treatment with a PD-1/L1 inhibitor (established local label/access need to be respected).

### Expansion Cohort 7 (Cervical Cancer)

Cervical cancer subjects who have received up to 4 prior systemic treatment regimens including chemotherapy in combination with bevacizumab (according to the applicable labeling) unless the subject is ineligible for bevacizumab according to local standards (chemotherapy administered in the adjuvant or neoadjuvant setting, or in combination with radiation therapy should not be counted as a prior line of therapy) for recurrent/metastatic disease with radiographic disease progression on or after last prior treatment.
Subjects must have cervical cancer of squamous cell, adenocarcinoma, or adenosquamous histology.
Subjects must not have received prior treatment with a PD-1/L1 inhibitor (established local label/access need to be respected).

### Results

### Dose Escalation

The following preliminary results were obtained during dose escalation. Table 7 shows Best Overall Response (RECIST v1.1) by Dose Level upon enrolment and dosing of a total of 30 patients (Data Extraction Date: 3 Feb. 2020).

Tables 8 and 9 show Objective Response Rate and Confirmed Objective Response Rate, respectively (RECIST v1.1) by Dose Level upon enrolment and dosing of a total of 61 patients (Data cut-off: Oct. 12, 2020).

Best percent change from baseline in tumor size in all patients is shown in FIG. 10. Disease control occurred in 40/61 (65.6%) patients in the dose escalation phase. Partial response (PR) was achieved in four patients with triple-negative breast cancer, ovarian cancer, or non-small cell lung cancer (NSCLC); 36 patients maintained stable disease.

Clinical activity observed in patients with NSCLC (best change from baseline in tumor size) is show in in FIG. 11 (Data cut-off: Oct. 12, 2020). Of six patients with NSCLC, all of whom had received prior checkpoint immunotherapy, two achieved unconfirmed PR, two maintained stable disease, and two experienced progressive disease.

TABLE 7
Best Overall Response (RECIST v1.1) by Dose Level.

| | 25 mg | | 50 mg | | 80 mg | | 140 mg | | 200 mg | | 400 mg | | Total | |
|---|---|---|---|---|---|---|---|---|---|---|---|---|---|---|
| | (n = 2) | | (n = 5) | | (n = 8) | | (n = 1) | | (n = 8) | | (n = 6) | | (n = 30) | |
| Complete | 0 | (0) | 0 | (0) | 0 | (0) | 0 | (0) | 0 | (0) | 0 | (0) | 0 | (0) |
| Response | | | | | | | | | | | | | | |
| Partial | 0 | (0) | 0 | (0) | 2¹ | (25) | 0 | (0) | 1¹ | (12.5) | 0 | (0) | 3 | (10) |
| Response | | | | | | | | | | | | | | |
| Stable Disease | 0 | (0) | 3 | (60) | 5 | (62.5) | 0 | (0) | 5 | (62.5) | 6 | (100) | 19 | (63.3) |
| Progressive | 2 | (100) | 2 | (40) | 1 | (12.5) | 1 | (100) | 2 | (25) | 0 | (0) | 8 | (26.6) |
| Disease | | | | | | | | | | | | | | |

¹uPR

TABLE 8
Objective Response Rate - dose escalation

| | Total | 25 mg | 50 mg | 80 mg | 100 mg | 140 mg | 200 mg | 400 mg | 800 mg | 1200 mg |
|---|---|---|---|---|---|---|---|---|---|---|
| N | 61 | 4 | 5 | 9 | 6 | 6 | 9 | 9 | 9 | 4 |
| Best Overall Response | | | | | | | | | | |
| CR (Complete Response) | 0 | 0 | 0 | 0 | 0 | 0 | 0 | 0 | 0 | 0 |
| PR (Partial Response) | 4 | 0 | 0 | 2 | 1 | 0 | 1 | 0 | 0 | 0 |
| | (6.6%) | | | (22.2%) | (16.7%) | | (11.1%) | | | |
| SD (Stable Disease) | 36 | 1 | 3 | 6 | 3 | 3 | 5 | 7 | 5 | 3 |
| | (59.0%) | (25.0%) | (60.0%) | (66.7%) | (50.0%) | (50.0%) | (55.6%) | (77.8%) | (55.6%) | (75.0%) |
| PD (Progressive Disease) | 14 | 2 | 2 | 1 | 0 | 2 | 2 | 2 | 2 | 1 |
| | (23.0%) | (50.0%) | (40.0%) | (11.1%) | | (33.3%) | (22.2%) | (22.2%) | (22.2%) | (25.0%) |
| NE (Not Evaluable) | 7 | 1 | 0 | 0 | 2 | 1 | 1 | 0 | 2 | 0 |
| | (11.5%) | (25.0%) | | | (33.3%) | (16.7%) | (11.1%) | | (22.2%) | |
| Objective Response (CR + PR) | 4 | 0 | 0 | 2 | 1 | 0 | 1 | 0 | 0 | 0 |
| Rate | (6.6%) | | | (22.2%) | (16.7%) | | (11.1%) | | | |
| Disease Control (PR + PR + SD) | 40 | 1 | 3 | 8 | 4 | 3 | 6 | 7 | 5 | 3 |
| Rate | (65.6%) | (25.0%) | (60.0%) | (88.9%) | (66.7%) | (50.0%) | (66.7%) | (77.8%) | (55.6%) | (75.0%) |

TABLE 9
Confirmed Response Rate - dose escalation

| | Total | 25 mg | 50 mg | 80 mg | 100 mg | 140 mg | 200 mg | 400 mg | 800 mg | 1200 mg |
|---|---|---|---|---|---|---|---|---|---|---|
| N | 61 | 4 | 5 | 9 | 6 | 6 | 9 | 9 | 9 | 4 |
| Confirmed Best Overall | | | | | | | | | | |
| Response | | | | | | | | | | |
| CR (Complete Response) | 0 | 0 | 0 | 0 | 0 | 0 | 0 | 0 | 0 | 0 |
| PR (Partial Response) | 2 | 0 | 0 | 1 | 1 | 0 | 0 | 0 | 0 | 0 |
| | (3.3%) | | | (11.1%) | (16.7%) | | | | | |
| SD (Stable Disease) | 38 | 1 | 3 | 7 | 3 | 3 | 6 | 7 | 5 | 3 |
| | (62.3%) | (25.0%) | (60.0%) | (77.8%) | (50.0%) | (50.0%) | (66.7%) | (77.8%) | (55.6%) | (75.0%) |
| PD (Progressive Disease) | 14 | 2 | 2 | 1 | 0 | 2 | 2 | 2 | 2 | 1 |
| | (23.0%) | (50.0%) | (40.0%) | (11.1%) | | (33.3%) | (22.2%) | (22.2%) | (22.2%) | (25.0%) |
| NE (Not Evaluable) | 7 | 1 | 0 | 0 | 2 | 1 | 1 | 0 | 2 | 0 |
| | (11.5%) | (25.0%) | | | (33.3%) | (16.7%) | (11.1%) | | (22.2%) | |
| Confirmed Objective | 2 | 0 | 0 | 1 | 1 | 0 | 0 | 0 | 0 | 0 |
| Response (CR + PR) Rate | (3.3%) | | | (11.1%) | (16.7%) | | | | | |
| Confirmed Disease Control | 40 | 1 | 3 | 8 | 4 | 3 | 6 | 7 | 5 | 3 |
| (PR + PR + SD) Rate | (65.6%) | (25.0%) | (60.0%) | (88.9%) | (66.7%) | (50.0%) | (66.7%) | (77.8%) | (55.6%) | (75.0%) |

### Expansion

Expansion cohort 1: As of Jan. 29, 2021, 39 patients had been dosed in expansion cohort 1, which includes patients with advanced/metastatic PD-1/L1 pre-treated NSCLC. Of the 39 patients, 31 were evaluable for efficacy at the time of data cutoff i.e. had either at least one post baseline scan or had discontinued treatment. Six of the 31 efficacy evaluable patients experienced best overall response of either confirmed or unconfirmed PR and 7 patients experienced best overall response of SD (FIG. 13).

Expansion cohort 2: As of Jan. 29, 2021, 11 patients had been dosed in expansion cohort 2, which includes patients with advanced/metastatic PD-1/L1 naïve NSCLC. Of the 11 patients, 10 were evaluable for efficacy at the time of data cutoff i.e. had either at least one post baseline scan or had discontinued treatment. None of the 10 efficacy evaluable patients experienced best overall response of PR whereas 5 patients experienced best overall response of SD (FIG. 14).

Expansion cohort 3: As of Jan. 29, 2021, 13 patients had been dosed in expansion cohort 3, which includes patients with advanced/metastatic PD-1/L1 pre-treated urothelial carcinoma. Of the 13 patients, 9 were evaluable for efficacy at the time of data cutoff i.e. had either at least one post baseline scan or had discontinued treatment. None of the 10 efficacy evaluable patients experienced best overall response of PR whereas 4 patients experienced best overall response of SD (FIG. 15).

Expansion cohort 4: As of Jan. 29, 2021, 21 patients had been dosed in expansion cohort 4, which includes patients with advanced/metastatic, PD-1/L1 naïve endometrial carcinoma. Of the 21 patients, 17 were evaluable for efficacy at the time of data cutoff i.e. had either at least one post baseline scan or had discontinued treatment. One of the 17 efficacy evaluable patients experienced best overall response of PR whereas 7 patients experienced best overall response of SD (FIG. 16).

Expansion cohort 5: As of Jan. 29, 2021, 20 patients had been dosed in expansion cohort 5, which includes patients with advanced/metastatic TNBC. Of the 20 patients, 15 were evaluable for efficacy at the time of data cutoff i.e. had either at least one post baseline scan or had discontinued treatment. One of the 15 efficacy evaluable patients experienced best overall response of PR whereas 5 patients experienced best overall response of SD (FIG. 17).

Expansion cohort 6: As of Jan. 29, 2021, 22 patients had been dosed in expansion cohort 6, which includes patients with advanced/metastatic SCCHN. Of the 22 patients, 18 were evaluable for efficacy at the time of data cutoff i.e. had either at least one post baseline scan or had discontinued treatment. Two of the 18 efficacy evaluable patients experienced best overall response of PR whereas 6 patients experienced best overall response of SD (FIG. 18).

Expansion cohort 7: As of Jan. 29, 2021, 16 patients had been dosed in expansion cohort 7, which includes patients with advanced/metastatic, PD-1/L1 naïve cervical cancer. Of the 16 patients, 11 were evaluable for efficacy at the time of data cutoff i.e. had either at least one post baseline scan or had discontinued treatment. One of the 11 efficacy evaluable patients experienced best overall response of PR whereas 7 patients experienced best overall response of SD (FIG. 19).

Across expansion cohorts, progression-free survival (PFS) was longer in subjects having received prior treatment with checkpoint inhibitor (FIG. 20).

For patients combined across CPI pre-treated expansion cohorts, clinical response to GEN1046 therapy in checkpoint inhibitor pre-treated NSCLC subjects associates with time from last prior anti-PD-1 therapy (FIG. 21).
NSCLC subjects with benefit on GEN1046 therapy showed a trend for more recent treatment with last anti-PD-1 agent
Shorter time since anti-PD-1 agent containing therapy may suggest residual anti-PD-1 activity is facilitating response to GEN1046. Supportive of this, patients treated with anti-PD-1 agents in the clinic exhibit long-term PD-1 receptor occupancy by the therapeutic antibody which can last for more than 200 days (Brahmer et al., JCO 2010; 28(19): 3167-3175). Having therapeutic a-PD-1 agent still bound to the PD-1 receptors may in turn lead to a larger number of free PD-L1 molecules being available for binding to GEN1046.
Presence of residual a-PD-1 activity may also allow for more complete blockade of the PD-1 pathway (blocking interaction of PD-1 with both PD-L1 and PD-L2), which may be important for the biological activity of GEN1046 in the post-CPI setting.
More recent anti-PD-1 treatment may have direct impact on the tumor microenvironment, for example by initiating an anti-tumor immune response which can be enhanced by GEN1046 if it is given immediately or soon after progression on the anti-PD-1 containing therapy.

Responders presented with “low” PD-1+CD8 T cell frequency, which may reflect receptor occupancy (RO) by prior a-PD-1 treatment.

Conversely, non-responders presented with generally high PD-1+CD8 T cell frequency which may indicate a more exhausted phenotype.

### Conclusions

GEN1046 is a first-in-class, next-generation, PD-L1x4-1BB bispecific antibody with an acceptable safety profile and encouraging early clinical activity, unlike the existing 4-1BB agonists.

In the dose escalation phase of this phase I/IIa study, GEN1046 demonstrated a manageable safety profile and preliminary clinical activity in a heavily pretreated population with advanced solid tumors.

Most adverse events were mild to moderate; treatment-related Grade 3 transaminase elevations resolved with corticosteroids. No treatment-related bilirubin increases or Grade 4 transaminase elevations were observed. Six patients had dose limiting toxicities (DLTs); Maximum tolerated dose (MTD) was not reached.

Clinical benefit across different dose levels was observed in patients, including those resistant to prior immunotherapy and those with tumors typically less sensitive to immune checkpoint inhibitors (ICIs).

Disease control was achieved in 65.6% of patients, including partial responses in triple negative breast cancer (1), ovarian cancer (1), and ICI pre-treated NSCLC (2).

Modulation of pharmacodynamic endpoints was observed across a broad range of dose levels demonstrating biological activity.

### Example 13: Pharmacokinetic/Pharmacodynamic Model

An integrated semi-mechanistic PK/PD (Pharmacokinetic/Pharmacodynamic) model was developed that assumes distribution of GEN1046 into central and peripheral PK compartments, as well as partitioning into tumor and lymph compartments. The model leverages PK and pharmacodynamic data as well as physiological parameters from literature for parameterizations of expressions of PD-L1 and 4-1BB, and T-cell trafficking into these cells. Model compartments consists of well-mixed 2- and 3-dimensional spaces and free drug transfer between all compartments. In addition, the model incorporates dynamic binding of GEN1046 to PD-L1 and 4-1BB to predict trimer (crosslinking to PD-L1 and 4-1BB) formation and receptor occupancy (RO) for PD-L1 and 4-1BB in tumor.

The semi-mechanistic PK/pharmacodynamic model shows that trimer formation in the tumor peaks at a GEN1046 regimen of 100 mg Q3W, which is expected to provide continuous 4-1BB activation and is selected as the activation dose for the first 2 cycles. In addition, based on available clinical pharmacodynamic data, higher magnitude and consistent modulation of peripheral pharmacodynamic endpoints (IFNy and proliferating Ki67+ effector memory CD8+ T cells) were seen at dose levels ≤200 mg. In the GCT1046-01 trial, clinical data from the expansion cohort showed that the dose of 100 mg Q3W resulted in responses within first 2 cycles.

In light of PK/pharmacodynamic modeling predictions and available clinical data, a dose of GEN1046 100 mg 1Q3W was selected as activation dose to be given for first 2 cycles that can lead to maximal trimer formation and average RO for PD-L1 (%) at reasonable levels.

A maintenance regimen of GEN1046 500 mg 1Q6W will be used after the first 2 cycles and is predicted to provide higher PD-L1 receptor occupancy over the dosing cycle and intermittent 4-1BB activation via engaging trimers to a lesser extent in comparison to 100 mg Q3W (FIG. 12). This dose is expected to provide improved duration of response. Further, GEN1046 at 500 mg Q6W is predicted to engage less trimers in liver compared to 100 mg Q3W and therefore may have a better safety profile.

### Example 14: Additional Expansion Cohort; Activation/Maintenance Dosing

The integrated semi-mechanistic physiologically based pharmacokinetics/pharmacodynamic model provided in Example 13 was used to predict trimer (crosslinking to PD-L1 and 4-1BB) formation and receptor occupancy (RO) for PD-L1 in tumors. The model was then used to explore the predicted in vivo trimer formation and PD-L1 RO at various dosing regimens. The model showed that trimer formation in tumor peaks at a dose of GEN1046 100 mg once every three weeks (1Q3W), which was selected as the activation dose for 2 cycles. This is followed by a maintenance dose of GEN1046 500 mg 1Q6W, which was predicted to provide higher PD-L1 receptor occupancy (RO) over the dosing cycle and intermittent 4-1BB activation via engaging trimers in comparison to 100 mg Q3W.

Two further expansion cohorts were designed to evaluate administration of GEN1046 at 100 mg 1Q3W as “activation dose” for 2 cycles, followed by a “maintenance dose” of 500 mg GEN1046 1Q6W.

The first expansion cohort enrolls metastatic check-point inhibitor- (CPI-) pretreated Non-Small Cell Lung Cancer (NSCLC) patients with the following inclusion criteria:
NSCLC subjects who have received up to 4 prior systemic treatment regimens (maintenance treatment is considered being part of one treatment line) for metastatic disease with radiographic disease progression on or after last prior treatment.
NSCLC subjects of any histology may be enrolled. Subjects with a histological or cytological diagnosis of non-squamous NSCLC must not have an epidermal growth factor receptor (EGFR)-sensitizing mutation and/or anaplastic lymphoma kinase (ALK) translocation/c-ROS oncogene 1 (ROS1) rearrangement. EGFR-sensitizing mutations are those mutations that are amenable to treatment with an approved tyrosine kinase inhibitor (TKI). Documentation of EGFR and ALK status should be available per local assessment. If documentation of EGFR and ALK status is unavailable, sponsor medical monitor approval is required prior to enrollment.
Subjects should have received platinum-based therapy (or alternative chemotherapy due to platinum ineligibility, e.g. a gemcitabine-containing regimen).
Subjects must have received prior treatment with a PD-1/L1 inhibitor alone or in combination and must have radiographic disease progression on treatment. Sponsor approval is required for subjects with a best overall response (BOR) of stable disease (SD) or progressive disease (PD) on a checkpoint inhibitor (CPI) containing regimen with a treatment duration of up to 16 weeks.
Local results from the most recent PD-L1 test must be provided prior to enrollment. If local PD-L1 test results are unavailable, sponsor approval for enrollment is required.

The second expansion cohort will enroll treatment naive metastatic NSCLC patients with the following inclusion criteria:
Subjects with metastatic NSCLC who have received no prior systemic treatment regimens for metastatic disease. Subjects must not have received prior treatment with a PD-1/L1 inhibitor. Subjects must have radiographic disease progression on or after last prior treatment. This is not required for subjects who have newly diagnosed disease.
Subjects with NSCLC of any histology may be enrolled. Subjects with a histological or cytological diagnosis of non-squamous NSCLC must not have an EGFR-sensitizing mutation and/or ALK translocation/ROS1 rearrangement. EGFR-sensitizing mutations are those mutations that are amenable to treatment with an approved TKI. Documentation of EGFR and ALK status should be available per local assessment. If documentation of EGFR and ALK status is unavailable, sponsor medical monitor approval is required prior to enrollment.
Subjects must have a PD-L1 expression result from the central laboratory available prior to C1D1 from a fresh tumor sample obtained by core-needle or excisional biopsy OR from resected tumor tissue at the time that metastatic disease was diagnosed. The following samples are not acceptable for this study: endobronchial ultrasound (EBUS)-guided samples, fine needle aspirates, cell blocks, cell pellets, clots, bone marrow, and cytological specimens.
Tumor demonstrates PD-L1 expression in ≥1% of tumor cells (TPS ≥1%) as assessed by IHC determined by central laboratory testing.

Example 15: Phase 2, Multicenter, Randomized, Open-Label Trial of GEN1046 in Subjects with Relapsed/Refractory Metastatic Non-Small Cell Lung Cancer after Treatment with Standard of Care Therapy with an Immune Checkpoint Inhibitor

This is a randomized, open-label trial evaluating the safety and efficacy of GEN1046 in adult subjects with relapsed/refractory metastatic NSCLC after treatment with CPI-containing therapy. The trial comprises a study arm A in which patients are treated with GEN1046 monotherapy, and the primary objective is to evaluate the anti-tumor activity (ORR) of GEN1046 as monotherapy. ORR is a well-established efficacy parameter for assessing anti-tumor activity in a proof-of-concept trial in NSCLC.

Arm A will test a regimen of an activation dose of GEN1046 (100 mg Q3W for 2 cycles) followed by a higher maintenance dose of GEN1046 (500 mg administered Q6W for the subsequent cycles), based on the following:
The semi-mechanistic PK/pharmacodynamic model shows that trimer formation in the tumor peaks at a GEN1046 regimen of 100 mg Q3W, which is expected to provide continuous 4-1BB activation and is selected as the activation dose for the first 2 cycles. In the GCT1046-01 trial, clinical data from the expansion cohort showed that the dose of 100 mg Q3W resulted in responses within the first 2 cycles.
A maintenance regimen of GEN1046 500 mg Q6W will be used after the first 2 cycles and is predicted to provide higher PD-L1 RO over the dosing cycle and intermittent 4-1BB activation via engaging trimers to a lesser extent in comparison to 100 mg Q3W. This dose is expected to provide improved duration of response (DOR). In addition, GEN1046 500 mg Q6W is expected to have an acceptable benefit-risk profile as the maximum concentration (Cₘₐₓ) after 500 mg Q6W will be lower compared to GEN1046 1200 mg Q3W, which is the highest tested dose in dose escalation phase. Doses of 25 to 1200 mg

Q3W evaluated in the escalation phase of the FIH trial were safe and generally well tolerated, and the MTD was not reached.

In Arm A, GEN1046 100 mg Q3W will be administered as a 30-minute IV infusion on Day 1 for the first 2 treatment cycles; thereafter, GEN1046 500 mg Q6W will be administered as a 30-minute IV infusion on Day 1 of the subsequent 6-week treatment cycles. No dose reduction is allowed for GEN1046.

### Key Inclusion Criteria

Subject must be at least 18 years of age.
Subject must have histologically or cytologically confirmed diagnosis of stage 4 NSCLC with at least 1 prior line of systemic therapy containing an anti-PD-1/PD-L1 mAb for metastatic disease listed below. Subjects must have demonstrated disease progression (PD) as defined by RECIST v1.1. For the subjects whose most recent anti-cancer therapy contained an anti-PD-1/PD-L1 mAb, their recent evidence of PD must be confirmed by a second assessment no less than 4 weeks from the date of the initial documented PD.
Note: Subject must have received at least 2 doses of an approved anti-PD-1/PD-L1 mAb approved in NSCLC.
   Subject has progressed during or after treatment with 1 anti-PD-1/PD-L1 mAb administered either as monotherapy, or as SOC combination (subjects who have received only anti-PD-1/PD-L1 mAb monotherapy as first-line therapy, are eligible for this study if the investigator determines treatment with platinum-containing chemotherapy is not appropriate, in line with local treatment guidelines) or;
   Subject has progressed during or after platinum doublet chemotherapy following an anti-PD-1/PD-L1 mAb or;
   Subject has progressed during or after an anti-PD-1/PD-L1 mAb following platinum doublet chemotherapy.
Subject must have a tumor PD-L1 expression result available prior to C1D1 demonstrating PD-L1 expression in ≥1% of tumor cells as assessed by a sponsor-designated central laboratory using the Dako PD-L1 IHC 22C3 pharmDx assay (TPS≥1%), or per site local assessment with the Dako PD-L1 IHC 22C3 pharmDx assay (TPS≥1%) or the VENTANA PD-L1 (SP263) assay (TC ≥1%) adhering to the manufacturer's instructions.
Note: Local PD-L1 result needs to be performed on fresh tumor tissue (obtained within 3 months prior to enrollment and after failure/stop of last prior treatment) or, if not feasible, archival tissue (obtained within 12 months prior to enrollment).
Subject must have measurable disease per RECIST v1.1 as assessed by the investigator.
Subject must have Eastern Cooperative Oncology Group (ECOG) performance status (PS) ≤1.
Subject must have life expectancy of at least 3 months.
Subject must have adequate organ and bone marrow function as described in the protocol.

### Key Exclusion Criteria

Documentation of known EGFR sensitizing mutations, KRAS, RET, ROS1, BRAF mutations, NTRK gene infusions, RET rearrangement, ALK gene rearrangements, high-level MET amplification, or METex 14 skipping. If documentation of mutation status is not available, for subjects with non-squamous histology or a mixed histology of non-squamous and squamous, a formalin-fixed, paraffin-embedded tumor tissue should be tested for biomarker panel analysis (which may include, but is not limited to, EGFR, ALK, ROS1, BRAF, KRAS mutations, RET rearrangement, or NTRK gene infusions, etc). Subjects must not be randomized until biomarker status is available in source documentation at the site.
Note: Subjects with tumors harboring such targetable mutations, gene rearrangements, or gene amplifications as described above may enroll in the trial, if such subjects have also received an approved targeted therapy for this indication assuming satisfactory fulfilment of all other eligibility criteria (especially, at least 1 prior line of systemic therapy containing an anti-PD-1/PD-L1 mAb for metastatic NSCLC disease).
Subject has been exposed to any of the following prior therapies:
   Prior treatment with docetaxel for NSCLC.
   Prior treatment with a 4-1BB (CD137) targeted agent, any type of antitumor vaccine, or autologous cell immunotherapy.
   Treatment with an anti-cancer agent within 28 days prior to GEN1046 administration.
Subject discontinued treatment due to disease progression within the first 6 weeks of an immune CPI containing treatment.

1. A method for reducing or preventing progression of a tumor or treating cancer in a subject, comprising administering to said subject a binding agent comprising a first antigen-binding region binding to human CD137, such as human CD137 consisting of the amino acid sequence set forth in SEQ ID NO: 24, and a second antigen-binding region binding to human PD-L1, such as human PD-L1 consisting of the amino acid sequence set forth in SEQ ID NO: 26, wherein
said binding agent is administered to the subject in a dosing schedule that comprises administration of Dose A in one or more treatment cycles and administration of dose B in one or more treatment cycles,
the amount of binding agent in Dose A being
a) about 0.3-2.5 mg/kg body weight, or about 25-200 mg in total; and/or
b) about 2.1×10⁻⁹-1.7×10⁻⁸ mol/kg body weight, or about 1.7×10⁻⁷-1.4×10⁻⁶ mol in total; and
the amount of binding agent in Dose B being
c) about 3.8-7.5 mg/kg body weight, or about 300-600 mg in total; and/or
d) about 2.6×10⁻⁸-5.1×10⁻⁸ mol/kg body weight, or about 2.0-4.1×10⁻⁶ mol in total.

2. The method according to any one of the preceding claims wherein the amount of said binding agent in Dose A is
0.4-2.3 mg/kg body weight or 30-180 mg in total, and/or 2.56×10⁻⁹-1.53×10⁻⁸ mol/kg body weight or 2.04×10⁻⁷-1.23×10⁻⁶ mol in total;
0.5-2.0 mg/kg body weight or 40-160 mg in total, and/or 3.41×10⁻⁹-1.36×10⁻⁸ mol/kg body weight or 2.73×10⁻⁷-1.09×10⁻⁶ mol in total;
0.6-1.9 mg/kg body weight or 50-150 mg in total, and/or 4.26×10⁻⁹-1.28×10⁻⁸ mol/kg body weight or 3.41×10⁻⁷-1.02×10⁻⁶ mol in total;
0.8-1.8 mg/kg body weight or 60-140 mg in total, and/or 5.11×10⁻⁹-1.19×10⁻⁸ mol/kg body weight or 4.09×10⁻⁷-9.54×10⁻⁷ mol in total;
0.9-1.6 mg/kg body weight or 70-130 mg in total, and/or 5.96×10⁻⁹-1.11×10⁻⁸ mol/kg body weight or 4.77×10⁻⁷-8.86×10⁻⁷ mol in total;
1-1.5 mg/kg body weight or 80-120 mg in total, and/or 6.81×10⁻⁹-1.02×10⁻⁸ mol/kg body weight or 5.45×10⁻⁷-8.18×10⁻⁷ mol in total;
1.1-1.4 mg/kg body weight or 90-110 mg in total, and/or 7.67×10⁻⁹-9.37×10⁻⁹ mol/kg body weight or 6.13×10⁻⁷-7.49×10⁻⁷ mol in total; or
1.2-1.3 mg/kg body weight or 95-105 mg in total, and/or 8.09×10⁻⁹-8.94×10⁻⁹ mol/kg body weight or 6.47×10⁻⁷-7.16×10⁻⁷ mol in total.

3. The method according to any one of the preceding claims, wherein the amount of said binding agent in Dose A is
a) about 1.25 mg/kg body weight, or about 100 mg in total; and/or
b) about 8.5×10⁻⁹ mol/kg body weight, or about 6.8×10⁻⁷ mol in total.

4. The method according to any one of the preceding claims, wherein the amount of binding agent in Dose B is
4.4-7.4 mg/kg body weight or 350-590 mg in total, and/or 2.98×10⁻⁸-5.03×10⁻⁸ mol/kg body weight or 2.39×10⁻⁶-4.02×10⁻⁶ mol in total;
5.0-7.25 mg/kg body weight or 400-580 mg in total, and/or 3.41×10⁻⁸-4.94×10⁻⁸ mol/kg body weight or 2.73×10⁻⁶-3.95×10⁻⁶ mol in total;
5.3-7.1 mg/kg body weight or 420-570 mg in total, and/or 3.58×10⁻⁸-4.86×10⁻⁸ mol/kg body weight or 2.86×10⁻⁶-3.88×10⁻⁶ mol in total;
5.4-7.0 mg/kg body weight or 430-560 mg in total, and/or 3.66×10⁻⁸-4.77×10⁻⁸ mol/kg body weight or 2.93×10⁻⁶-3.82×10⁻⁶ mol in total;
5.5-6.9 mg/kg body weight or 440-550 mg in total, and/or 3.75×10⁻⁸-4.69×10⁻⁸ mol/kg body weight or 3.00×10⁻⁶-3.75×10⁻⁶ mol in total;
5.6-6.8 mg/kg body weight or 450-540 mg in total, and/or 3.83×10⁻⁸-4.60×10⁻⁸ mol/kg body weight or 3.07×10⁻⁶-3.68×10⁻⁶ mol in total;
5.8-6.6 mg/kg body weight or 460-530 mg in total, and/or 3.92×10⁻⁸-4.51×10⁻⁸ mol/kg body weight or 3.13×10⁻⁶-3.61×10⁻⁶ mol in total;
5.9-6.5 mg/kg body weight or 470-520 mg in total, and/or 4.00×10⁻⁸-4.43×10⁻⁸ mol/kg body weight or 3.20×10⁻⁶-3.54×10⁻⁶ mol in total;
6.0-6.4 mg/kg body weight or 480-515 mg in total, and/or 4.09×10⁻⁸-4.39×10⁻⁸ mol/kg body weight or 3.27×10⁻⁶-3.51×10⁻⁶ mol in total;
6.1-6.4 mg/kg body weight or 490-510 mg in total, and/or 4.17×10⁻⁸-4.34×10⁻⁸ mol/kg body weight or 3.34×10⁻⁶-3.48×10⁻⁶ mol in total; or
6.2-6.3 mg/kg body weight or 495-505 mg in total, and/or 4.22×10⁻⁸-4.30×10⁻⁸ mol/kg body weight or 3.37×10⁻⁶-3.44×10⁻⁶ mol in total.

5. The method according to any one of the preceding claims, wherein the amount of binding agent in Dose B is
a) about 6.25 mg/kg body weight, or about 500 mg in total; and/or
b) about 4.3×10⁻⁸ mol/kg body weight, or about 3.4×10⁻⁶ mol in total.

6. The method according to any one of the preceding claims, wherein said dosing schedule comprises administration of Dose A in one or more treatment cycles, followed by administration of dose B in one or more treatment cycles.

7. The method according to any one of the preceding claims, wherein Dose A is administered once in each treatment cycles, such as on day 1 in each treatment cycle.

8. The method according to any one of the preceding claims, wherein Dose B is administered once in each treatment cycles, such as on day 1 in each treatment cycle.

9. The method according to any one of the preceding claims, wherein Dose A is administered in one or more three-week/21 day treatment cycles, such as 2, 3, 4 or 5 three week/21 day treatment cycles.

10. The method according to claim 9, wherein Dose A is administered once in each three-week/21-day treatment cycle (Q3W).

11. The method according to claim 9 or 10, wherein Dose A is administered on day 1 in each of said one or more three-week/21-day treatment cycles.

12. The method according to any one of the preceding claims, wherein Dose B is administered in one or more 6-week/42-day treatment cycles.

13. The method according to claim 12, wherein Dose B is administered once in each of said one or more 6-week/42-day treatment cycles (Q6W).

14. The method according to claim 12 or 13, wherein Dose B is administered on day 1 in each of said one or more 6-week/42-day treatment cycles.

15. The method according to any one of the preceding claims, wherein said dosing schedule comprises administration of Dose A in two (2) treatment cycles, followed by administration of dose B in one or more treatment cycles.

16. The method according to any one of the preceding claims, wherein said dosing schedule comprises administration of Dose A, followed by administration of dose B until complete tumor regression or disease progression.

17. The method according to any one of the preceding claims, comprising collecting whole blood samples and assessing PD-L1 receptor occupancy by the binding agent.

18. The method of to any one of the preceding claims, wherein the binding agent is administered by systemic administration.

19. The method of any one of the preceding claims, wherein the binding agent is administered by intravenous injection or infusion.

20. The method according to any one of the preceding claims, wherein each dose is infused over a minimum of 30 minutes, such as over a minimum of 60 minutes, a minimum of 90 minutes, a minimum of 120 minutes or a minimum of 240 minutes.

21. The method according to any one of the preceding claims, wherein
a) the first antigen-binding region comprises a heavy chain variable region (VH) comprising the complementarity determining region 1 (CDR1), complementarity determining region 2 (CDR2), and complementarity determining region 3 (CDR3) sequences of SEQ ID NO: 1, and a light chain variable region (VL) comprising the complementarity determining region1 (CDR1), complementarity determining region 2 (CDR2), and complementarity determining region 3 (CDR3) sequences of SEQ ID NO: 5;
and
b) the second antigen-binding region comprises a heavy chain variable region (VH) comprising the complementarity determining region 1 (CDR1), complementarity determining region 2 (CDR2), and complementarity determining region 3 (CDR3) sequences of SEQ ID NO: 8, and a light chain variable region (VL) comprising the complementarity determining region1 (CDR1), complementarity determining region 2 (CDR2), and complementarity determining region 3 (CDR3) sequences of SEQ ID NO: 12.

22. The method according to any one of the preceding claims, wherein
a) the first antigen-binding region comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, GAS, 7, respectively;
and
b) the second antigen-binding region comprises a heavy chain variable region (VH) comprising the CDR1, CDR2, and CDR3 sequences set forth in: SEQ ID NO: 9, 10, 11 respectively, and a light chain variable region (VL) comprising the CDR1, CDR2, and CDR3 sequences set forth in: SEQ ID NO: 13, DDN, 14, respectively.

23. The method according to any one of claims to 21 to 22, wherein each variable region comprises three complementarity determining regions (CDR1, CDR2, and CDR3) and four framework regions (FR1, FR2, FR3, and FR4).

24. The method according to claim 23, wherein said complementarity determining regions and said framework regions are arranged from amino-terminus to carboxy-terminus in the following order: FR1, CDR1, FR2, CDR2, FR3, CDR3, FR4.

25. The method according to any one of the preceding claims, wherein
a) The first antigen-binding region comprises a heavy chain variable region (VH) comprising an amino acid sequence having at least 90%, at least 95%, at least 97%, at least 99%, or 100% sequence identity to SEQ ID NO: 1 and a light chain variable region (VL) region comprising an amino acid sequence having at least 90%, at least 95%, at least 97%, at least 99%, or 100% sequence identity to SEQ ID NO: 5;
and
b) the second antigen-binding region comprises a heavy chain variable region (VH) comprising an amino acid sequence having at least 90%, at least 95%, at least 97%, at least 99%, or 100% sequence identity to SEQ ID NO: 8 and a light chain variable region (VL) region comprising an amino acid sequence having at least 90%, at least 95%, at least 97%, at least 99%, or 100% sequence identity to SEQ ID NO: 12.

26. The method according to any one of the preceding claims, wherein
a) The first antigen-binding region comprises a heavy chain variable region (VH) comprising the amino acid sequence set forth in SEQ ID NO: 1 and a light chain variable region (VL) region comprising the amino acid sequence set forth in SEQ ID NO: 5;
and
b) the second antigen-binding region comprises a heavy chain variable region (VH) comprising the amino acid sequence set forth in SEQ ID NO: 8 and a light chain variable region (VL) region comprising the amino acid sequence set forth in SEQ ID NO: 12.

27. The method according to any one of the preceding claims, wherein the binding agent is an antibody

28. The method according to any one of the preceding claims, wherein the binding agent is a multispecific antibody, such as a bispecific antibody.

29. The method according to any one of the preceding claims, wherein the binding agent has no more than two binding regions.

30. The method according to any one of the preceding claims, wherein the binding agent is in the format of a full-length antibody or an antibody fragment.

31. The method according to any one of the preceding claims, wherein the binding agent comprises
i) a polypeptide comprising, consisting of or consisting essentially of, said first heavy chain variable region (VH) and a first heavy chain constant region (CH), and
ii) a polypeptide comprising, consisting of or consisting essentially of, said second heavy chain variable region (VH) and a second heavy chain constant region (CH).

32. The method according to any one of the preceding claims, wherein the binding agent comprises
i) a polypeptide comprising said first light chain variable region (VL) and further comprising a first light chain constant region (CL), and
ii) a polypeptide comprising said second light chain variable region (VL) and further comprising a second light chain constant region (CL).

33. The method according to any one of the preceding claims, wherein the binding agent is an antibody comprising a first binding arm and a second binding arm, wherein the first binding arm comprises
i) a polypeptide comprising said first heavy chain variable region (VH) and said first heavy chain constant region (CH), and
ii) a polypeptide comprising said first light chain variable region (VL) and said first light chain constant region (CL);
and the second binding arm comprises
iii) a polypeptide comprising said second heavy chain variable region (VH) and said second heavy chain constant region (CH), and
iv) a polypeptide comprising said second light chain variable region (VL) and said second light chain constant region (CL).

34. The method according to any one of claims 31 to 33, wherein each of the first and second heavy chain constant regions (CH) comprises one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

35. The method according to any one of claims 31 to 34, wherein each of the first and second heavy chain constant regions (CHs) comprises a CH3 region and wherein the two CH3 regions comprise asymmetrical mutations.

36. The method according to any one of claims 31 to 35, wherein in said first heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.

37. The method according to claims 31 to 36, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

38. The method according to any of the preceding claims, wherein said binding agent induces Fc-mediated effector function to a lesser extent compared to another antibody comprising the same first and second antigen binding regions and two heavy chain constant regions (CHs) comprising human IgG1 hinge, CH2 and CH3 regions.

39. The method according to anyone of claims 31 to 38, wherein said first and second heavy chain constant regions (CHs) are modified so that the antibody induces Fc-mediated effector function to a lesser extent compared to an antibody which is identical except for comprising non-modified first and second heavy chain constant regions (CHs).

40. The method according to claim 39, wherein each of said non-modified first and second heavy chain constant regions (CHs) comprises the amino acid sequence set forth in SEQ ID NO: 15 or SEQ ID NO: 30.

41. The method according to any of claims 39 to 40, wherein said Fc-mediated effector function is measured by binding to Fcγ receptors, binding to C1q, or induction of Fc-mediated cross-linking of Fcγ receptors.

42. The method according to claim 41, wherein said Fc-mediated effector function is measured by binding to C1q.

43. The method according to any one of claims 31 to 42, wherein said first and second heavy chain constant regions have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

44. The method according to any one of the preceding claims, wherein in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

45. The method according to claim 44, wherein the positions corresponding to positions L234 and L235 in a human IgG1 heavy chain according to EU numbering are F and E, respectively, in said first and second heavy chains.

46. The method according to claim 44 or 45, wherein the positions corresponding to positions L234, L235, and D265 in a human IgG1 heavy chain according to EU numbering are F, E, and A, respectively, in said first and second heavy chain constant regions (HCs).

47. The method according to any one of claims 31 to 46, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

48. The method according to any one of claims 31 to 47, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

49. The method according to any one of claims 31 to 48, wherein the constant region of said first and/or second heavy chain comprises or consists essentially of or consists of an amino acid sequence selected from the group consisting of
a) the sequence set forth in SEQ ID NO: 15 or SEQ ID NO: 30 [IgG1-FC],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4, at the most 3, at the most 2 or at the most 1 substitution compared to the amino acid sequence defined in a) or b).

50. The method according to any one of claims 31 to 49, wherein the constant region of said first or second heavy chain, such as the first heavy chain, comprises or consists essentially of or consists of an amino acid sequence selected from the group consisting of
a) the sequence set forth in SEQ ID NO: 16 or SEQ ID NO: 31 [IgG1-F405L],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 9 substitutions, such as at the most 8, at the most 7, at the most 6, at the most 5, at the most 4, at the most 3, at the most 2 or at the most 1 substitution compared to the amino acid sequence defined in a) or b).

51. method according to any one of claims 31 to 50, wherein the constant region of said first or second heavy chain, such as the second heavy chain, comprises or consists essentially of or consists of an amino acid sequence selected from the group consisting of
a) the sequence set forth in SEQ ID NO: 17 or SEQ ID NO: 32 [IgG1-F409R]
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution compared to the amino acid sequence defined in a) or b).

52. The method according to any one of claims 31 to 51, wherein the constant region of said first and/or second heavy chain, comprises or consists essentially of, or consists of, an amino acid sequence selected from the group consisting of
a) the sequence set forth in SEQ ID NO: 18 or SEQ ID NO: 33 [IgG1-Fc_FEA],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 7 substitutions, such as at the most 6 substitutions, at the most 5, at the most 4, at the most 3, at the most 2 or at the most 1 substitution compared to the amino acid sequence defined in a) or b).

53. The method according to any one of claims 31 to 52, wherein the constant region of said first and/or second heavy chain, such as the first heavy chain, comprises or consists essentially of or consists of an amino acid sequence selected from the group consisting of
a) the sequence set forth in SEQ ID NO: 19 or SEQ ID NO: 34 [IgG1-Fc_FEAL],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4 substitutions, at the most 3, at the most 2 or at the most 1 substitution compared to the amino acid sequence defined in a) or b).

54. The method according to any one of claims 31 to 53, wherein the constant region of said first and/or second heavy chain, such as the second heavy chain, comprises or consists essentially of or consists of an amino acid sequence selected from the group consisting of
a) the sequence set forth in SEQ ID NO: 20 or SEQ ID NO: 35 [IgG1-Fc_FEAR]
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4, at the most 3, at the most 2 or at the most 1 substitution compared to the amino acid sequence defined in a) or b).

55. The method according to any one of the preceding claims, wherein said binding agent comprises a kappa (κ) light chain constant region.

56. The method according to any one of the preceding claims, wherein said binding agent comprises a lambda (λ) light chain constant region.

57. The method according to any one of the preceding claims, wherein said first light chain constant region is a kappa (κ) light chain constant region.

58. The method according to any one of the preceding claims, wherein said second light chain constant region is a lambda (λ) light chain constant region.

59. The method according to any one of the preceding claims, wherein said first light chain constant region is a lambda (λ) light chain constant region.

60. The method according to any one of the preceding claims, wherein said second light chain constant region is a kappa (κ) light chain constant region.

61. The method according to any one of claims 55 to 60, wherein the kappa (κ) light chain comprises an amino acid sequence selected from the group consisting of
a) the sequence set forth in SEQ ID NO: 21,
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

62. The method according to any one of claims 56 to 61, wherein the lambda (λ) light chain comprises an amino acid sequence selected from the group consisting of
a) the sequence set forth in SEQ ID NO: 22,
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

63. The method according to any one of the preceding claims, wherein the binding agent is of an isotype selected from the group consisting of IgG1, IgG2, IgG3, and IgG4.

64. The method according to any one of the preceding claims, wherein the binding agent is a full-length IgG1 antibody.

65. The method according to any one of the preceding claims, wherein said antibody is of the IgG1m(f) allotype.

66. The method according to any of the preceding claims, wherein the binding agent comprises
(i) a first binding arm comprising a first heavy chain variable region (VH) and a first light chain variable region (VL),
wherein the first VH comprises a first HCDR1 sequence, a first HCDR2 sequence, and a first HCDR3 sequence, wherein the first HCDR1 sequence is set forth in SEQ ID NO: 2, wherein the first HCDR2 sequence is set forth in SEQ ID NO: 3, and wherein the first HCDR3 sequence comprises is set forth in SEQ ID NO: 4; and wherein the first VL comprises a first LCDR1 sequence, a first LCDR2 sequence, and a first LCDR3 sequence, wherein the first LCDR1 sequence is set forth in SEQ ID NO: 6, wherein the first LCDR2 sequence is GAS, and wherein the first LCDR3 sequence is set forth in SEQ ID NO: 7; and
(ii) a second binding arm comprising a second heavy chain variable region (VH) and a second light chain variable region (VL),
wherein the second VH comprises a second HCDR1 sequence, a second HCDR2 sequence, and a second HCDR3 sequence, wherein the second HCDR1 sequence is set forth in SEQ ID NO: 9, wherein the second HCDR2 sequence is set forth in SEQ ID NO: 10, and wherein the second HCDR3 sequence is set forth in SEQ ID NO: 11; and wherein the second VL comprises a second LCDR1 sequence, a second LCDR2 sequence, and a second LCDR3 sequence, wherein the second LCDR1 sequence is set forth in SEQ ID NO: 13, wherein the second LCDR2 sequence is DDN, and wherein the second LCDR3 sequence is set forth in SEQ ID NO: 14;
wherein the first binding arm comprises a first heavy chain constant region (CH) and the second binding arm comprises a second CH, wherein positions L234, L235, and D265 in a human IgGI heavy chain according to EU numbering are F, E, and A, respectively, in the first CH and the second CH; and
wherein the amino acid in the position corresponding to F405 in a human IgGI heavy chain according to EU numbering is L in the first CH and the amino acid in the position corresponding to K409 in a human IgGI heavy chain according to EU numbering is R in the second CH.

67. The method according to any of the preceding claims, wherein the binding agent comprises
(i) a first binding arm comprising a first heavy chain variable region (VH) and a first light chain variable region (VL),
wherein the first VH comprises a first HCDR1 sequence, a first HCDR2 sequence, and a first HCDR3 sequence, wherein the first HCDR1 sequence is set forth in SEQ ID NO: 2, wherein the first HCDR2 sequence is set forth in SEQ ID NO: 3, and wherein the first HCDR3 sequence is set forth in SEQ ID NO: 4, and wherein the first VL comprises a first LCDR1 sequence, a first LCDR2 sequence, and a first LCDR3 sequence, wherein the first LCDR1 sequence is set forth in SEQ ID NO: 6, wherein the first LCDR2 sequence is GAS, and wherein the first LCDR3 sequence is set forth in SEQ ID NO: 7; and
(ii) a second binding arm comprising a second heavy chain variable region (VH) and a second light chain variable region (VL), wherein the second VH comprises a second HCDR1 sequence, a second HCDR2 sequence, and a second HCDR3 sequence, wherein the second HCDR1 sequence is set forth in SEQ ID NO: 9, wherein the second HCDR2 sequence is set forth in SEQ ID NO: 10, and wherein the second HCDR3 sequence is set forth in SEQ ID NO: 11, and wherein the second VL comprises a second LCDR1 sequence, a second LCDR2 sequence, and a second LCDR3 sequence, wherein the second LCDR1 sequence is set forth in SEQ ID NO: 13, wherein the second LCDR2 sequence is DDN, and wherein the second LCDR3 sequence is set forth in SEQ ID NO: 14;
wherein the first binding arm comprises a first heavy chain constant region (CH) and the second binding arm comprises a second CH, wherein positions L234, L235, and D265 in a human IgGI heavy chain according to EU numbering are F, E, and A, respectively, in the first CH and the second CH; and
wherein the amino acid in the position corresponding to K409 in a human IgGI heavy chain according to EU numbering is R in the first CH and the amino acid in the position corresponding to F405 in a human IgGI heavy chain according to EU numbering is L in the second CH.

68. The method according to any of the preceding claims, wherein the binding agent comprises
(i) a first binding arm comprising a first heavy chain variable region (VH) and a first light chain variable region (VL),
wherein the first VH comprises a first HCDR1 sequence, a first HCDR2 sequence, and a first HCDR3 sequence, wherein the first HCDR1 sequence comprises an amino acid sequence as set forth in SEQ ID NO: 9, wherein the first HCDR2 sequence comprises an amino acid sequence as set forth in SEQ ID NO: 10, and wherein the first HCDR3 sequence comprises an amino acid sequence as set forth in SEQ ID NO: 11, and wherein the first VL comprises a first LCDR1 sequence, a first LCDR2 sequence, and a first LCDR3 sequence, wherein the first LCDR1 sequence comprises an amino acid sequence as set forth in SEQ ID NO: 13, wherein the first LCDR2 sequence comprises the amino acid sequence GAS, and wherein the first LCDR3 sequence comprises an amino acid sequence as set forth in SEQ ID NO: 14; and
(ii) a second binding arm comprising a second heavy chain variable region (VH) and a second light chain variable region (VL),
wherein the second VH comprises a second HCDR1 sequence, a second HCDR2 sequence, and a second HCDR3 sequence, wherein the second HCDR1 sequence comprises an amino acid sequence as set forth in SEQ ID NO: 18, wherein the second HCDR2 sequence comprises an amino acid sequence as set forth in SEQ ID NO: 19, and wherein the second HCDR3 sequence comprises an amino acid sequence as set forth in SEQ ID NO: 20, and wherein the second VL comprises a second LCDR1 sequence, a second LCDR2 sequence, and a second LCDR3 sequence, wherein the second LCDR1 sequence comprises an amino acid sequence as set forth in SEQ ID NO: 22, wherein the second LCDR2 sequence comprises the amino acid sequence DDN, and wherein the second LCDR3 sequence comprises an amino acid sequence as set forth in SEQ ID NO: 23;
wherein the first binding arm comprises a first heavy chain constant region (CH) and the second binding arm comprises a second CH, wherein positions L234, L235, and D265 in a human IgGI heavy chain according to EU numbering are F, E, and A, respectively, in the first CH and the second CH; and
wherein the amino acid in the position corresponding to F405 in a human IgGI heavy chain according to EU numbering is L in the first CH and the amino acid in the position corresponding to K409 in a human IgGI heavy chain according to EU numbering is R in the second CH.

69. The method according to any of the preceding claims, wherein the binding agent comprises
(i) a first binding arm comprising a first heavy chain variable region (VH) comprising the amino acid sequence as set forth in SEQ ID NO: 1, and a first light chain variable region (VL) comprising the amino acid sequence as set forth in SEQ ID NO: 5; and
(ii) a second binding arm comprising a second heavy chain variable region (VH) comprising the amino acid sequence as set forth in SEQ ID NO: 8, and second light chain variable region (VL) comprising the amino acid sequence as set forth in SEQ ID NO: 12;
wherein the first binding region comprises a first heavy chain constant region (CH) and the second binding arm comprises a second CH, wherein positions L234, L235, and D265 in a human IgGI heavy chain according to EU numbering are F, E, and A, respectively, in the first CH and the second CH; and
wherein the amino acid in the position corresponding to K409 in a human IgGI heavy chain according to EU numbering is R in the first CH and the amino acid in the position corresponding to F405 in a human IgGI heavy chain according to EU numbering is L in the second CH.

70. The method according to any of the preceding claims, wherein the binding agent comprises
(i) a first binding arm comprising a first heavy chain variable region (VH) comprising the amino acid sequence as set forth in SEQ ID NO: 1, and a first light chain variable region (VL) comprising the amino acid sequence as set forth in SEQ ID NO: 5; and
(ii) a second binding arm comprising a second heavy chain variable region (VH) comprising the amino acid sequence as set forth in SEQ ID NO: 8, and second light chain variable region (VL) comprising the amino acid sequence as set forth in SEQ ID NO: 12;
wherein the first binding arm comprises a first heavy chain constant region (CH) and the second antigen binding region comprises a second CH, wherein positions L234, L235, and D265 in a human IgGI heavy chain according to EU numbering are F, E, and A, respectively, in the first CH and the second CH; and
wherein the amino acid in the position corresponding to F405 in a human IgGI heavy chain according to EU numbering is L in the first CH and the amino acid in the position corresponding to K409 in a human IgGI heavy chain according to EU numbering is R in the second CH.

71. The method according to any of the preceding claims, wherein the binding agent comprises
i) A first heavy chain comprising, consisting essentially of or consisting of the amino acid sequence as set forth in SEQ ID NO: 36, and a first light chain comprising, consisting essentially of or consisting of the amino acid sequence as set forth in SEQ ID NO: 37; and
ii) a second heavy chain comprising, consisting essentially of or consisting of the amino acid sequence as set forth in SEQ ID NO: 38, and a second light chain comprising, consisting essentially of or consisting of the amino acid sequence as set forth in SEQ ID NO: 39.

72. The binding agent according to any one of the preceding claims, wherein the binding agent is in the format of a full-length antibody or an antibody fragment.

73. The method according to any one of the preceding claims wherein the binding agent is acasunlimab or a biosimilar thereof.

74. The method according to any one of the preceding claims wherein the binding agent is in a composition or formulation comprising histidine, sucrose and Polysorbate-80, and has a pH from 5 to 6.

75. The method according to any one of the preceding claims wherein the binding agent is in a composition or formulation comprising about 20 mM histidine, about 250 mM Sucrose, about 0.02% Polysorbate-80, and having a pH of about 5.5.

76. The method according to any one of the preceding claims wherein the binding agent is in a composition or formulation comprising 10-30 mg binding agent/mL, such as 20 mg binding agent/mL.

77. The method according to any one of the preceding claims, wherein the binding agent is in a composition as defined in any one of claims 75 and 75, and is diluted in 0.9% NaCl (saline) prior to administration.

78. method according to any one of the preceding claims, wherein the subject is a human subject.

79. The method according to any one of the preceding claims, wherein the tumor or cancer is a solid tumor.

80. The method according to any one of the preceding claims, wherein said tumor is a PD-L1 positive tumor.

81. The method according to any one of the preceding claims, wherein the tumor or cancer is selected from the group consisting of melanoma, ovarian cancer, lung cancer (e.g. non-small cell lung cancer (NSCLC), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, prostate cancer, penile cancer, cervical cancer, Hodgkin's lymphoma, non-Hodgkin's lymphoma, Merkel cell carcinoma and mesothelioma.

82. The method according to any one of the preceding claims, wherein the tumor or cancer is selected from the group consisting of lung cancer (e.g. non-small cell lung cancer (NSCLC), urothelial cancer (cancer of the bladder, ureter, urethra, or renal pelvis), endometrial cancer (EC), breast cancer (e.g. triple negative breast cancer (TNBC)), squamous cell carcinoma of the head and neck (SCCHN) (e.g. cancer of the oral cavity, pharynx or larynx) and cervical cancer.

83. The method according to any one of the preceding claims, wherein the tumor or cancer is a lung cancer.

84. The method according to claim 83, wherein the lung cancer is a non-small cell lung cancer (NSCLC), such as a squamous or non-squamous NSCLC.

85. The method according to claim 84, wherein the NSCLC does not have an epidermal growth factor (EGFR)-sensitizing mutation and/or anaplastic lymphoma (ALK) translocation/ROS1 rearrangement.

86. The method according to any one of the preceding claims, wherein the subject has received one, two, three or four prior systemic treatment regimens, such as for advanced/metastatic disease, and has experienced disease progression on or after last prior systemic treatment, such as disease progression determined by radiography.

87. The method according to claim 86, wherein the subject has received platinum-based chemotherapy.

88. The method according to any one of claims 1 to 86, wherein the subject is not eligible for platinum-based therapy and has alternative chemotherapy, e.g. a treatment with gemcitabine-containing regimen.

89. The method according to any one of the preceding claims, wherein the subject has received prior treatment with checkpoint inhibitor(s), such as agent(s) targeting PD-1/PD-L, such as a PD-1/PD-L1 inhibitor.

90. The method according to any one of the preceding claims, wherein the subject has experienced disease progression on or after treatment with checkpoint inhibitor(s), such as agent(s) targeting PD-1/PD-L, such as a PD-1/PD-L1 inhibitor.

91. The method according to any one of the preceding claims, wherein the subject has experienced disease progression on or after last prior treatment with checkpoint inhibitor(s), such as agent(s) targeting PD-1/PD-L, such as a PD-1/PD-L1 inhibitor.

92. The method according to any one of the preceding claims, wherein the subject has experienced disease progression on or after last prior systemic treatment, such as disease progression determined by radiography.

93. The method according to any one of the preceding claims, wherein last prior treatment of the subject was with a PD1 inhibitor or PD-L1 inhibitor, such as an anti PD-1 antibody or an anti-PD-L1 antibody, the PD-1 inhibitor or PD-L1 inhibitor being administered as monotherapy or as part of a combination therapy.

94. The method according to any one of the preceding claims, wherein the time from progression on last treatment with a PD1 inhibitor or PD-L1 inhibitor, such as an anti PD-1 antibody or an anti-PD-L1 antibody is 8 months or less, such as 7 months or less, 6 months or less, 5 months or less, 4 months or less, 3 months or less, 2 months or less, 1 month or less, 3 weeks or less or such as 2 weeks or less.

95. The method according to any one of the preceding claims, wherein the time from last dosing of a PD1 inhibitor or PD-L1 inhibitor, such as an anti PD-1 antibody or an anti-PD-L1 antibody as part of last prior treatment is 8 months or less, such as 7 months or less, 6 months or less, 5 months or less, 4 months or less, 3 months or less, 2 months or less, 1 month or less, 3 weeks or less or such as 2 weeks or less.

96. The method according to any one of claims 1-92, wherein the subject has not received prior treatment with checkpoint inhibitor(s), such as agent(s) targeting PD-1/PD-L, such as a PD-1/PD-L1 inhibitor.

97. The method according to any one of claims 1-92, the method being for first line treatment of said tumor or cancer.

98. The method according to any one of the preceding claims, the method being for second line treatment of said tumor or cancer.

99. A binding agent for use in reducing or preventing progression of a tumor or for use in treatment of cancer, wherein the binding agent comprises a first antigen-binding region binding to human CD137, such as human CD137 consisting of the amino acid sequence set forth in SEQ ID NO: 24, and a second antigen-binding region binding to human PD-L1, such as human PD-L1 consisting of the amino acid sequence set forth in SEQ ID NO: 26, and the binding agent is administered to the subject in a dosing schedule that comprises administration of Dose A in one or more treatment cycles and administration of dose B in one or more treatment cycles,
the amount of binding agent in Dose A being
a) about 0.3-2.5 mg/kg body weight, or about 25-200 mg in total; and/or
b) about 2.1×10⁻⁹-1.7×10⁻⁸ mol/kg body weight, or about 1.7×10⁻⁷-1.4×10⁻⁶ mol in total; and
the amount of binding agent in Dose B being
c) about 3.8-7.5 mg/kg body weight, or about 300-600 mg in total; and/or
d) about 2.6×10⁻⁸-5.1×10⁻⁸ mol/kg body weight, or about 2.0-4.1×10⁻⁶ mol in total.

100. The binding agent for use according to claim 96, wherein the dosing schedule is as defined in any one of claims 1 to 20.

101. The binding agent for use according to claim 96 or 97, wherein the binding agent is as defined in any one of claims 21 to 73.


SEQUENCE LISTING (39 sequences)

SEQ ID NO: 1 - protein, 117 aa, Artificial sequence
  Antibody variable region
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSS

SEQ ID NO: 2 - protein, 8 aa, Artificial sequence
  CDR sequence
GFSLNDYW

SEQ ID NO: 3 - protein, 7 aa, Artificial sequence
  CDR sequence
IDVGGSL

SEQ ID NO: 4 - protein, 11 aa, Artificial sequence
  CDR sequence
ARGGLTYGFDL

SEQ ID NO: 5 - protein, 110 aa, Artificial sequence
  Antibody variable region
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIK

SEQ ID NO: 6 - protein, 6 aa, Artificial sequence
  CDR sequence
EDISSY

SEQ ID NO: 7 - protein, 12 aa, Artificial sequence
  CDR sequence
HYYATISGLGVA

SEQ ID NO: 8 - protein, 121 aa, Artificial sequence
  Antibody variable region
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSS

SEQ ID NO: 9 - protein, 8 aa, Artificial sequence
  CDR sequence
GSTFSTYA

SEQ ID NO: 10 - protein, 8 aa, Artificial sequence
  CDR sequence
FSGSGGFT

SEQ ID NO: 11 - protein, 14 aa, Artificial sequence
  CDR sequence
AIPARGYNYGSFQH

SEQ ID NO: 12 - protein, 108 aa, Artificial sequence
  Antibody variable region
SYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVL

SEQ ID NO: 13 - protein, 6 aa, Artificial sequence
  CDR sequence
NIGSKS

SEQ ID NO: 14 - protein, 11 aa, Artificial sequence
  CDR sequence
QVWDSSSDHVV

SEQ ID NO: 15 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 330 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 17 - protein, 330 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 18 - protein, 330 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 330 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 330 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 21 - protein, 107 aa, Homo sapiens [sequence omitted]

SEQ ID NO: 22 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 254 aa, Homo sapiens
MGNSCYNIVALLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 24 - protein, 232 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 25 - protein, 289 aa, Homo sapiens
MRIFAVFIFMTYWHLLNAFTVTVPKDLYVVEYGSNMTIECFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 26 - protein, 272 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 27 - protein, 1209 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 28 - protein, 114 aa, Artificial sequence
  Antibody variable region
QSLEESGGRLVTPGTPLTLTCTVSGFSLNDYWMSWVRQAPGKGLEWIGYIDVGGSLYYASWAKGRFTISRTSTTVDLKMTSLTTEDTATYFCARGGLTYGFDLWGPGTLVTVSS

SEQ ID NO: 29 - protein, 110 aa, Artificial sequence
  Antibody variable region
DIVMTQTPASVSEPVGGTVTINCQASEDISSYLAWYQQKPGQRPKRLIYGASDLASGVPSRFSASGSGTEYALTISDLESADAATYYCHYYATISGLGVAFGGGTEVVVK

SEQ ID NO: 30 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 329 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 32 - protein, 329 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 33 - protein, 329 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 329 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 35 - protein, 329 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 36 - protein, 446 aa, Artificial sequence
  Antibody heavy chain
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 37 - protein, 217 aa, Artificial sequence
  Antibody light chain
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 450 aa, Artificial sequence
  Antibody heavy chain
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 39 - protein, 214 aa, Artificial sequence
  Antibody light chain
SYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS